(19) United States
(12) Patent Application Publication
(10) Pub. No.: US 2024/0043534 A1
(43) Pub. Date: Feb. 8, 2024

(54) METHODS AND COMPOSITIONS COMPRISING MODIFIED FAB SCAFFOLDS AND PROTEIN G FAB BINDING DOMAINS

(71) Applicant: The University of Chicago, Chicago, IL (US)

(72) Inventors: Anthony KOSSIAKOFF, Chicago, IL (US); Tomasz SLEZAK, Chicago, IL (US); Elena DAVYDOVA, Chicago, IL (US); Edwin ARAUZ-DIAZ, Chicago, IL (US)

(73) Assignee: The University of Chicago, Chicago, IL (US)

(21) Appl. No.: 17/754,797

(22) PCT Filed: Oct. 14, 2020
(86) PCT No.: PCT/US2020/070664

§ 371 Date: Apr. 12, 2022

Related U.S. Application Data
(60) Provisional application No. 62/914,851, filed on Oct. 14, 2019.

(51) Int. Cl. C07K 16/28; A61P 35/00

(52) U.S. Cl. CPC C07K 16/2809; C07K 16/2887; A61P 35/00; C07K 16/2803; C07K 2317/52; C07K 2317/55; C07K 2319/03

(57) ABSTRACT

The engineered polypeptide comprising modified Fab constant regions and/or protein G Fab binding domains provide advanced affinity reagents that can be used in cell biology applications as well as for therapeutic applications. Accordingly, aspects of the disclosure relate to a polypeptide comprising a constant region of an antibody light chain, wherein the constant region comprises a substitution/deletion of amino acids corresponding to positions 16-20 of SEQ ID NO:1 of the constant region with the amino acids LRT.

### CROSS-REFERENCE TO RELATED APPLICATIONS

This application claims the benefit of priority of U.S. Provisional Patent Application No. 62/914,851 filed Oct. 14, 2019, which is hereby incorporated by reference in its entirety.

The invention was made with government support under Grant No. GM117372 awarded by the National Institutes of Health. The government has certain rights in the invention.

### BACKGROUND OF THE DISCLOSURE

### 1. Field of the Disclosure

This disclosure relates to modified Fab-binding regions from protein G that are useful as therapeutics, in protein purification, in diagnostic assays, and in biochemical and immunological assays.

### 2. Description of Related Art

Immunoglobulin binding proteins (IBPs) are broadly used as reagents for the purification and detection of antibodies. Among the IBPs, the most widely used are Protein-A and Protein-G. The C2 domain of Protein-G from Streptococcus is a multi-specific protein domain (Bjorck and Kronvall, 1984); it possesses a high affinity (KD ˜10 nM) for the Fc region of the IgG, but a much lower affinity (KD ˜low μM) for the constant domain of the antibody fragment (Fab), which limits some of its applications. Therefore, there is a need in the art for IBPs that have a higher affinity for the Fab domain.

### SUMMARY OF THE DISCLOSURE

The engineered polypeptide comprising modified Fab constant regions and/or protein G Fab binding domains fulfill a need in the art by providing advanced affinity reagents that can be used in cell biology applications as well as for therapeutic applications. Accordingly, aspects of the disclosure relate to a polypeptide comprising a constant region of an antibody light chain, wherein the constant region comprises a substitution/deletion of amino acids corresponding to positions 16-20 of SEQ ID NO:1 of the constant region with the amino acids LRT. SEQ ID NO:1 corresponds to a kappa light chain constant region: RTVAAPSVFIFPPSDEQLKSGTASVVCLLNNFYPREAKVQWKVDNALQSGNSQESVT EQDSKDSTYSLSSTLTLSKADYEKHKVYACEVTHQGLSSPVTKSFNRGEC (SEQ ID NO:1). Further aspects relate to a polypeptide comprising a constant region of an antibody light chain, wherein the constant region comprises a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

Yet further aspects relate to a polypeptide comprising a Fab comprising a heavy chain region and a light chain region, wherein the light chain region comprises a constant region comprising a substitution/deletion of amino acids corresponding to positions 16-20 of SEQ ID NO:1 of the constant region with the amino acids LRT wherein the Fab is conjugated to a protein G Fab binding domain comprising a modified isotype recognition region, wherein the isotype recognition region is modified to YAYVHE (SEQ ID NO:9), YAFGNG (SEQ ID NO:10), or IDMVSS (SEQ ID NO:11). In some aspects, the polypeptide comprising a Fab comprises a a heavy chain region and a light chain region, wherein the light chain region comprises a constant region comprising a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T, wherein the Fab is conjugated to a protein G Fab binding domain comprising a modified isotype recognition region, wherein the isotype recognition region is modified to YAYVHE (SEQ ID NO:9), YAFGNG (SEQ ID NO:10), or IDMVSS (SEQ ID NO:11).

Also described herein is a polypeptide comprising a Fab conjugated to a protein G Fab binding domain comprising a modified isotype recognition region, wherein the isotype recognition region is modified to YAYVHE (SEQ ID NO:9), YAFGNG (SEQ ID NO:10), or IDMVSS (SEQ ID NO:11).

In some aspects, the disclosure relates to a polypeptide comprising a Fab comprising a heavy chain region and a light chain region, wherein the light chain region comprises a constant region comprising a substitution/deletion of amino acids corresponding to positions 16-20 of SEQ ID NO:1 of the constant region with the amino acids LRT and wherein the heavy and/or light chain region of the Fab is conjugated through a linker to a polypeptide comprising a peptide spacer, a transmembrane domain, and an endodomain. Related aspects comprise a polypeptide comprising a Fab comprising a heavy chain region and a light chain region, wherein the light chain region comprises a constant region comprising a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T, and wherein the heavy and/or light chain region of the Fab is conjugated through a linker to a polypeptide comprising a peptide spacer, a transmembrane domain, and an endodomain.

The term “corresponding to” is intended to mean the amino acid position that aligns with the amino acid position at said position of SEQ ID NO:1. For example, amino acids 16-23 of SEQ ID NOS:12-16 correspond to amino acids 15-22 of SEQ ID NO:1, as shown by the sequence alignment of FIG. 21.

Further aspects relate to a polypeptide comprising a protein G Fab binding domain comprising a modified isotype recognition region, wherein the isotype recognition region is modified to YAYVHE (SEQ ID NO:9), YAFGNG (SEQ ID NO:10), or IDMVSS (SEQ ID NO:11), and wherein the polypeptide further comprises a peptide spacer, a transmembrane domain, and an endodomain.

Further aspects relate to a Fab comprising a constant region of an antibody light chain, wherein the constant region comprises a substitution/deletion of amino acids corresponding to positions 16-20 of SEQ ID NO:1 of the constant region with the amino acids LRT. Yet further aspects relate to a Fab comprising a constant region of an antibody light chain, wherein the constant region comprises a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

The disclosure also describes a polypeptide comprising a modified protein G Fab binding domain comprising an isotype recognition region having the following amino acid sequence: YAFGNG (SEQ ID NO:10). Exemplary embodiments include wherein the polypeptide comprises an amino acid sequence with at least 70% sequence identity to SEQ ID NO:4 or 7. In some embodiments, the polypeptide comprises an amino acid sequence with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity (or any derivable range therein) to SEQ ID NO:4 or 7. In another aspect, the disclosure provides for a polypeptide comprising a modified protein G Fab binding domain comprising an isotype recognition region having the following amino acid sequence: IDMVSS (SEQ ID NO:11). Exemplary embodiments, include wherein the polypeptide comprises an amino acid sequence with at least 70% sequence identity to SEQ ID NO:5 or 8. In some embodiments, the polypeptide comprises an amino acid sequence with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity (or any derivable range therein) to SEQ ID NO:5 or 8.

Yet further aspects relate to a polypeptide comprising a modified protein G Fab binding domain comprising an isotype recognition region having the following amino acid sequence: YAYVHE (SEQ ID NO:9) and wherein the protein G Fab binding domain further comprises a substitution of the amino acid corresponding to position 19 of SEQ ID NO:23.

Further aspects relate to a polypeptide comprising a Fab conjugated to a protein G Fab binding domain comprising a modified isotype recognition region, wherein the isotype recognition region is modified to YAYVHE (SEQ ID NO:9) and wherein the Fab specifically binds to a T cell surface receptor. Further aspects relate to a polypeptide comprising a Fab comprising a heavy chain region and a light chain region, wherein the light chain region comprises a kappa constant region comprising a substitution/deletion of amino acids corresponding to positions 16-20 of SEQ ID NO:1 of the constant region with the amino acids LRT and wherein the heavy and/or light chain region of the Fab is conjugated through a linker to a polypeptide comprising a peptide spacer, a transmembrane domain, and an endodomain. Yet further aspects relate to a polypeptide comprising a protein G Fab binding domain comprising a modified isotype recognition region, wherein the isotype recognition region is modified to YAYVHE (SEQ ID NO:9), and wherein the polypeptide further comprises a peptide spacer, a transmembrane domain, and an endodomain. The disclosure also relates to a polypeptide comprising a Fab comprising a heavy chain region and a kappa light chain region, wherein the light chain region comprises a constant region comprising a substitution/deletion of amino acids corresponding to positions 16-20 of SEQ ID NO:1 of the constant region with the amino acids LRT wherein the Fab is conjugated to a protein G Fab binding domain comprising a modified isotype recognition region and wherein the isotype recognition region is modified to YAYVHE (SEQ ID NO:9); and further wherein the Fab specifically binds to a T cell surface receptor.

Also provided by the disclosure are nucleic acid encoding for the polypeptide of the disclosure or encoding the heavy or light chain of a Fab of the disclosure. Host cells comprising the polypeptides, Fabs, or nucleic acids of the disclosure are also contemplated. The disclosure also relates to therapeutic cells comprising nucleic acids encoding the polypeptides of the disclosure and/or polypeptides of the disclosure. The disclosure also relates to pharmaceutical compositions comprising the polypeptides, Fabs, nucleic acids, or therapeutic cells of the disclosure.

Method aspects of the disclosure relate to a method comprising expressing a nucleic of the disclosure in a host cell and isolating the polypeptides expressed from the nucleic acid. Further method aspects relate to a method for treating a subject comprising administering a polypeptide, Fab, or therapeutic cell of the disclosure.

Further aspects relate to a method for treating cancer in a subject comprising administering: a) a polypeptide comprising a first Fab conjugated to a protein G Fab binding domain comprising a modified isotype recognition region, wherein the isotype recognition region is modified to YAYVHE (SEQ ID NO:9) and wherein the Fab specifically binds to a T cell surface receptor; and b) a polypeptide comprising a second Fab that specifically binds to a tumor antigen; and wherein the second Fab comprises a kappa constant region of an antibody light chain, wherein the constant region comprises: i) a substitution/deletion of amino acids corresponding to positions 16-20 of SEQ ID NO:1 of the constant region with the amino acids LRT; or ii) a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

Further method aspects relate to a method for treating cancer in a subject comprising administering a T cell comprising: a) a polypeptide comprising a Fab comprising a heavy chain region and a light chain region, wherein the light chain region comprises a kappa constant region comprising a substitution/deletion of amino acids corresponding to positions 16-20 of SEQ ID NO:1 of the constant region with the amino acids LRT and wherein the heavy and/or light chain region of the Fab is conjugated through a linker to a polypeptide comprising a peptide spacer, a transmembrane domain, and an endodomain; and wherein the Fab specifically binds to a tumor antigen; or b) a nucleic acid encoding a polypeptide comprising a Fab comprising a heavy chain region and a light chain region, wherein the light chain region comprises a kappa constant region comprising a substitution/deletion of amino acids corresponding to positions 16-20 of SEQ ID NO:1 of the constant region with the amino acids LRT and wherein the heavy and/or light chain region of the Fab is conjugated through a linker to a polypeptide comprising a peptide spacer, a transmembrane domain, and an endodomain; and wherein the Fab specifically binds to a tumor antigen.

Further aspects relate to a method for treating cancer in a subject comprising administering: a) a T cell comprising: i) a polypeptide comprising a protein G Fab binding domain comprising a modified isotype recognition region, wherein the isotype recognition region is modified to YAYVHE (SEQ ID NO:9), and wherein the polypeptide further comprises a peptide spacer, a transmembrane domain, and an endodomain; or ii) a nucleic acid encoding a polypeptide comprising a protein G Fab binding domain comprising a modified isotype recognition region, wherein the isotype recognition region is modified to YAYVHE (SEQ ID NO:9), and wherein the polypeptide further comprises a peptide spacer, a transmembrane domain, and an endodomain; and b) a polypeptide comprising a Fab that specifically binds to a tumor antigen; and wherein the Fab comprises a kappa constant region of an antibody light chain, wherein the constant region comprises: i) a substitution/deletion of amino acids corresponding to positions 16-20 of SEQ ID NO:1 of the constant region with the amino acids LRT; or ii) a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

Further aspects relate to a method for detecting an antigen in a sample comprising a) incubating the sample with: i) a first polypeptide comprising at least one protein G Fab binding domain operatively linked to a first component of a detection pair; ii) a second polypeptide comprising at least one protein G Fab-binding domain operatively linked to a second component of a detection pair; iii) a first Fab optionally linked or bound to the first modified protein G Fab-binding domain that specifically binds to a first epitope on the antigen; and iv) a second Fab optionally linked or bound to the second modified protein G Fab-binding domain that specifically binds to a first epitope on the antigen; and b) detecting the detection pair.

Yet further aspects relate to a kit comprising a) a first polypeptide comprising a protein G Fab-binding domain operatively linked to a first component of a detection pair; and b) a second polypeptide comprising a protein G Fab-binding domain operatively linked to a second component of a detection pair.

In some embodiments, the constant region comprises the amino acid sequence of DLRTGT (SEQ ID NO: 17) in substitution for the amino acids corresponding to positions 15-22 of SEQ ID NO:1. In some embodiments, the polypeptide comprises a light chain constant region of SEQ ID NO:2 or a light chain constant region having at least 70% sequence identity to SEQ ID NO:2. In some embodiments, the polypeptide comprises a light chain constant region having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity to SEQ ID NO:2.

In some embodiments, the antibody light chain comprises a kappa antibody light chain. In some embodiments, the constant region comprises the amino acid sequence of DLRTGT (SEQ ID NO:17) in substitution for the amino acids at positions corresponding to 16-23 of SEQ ID NOS:12-16 of a lambda antibody light chain. In some embodiments, the polypeptide comprises an antibody light chain comprising a variable region and a constant region. In some embodiments, the polypeptide further comprises an antibody heavy chain, or a fragment thereof. The heavy chain, or fragment thereof may further comprise a heavy chain variable region and a heavy chain constant region. The polypeptide may comprise a fragment of a heavy chain, such as a heavy chain region of a fragment antigen binding (Fab). The heavy chain or heavy chain fragment may be carboxy-proximal to the light chain constant region. In alternative embodiments, the antibody heavy chain or fragment thereof is amino-proximal to the light chain constant region. A first region is carboxy-proximal to a second region when the first region is attached to the carboxy terminus of the second region. There may be further intervening amino acid residues between the first and second regions. Thus, the regions need not be immediately adjacent, unless specifically specified as not having intervening amino acid residues. The term “amino-proximal” is similarly defined in that a first region is amino-proximal to a second region when the first region is attached to the amino terminus of the second region. Similarly, there may be further intervening amino acid residues between the first and second regions unless stated otherwise.

In some embodiments, the polypeptide comprises an antigen binding fragment or a further antigen binding fragment. The antigen binding fragment may be one described herein. For example, the antigen binding fragment may comprise one or more of a single chain variable fragment (scFv), a single domain antibody, a single chain antibody, and the heavy and/or light chain of a Fab. These and other antigen binding fragments are further described throughout the disclosure. The antigen binding fragment may also be a Fab, such as a Fab comprising a modified light chain constant region described herein or an unmodified Fab. In some embodiments, the heavy and or light chain of the polypeptide and/or the antigen binding fragment specifically binds to a tumor antigen, an inflammatory or anti-inflammatory cytokine, a T cell surface receptor, a microbial antigen, a bacterial antigen, or a cell-specific surface protein.

In some embodiments, the polypeptide comprises a heavy and light chain comprising variable regions that specifically bind to a T cell surface receptor, and wherein the T cell surface receptor comprises CD3. The term “specifically bind” is used to indicate a specific association from, such as an association of an antibody and it's antigen. The K_{D} may be at least or at most about 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶ or any derivable range therein. In some embodiments, the antigen binding fragment is carboxy-proximal to the light chain constant region. In alternative embodiments, the antigen binding fragment is amino-proximal to the light chain constant region.

In some embodiments, the polypeptide further comprises a Fab binding domain. In some embodiments, the Fab binding domain comprises a protein G Fab binding domain. The protein G Fab binding domain may be a modified protein G Fab binding domain, such as one of the modified protein G Fab binding domains described herein. These include the protein G Fab binding domains comprising modified isotype regions, such as SEQ ID NOS:9-11 and 48-55. In some embodiments, the modified protein G Fab binding domain comprising a modified isotype recognition region, wherein the isotype recognition region is modified to YAYVHE (SEQ ID NO:9), YAFGNG (SEQ ID NO:10), or IDMVSS (SEQ ID NO:11). In some embodiments, the protein G Fab binding domain comprises one of SEQ ID NO:3-5 or 256. These exemplary protein G Fab binding domains include:

(SEQ ID NO: 3)
RTLSGYTTTTAVDAATAEKVFKQYAYVHE,
(SEQ ID NO: 4)
RTLSGYTTTTAVDAATAEKVFKQYAFGNG,
(SEQ ID NO: 5)
RTLSGYTTTTAVDAATAEKVFKQIDMVSS;
and
(SEQ ID NO: 256)
RTLSGYTTTTAVDAATAEEVFKQYAYVHE.

In some embodiments, the polypeptide comprises an amino acid sequence with at least 70% sequence identity to SEQ ID NO:256 or 257. In some embodiments, the polypeptide comprises an amino acid sequence with at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity (or any derivable range therein) to SEQ ID NO:256 or 257.

In some embodiments, the protein G Fab binding domain further comprises a substitution of the amino acid corresponding to position 19 of SEQ ID NO:23. In some embodiments, the substitution of the amino acid corresponding to position 19 of SEQ ID NO:3 is with a glutamic acid. For example, embodiments relate to protein G Fab binding domains of SEQ ID NOS:3-5 comprising a substitution of amino acid 19 of SEQ ID NO:3-5. In some embodiments, the substitution is of the amino acid corresponding to position 32 of SEQ ID NO:6-8. In some embodiments, the substitution of amino acid corresponding to position 32 of SEQ ID NO:6-8 is with a glutamic acid. For example, embodiments relate to protein G Fab binding domains of SEQ ID NOS:6-8 comprising a substitution of amino acid 32 of SEQ ID NO:3-5. The substitution may be a conservative, a non-conservative substation or may be any one of the known amino acids. In some embodiments, the substitution is with a glutamic acid.

In some embodiments, the polypeptides of the disclosure may further comprise an accessory molecule. Polypeptides of the disclosure, include polypeptides comprising a light chain and/or heavy chain region of a Fab, polypeptides comprising a protein G Fab binding domain and the like may include one or more accessory molecules. The accessory molecule may be a therapeutic agent, a detectable marker, a therapeutic control, a cytotoxic agent, an enzyme, a sortable tag, and the like. In some embodiments, the accessory molecule comprises an additional therapy, as described herein, such as a cytokine, a chemotherapy, a checkpoint inhibitor, an adjuvant, an antigen, a therapeutic antibody or antigen binding fragment thereof, an anti-inflammatory agent, and the like. In some embodiments, the accessory molecule comprises one or more of an antibiotic, and-inflammatory agent, anti-tumor drug, cytotoxin, and radioactive agent, and a prodrugs of a bioactive agent.

In some embodiments of the disclosure, the immune cells described herein may comprise A) i) a polypeptide comprising a protein G Fab binding domain comprising a modified isotype recognition region and further comprising a peptide spacer, a transmembrane domain, and an endodomain; or ii) a nucleic acid encoding a polypeptide comprising a protein G Fab binding domain comprising a modified isotype recognition region and further comprising a peptide spacer, a transmembrane domain, and an endodomain; and B) a polypeptide or nucleic acid encoding for a polypeptide comprising a Fab that specifically binds to a tumor antigen; and wherein the Fab comprises a kappa constant region of an antibody light chain, wherein the constant region comprises: i) a substitution/deletion of amino acids corresponding to positions 16-20 of SEQ ID NO:1 of the constant region with the amino acids LRT; or ii) a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

In some embodiments, the polypeptide may further comprises one or more linkers. The linker may be 100-150 Å. In some embodiments, the linker is less than 100 Å. In some embodiments, the linker comprises 10-20 amino acid residues. In some embodiments, the linker is at least, at most, or exactly 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 Å, or any derivable range therein. In some embodiments, the linker comprises 20-30 amino acid residues. In some embodiments, the linker comprises at least, at most, or exactly 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 amino acid residues, or any derivable range therein. In some embodiments, the linker comprises a flexible linker. In some embodiments, the linker comprises a rigid linker. In some embodiments, the linker comprises glycine and serine residues. In some embodiments, the linker comprises a linker disclosed herein.

In some embodiments, of the disclosure, the Fab and the protein G Fab binding domain have no significant binding affinity. For example, polypeptide embodiments include protein G Fab binding domains linked (either chemically or through a peptide bond to the heavy and/or light chain region of a Fab) to a Fab. It may be preferable that the Fab and protein G Fab binding domain have little to no binding affinity so that the polypeptide does not self-associate. In some embodiments, the Fab comprises the amino acid sequence of DEQLKSGT (SEQ ID NO:18) or SEELQANK (SEQ ID NO:19) at amino acid positions corresponding to positions 15-22 or SEQ ID NO:1

In specific embodiments, the polypeptide of the disclosure comprises a modified isotype recognition region of SEQ ID NO:9. In a further specific embodiment, the Fab specifically binds to a T cell surface receptor or a tumor antigen. In some embodiments, the Fab specifically binds to a T cell surface receptor and wherein the T cell surface receptor comprises CD3. In some embodiments, the Fab or antigen binding fragment specifically binds to a tumor antigen and wherein the tumor antigen comprises CD19 or CD20. In some embodiments, the polypeptide or method comprises administration of a polypeptide that binds to both CD19 and CD20. For example, the polypeptide may comprise an antigen binding domain that binds to one of CD19 or CD20 and comprise a protein G Fab binding domain that binds to an administered Fab of the other of CD19 or CD20. Accordingly, the current disclosure is useful for the novel design of bi-specific and multi-specific reagents and therapeutic molecules.

In some embodiments, the protein G Fab binding domain comprises an amino acid sequence of one of SEQ ID NO:3-8 or 256-257 or an amino acid sequence having at least 70% sequence identity to one of SEQ ID NO:3-8 or 256-257. These protein G Fab binding domains include:

(SEQ ID NO: 6)
TPAVTTYKLVINGRTLSGYTTTTAVDAATAEKVFKQYAYVHEVDGEWTYD
DATKTFTVTEKPEKL,
(SEQ ID NO: 7)
TPAVTTYKLVINGRTLSGYTTTTAVDAATAEKVFKQYAFGNGVDGEWTYD
DATKTFTVTEKPEKL,
(SEQ ID NO: 8)
TPAVTTYKLVINGRTLSGYTTTTAVDAATAEKVFKQIDMVSSVDGEWTYD
DATKTFTVTEKPEKL;
(SEQ ID NO: 257)
TPAVTTYKLVINGRTLSGYTTTTAVDAATAEEVFKQYAYVHEVDGEWTYD
DATKTFTVTEKPEKL.

In some embodiments, the protein G Fab binding domain comprises an amino acid sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity (or any derivable range therein) to one of SEQ ID NO:3-8 or 256-257.

In some embodiments, the heavy and light chain regions of the Fab are conjugated through a linker. The linker may be a peptide linker and provide conjugation through a peptide bond or the linker may be a chemical linker. Suitable linkers are described herein. In some embodiments, the light chain region is amino-proximal to the heavy chain region. In alternative embodiments, the light chain region is carboxy-proximal to the heavy chain region. In some embodiments, the heavy and light chain regions of the Fab are conjugated to the protein G Fab binding domain through a linker. In some embodiments, the protein G Fab binding domain is amino-proximal to the Fab. In some embodiments, the protein G Fab binding domain is carboxy-proximal to the Fab.

In some embodiments, the heavy and light chain regions of the Fab are linked through binding affinity and are not conjugated through a peptide bond. In some embodiments, the heavy and light chain are chemically linked.

In some embodiments, the protein G Fab binding domain is conjugated to the light chain region of the Fab through a linker. In some embodiments, the protein G Fab binding domain is conjugated to the heavy chain region of the Fab through a linker. In some embodiments, the protein G Fab binding domain is carboxy-proximal to the heavy or light chain region of the Fab. In some embodiments, the protein G Fab binding domain is amino-proximal to the heavy or light chain region of the Fab. In some embodiments, the polypeptide comprises a further antigen binding fragment, such as one or more of a single chain variable fragment (scFv), a single domain antibody, a single chain antibody, and the heavy and/or light chain of a Fab. In some embodiments, the antigen binding fragment specifically binds to a tumor antigen, an inflammatory or anti-inflammatory cytokine, a T cell surface receptor, or a cell-specific surface protein.

In some embodiments, the polypeptide comprising the peptide spacer, transmembrane domain, and endodomain is amino-proximal to the heavy and/or light chain region of the Fab. In some embodiments, the polypeptide comprising the peptide spacer, transmembrane domain, and endodomain is carboxy-proximal to the heavy and/or light chain region of the Fab. In some embodiments, the polypeptide comprising the peptide spacer, transmembrane domain, and endodomain is conjugated to the light chain region of the Fab through a linker. In some embodiments, the polypeptide comprising the peptide spacer, transmembrane domain, and endodomain is conjugated to the heavy chain region of the Fab through a linker. In some embodiments, the polypeptide comprising the peptide spacer, transmembrane domain, and endodomain is carboxy-proximal to the heavy or light chain region of the Fab. In some embodiments, the polypeptide comprising the peptide spacer, transmembrane domain, and endodomain is amino-proximal to the heavy or light chain region of the Fab.

In some embodiments, the protein G Fab binding domain is amino-proximal to the peptide spacer, transmembrane domain, and/or endodomain. In some embodiments, the protein G Fab binding domain is carboxy-proximal to the to the peptide spacer, transmembrane domain, and/or endodomain. In some embodiments, the polypeptide has the structure: X-PS-T-E or wherein X comprises the Fab or protein G binding protein, PS is the peptide spacer, T is the transmembrane domain, and E is the endodomain. In some embodiments, the polypeptide further comprises a co-stimulatory region. In some embodiments, the co-stimulatory region is between the transmembrane domain and endodomain.

In some embodiments, the Fab and/or the antigen binding fragment specifically binds to a tumor antigen, an inflammatory or anti-inflammatory cytokine, a T cell surface receptor, a microbial antigen, a bacterial antigen, or a cell-specific surface protein.

In some embodiments, the polypeptides of the disclosure may further comprise one or more Fc regions, such as at least 1, 2, 3, 4, 5, or 6 (or any derivable range therein) Fc regions. In some embodiments, the polypeptide further comprises a targeting moiety. In some embodiments, the polypeptide comprises at least two protein G Fab binding domains or at least two modified protein G Fab binding domains. In some embodiments, the polypeptide comprises at least 2, 3, 4, 5, or 6 Fab binding domains (or any derivable range therein). In some embodiments, at least one of the modified protein G Fab binding domains comprises an isotype recognition region having the following amino acid sequence: YAYVHE (SEQ ID NO:9). In some embodiments,

In some embodiments, the therapeutic cell comprises an immune cell. In some embodiments, the therapeutic cell comprises a T cell, a regulatory T cell, a natural killer T cell, or an invariant natural killer T cell, or an induced pluripotent cell. In some embodiments, the cell is a CD4+ or CD8+ T cell. In some embodiments, the cell is derived from a stem cell, such as a hematopoietic stem cell or progenitor cell. In some embodiments, the cell has been differentiated in vitro from a stem cell, such as an HSPC or an iPSC. In some embodiments, the cell is ex vivo. The cells may be autologous or non-autologous.

In some embodiments, the methods of the disclosure relate to a method is for treating cancer, an autoimmune condition, reducing an inflammatory response, a viral infection, or a microbial infection. In some embodiments, the method further comprises administering a polypeptide comprising a constant region of an antibody light chain, wherein the constant region comprises a substitution/deletion of amino acids corresponding to positions 16-20 of SEQ ID NO:1 of the constant region with the amino acids LRT. In some embodiments, the method further comprises administering a polypeptide comprising a constant region of an antibody light chain, wherein the constant region comprises a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

In some embodiments, the detection pair comprises an enzyme and detecting the detection pair comprises detecting enzymatic activity. In some embodiments, the detection pair comprises a TEM-1 β-lactamase (BL). In some embodiments, the first component of the detection pair comprises the BLF1 fragment of the TEM-1 BL. In some embodiments the second component of the detection pair comprises the BLF2 fragment of the TEM-1 BL.

In some embodiments, the first and second component of the detection pair comprise a complimentary donor and acceptor fluorophore. In some embodiments, the first Fab comprises a constant region of an antibody light chain, wherein the constant region comprises a substitution/deletion of amino acids corresponding to positions 16-20 of SEQ ID NO:1 of the constant region with the amino acids LRT. In some embodiments, the first protein G binding domain comprises an isotype recognition region having the following amino acid sequence: YAYVHE (SEQ ID NO:9). In some embodiments, the second Fab comprises a human or mouse kappa or lambda light chain. In some embodiments, the second protein G binding domain comprises an isotype recognition region having one of the following amino acid sequences: YAFGNG (SEQ ID NO:10) or IDMVSS (SEQ ID NO:11). In some embodiments, the first protein G Fab-binding domain has a higher affinity for the first Fab compared to the second Fab, and the second protein G Fab-binding domain has a higher affinity for the second Fab compared to the first Fab. In some embodiments, the first polypeptide is linked to the first detection pair through a linker and/or wherein the second polypeptide is linked to the second detection pair through a linker. In some embodiments, the first or second polypeptide further comprises one or more of Fc region(s), targeting moieties, accessory molecules, and combinations thereof.

In some embodiments, the kits of the disclosure comprise an enzyme and/or a substrate.

In some embodiments, the polypeptide comprises a variant immunogenicity region having a sequence with at least 90% homology or identity to X_{2′}VIX_{5′}GX_{7′}X_{8′}LX_{10′}X_{11′} (SEQ ID NO:81), wherein X_{2′} is L or F; X_{5′} is N, R, G, M, I, S, or L; X_{7′} is R, L, V, I, or S; X_{8′} is T or R; X_{10′} is S, W, L, G, or R; X_{11′} is L, F, or V; and wherein the variant immunogenicity region is not LVINGRTLSG (SEQ ID NO:57). In some embodiments, the variant immunogenicity region is selected from SEQ ID NOS:58-81.

In some embodiments, the polypeptide further comprises a targeting moiety. The term “targeting moiety,” as used herein, refers to species that will selectively localize in a particular tissue or region of the body. The localization is mediated by specific recognition of molecular determinants, molecular size of the targeting agent or conjugate, ionic interactions, hydrophobic interactions and the like. Other mechanisms of targeting an agent to a particular tissue or region are known to those of skill in the art. Exemplary targeting moieties include antibodies, antibody fragments (e.g. Fabs), transferrin, HS-glycoprotein, coagulation factors, serum proteins, .beta.-glycoprotein, G-CSF, GM-CSF, M-CSF, EPO and the like.

Further aspects of the disclosure relate to a fusion protein comprising a fusion between two or more polypeptides or protein G Fab binding domains described herein. Fusion of the polypeptides or protein G variants allows for binding of multiple Fab polypeptides to the fusion protein. This has the potential to make a polypeptide that has multivalency with respect to the Fab regions, and such complexes can recognize more than one epitope if different Fabs are bound to the same fusion protein. The protein G variants may be fused directly to each other or through a linker. In some embodiments, the linker comprises glycine and serine residues.

In some embodiments, the polypeptides described herein are non-naturally occurring polypeptides. In some embodiments, the polypeptide comprises post-translation modifications that are different than the polypeptide produced in its native environment. For example, the polypeptide may differ in the status of myristoylation, palmitoylation, isoprenylation or prenylation, farnesylation, geranylgeranylation, glypiation, lipoylation, phosphopantetheinylation, diphthamide formation, ethanolamine phosphoglycerol attachment, hypusine formation, acylation, acetylation, formylation, alkylation, methylation, arginylation, polyglutamylation, polyglycylation, butyrylation, glycosylation, polysialylation, malonylation, hydroxylation, iodination (e.g. of thyroglobulin), nucleotide addition such as ADP-ribosylation, oxidation, phosphate ester (O-linked) or phosphoramidate (N-linked) formation, phosphorylation, adenylylation, propionylation, pyroglutamate formation, S-glutathionylation, S-nitrosylation, S-sulfenylation, succinylation, sulfation, glycation, carbamylation, carbonylation, biotinylation, acylation of conserved lysine residues with a biotin appendage, or pegylation.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1—Basic Fab-GA1 construct. Fab can be coupled to a variety of GA1 fusions. The fusions can contain another Fab or scFv to generated a bi-specific assemblage or another protein or protein fragment. Tags or chemical moieties can to attached to GA1 to further functionalize the fusion.

FIG. 2—Interface between Protein G (green) and Fab (yellow-gray). Residues that were diversified in the phage display mutagenesis library to convert Protein G to GA1 are numbered and marked as orange spheres. Region in the Lc of Fab^{S} that was diversified to generate the affinity matured Fab^{LRT} involved residues 123-127 marked in red. The position of E123S mutation that differentiates between the Fab^{H} and Fab^{S} scaffolds is marked by a yellow sphere.

FIG. 3A-C— Affinity maturation of the Fab-Protein G interface. A). SPR sensograms showing fast on-fast off binding kinetics between Fab^{S} and the affinity matured GA1. The concentration of Fab was serially diluted 2-fold for each run starting at 100 nM B). Sensogram showing slow-dissociation kinetics for Fab^{LRT} binding to GA1. Initial concentration of Fab was 12 nM and serially diluted as in A). C). SPR kinetics for GA1 binding to Fab^{S} and Fab^{LRT}. Figure discloses SEQ ID NO: 268.

FIG. 4—Interface contacts of Fab^{LRT} with GA1 showing the interactions of the key R126 side chain. The guanidinium portion of the side chain forms a cation-π interaction with the ring of Y40 and also a H-bond with that group's main chain carbonyl. There is also a significant rearrangement of the main chain 123-127 presumably induced by the deletion of two residues in the loop.

FIG. 5—Model for components in the complementation proximity assay showing the potential fusion points between the Fabs and the linker-BL fragments. The structure of the Asf1 Fab 1-Fab 2 complex shows that the Fabs bind to the opposite faces of Asf1. In those positions it is possible to measure the direct distances between the N and C-terminal fusion points the BL fragments on GA1 bound to its respective Fab. The direct distances range from −90-140 Å. A 30-residue linker was thought to have enough reach that it would be effective in all possible combinations.

FIG. 6A-C— Establishing background levels of Beta Lactamase (BL) activity readouts. A). Different BL fragments were mixed at 1 μM concentration. Fluorescent readings were taken every 2 mins over 20 time points. No activity was observed when the individual fusion components were mixed without their complementary pair. Activity was seen at this high concentration when the component pairs were mixed together. Although at the last time points activities are similar, the 1+4 pair, shows a distinct difference from the others over the time course. B). Background activity for the complementation pair 1-4 (GA1-BLF1(1) and BLF2-GA1(4)) when mixed at varying concentrations. Readings were taken at 2 min intervals over a 1 hr incubation time frame. Data show that the signal is at background at 250 nM concentration of the pair. C). Asf1 antigen detection using different BFL combinations. Fabs 11E and 12E were mixed with BLF fragments at 250 nM concentration. 250 nM of Asf1 was then added. (−) is the signal prior to Asf1 addition, (+) after addition of antigen. BL activity was measured after 20 mins incubation at RT.

FIG. 7A-D—Analysis of binding and epitope binning using SPR. A) SPR senograms used for kinetic analysis of Mj6 and Mj20 binding to EBOV NP^{CT}. Initial concentrations (MJ6 —50 nM; MJ20—100 nM) were serially diluted by 50% between each run. B). Epitope binning experiment of Mj6 and Mj20 against EBOV NP^{CT} showing the Fabs have non-overlapping epitopes. Fab Mj20 (or Mj6) was injected as an analyte first, followed by a second injection of the other Fab. Substantial increase in RUs upon second injection indicates the two Fabs bind simultaneously. C). Binding sensograms for Z2C4 and Z2G6 binding to ZIKV MT. D). Epitope binning of the two Fabs, as described in B. Initial concentrations: Z2C4—40 nM; Z2G6—75 nM).

FIG. 8A-C—BL proximity assay results. A). Detection of EBOV NP^{CT} at different concentrations using complementary pairs: GA1(C-term)-BLF1/Mj6 and BLF2-GA1(N-term)/Mj20. Detectable signal was observed starting at 15 nM and peaking at 250 nM. Last bar shows that NP^{CT} is readily detected in the context of the full length EBOV NP at 250 nM. B). Concentration dependence of detection of full length EBOV NP. C). Concentration dependence of ZIKV MT detection using complementary pairs: GA1(C-term)-BLF1/Z2C4 and BLF2-GA1(N-term)/Z2G6. In all experiments, reactions were incubated for 20 mins at RT; a background of 200 units of substrate fluorescence was subtracted.

FIG. 9. BiTE construct. Fab^{H} recognizes Her2 extracellular domain on the antigen presenting cells (APC). The Fab is attached by a 13 residue linker to GA1 via a fusion to the C-term of its Lc. Fab^{LRT} component binds to CD3 of the T-cell receptor. This Fab contains the CDRs of either OKT3 or UTCH1.

FIG. 10A-C— The effects of the Fab^{H}(Her2)-linker-GA1-Fab^{LRT}(OKT3/UTCH1) BiTE on PBMC/SKBR3 (10:1) co-cultures. To test the effect of the BiTE, 20K SKBR3 cells were cultured on a plate overnight. 200K of PBMCs were mixed with 50 nM of the BiTE and added on the SKBR3 cells. Cell killing effect measured by LDH activity (A) and cytokine release upon T cell activation (B, C) were measured after 24 hours incubation. As a control, all the individual components of the BiTE reagents (lanes 1 and 2) and with mutant CD3 Fab^{LRT}, deficient in CD3 binding (lanes 4, 6) were tested and showed practically no effect on LDH or cytokine levels (dashed line). The CD3 activation and cell killing was observed only when both active components of the BiTE were present (lanes 3, 4, 7, 8) and with genetically linked bi-specific molecule used as a positive control for the immunological synapse formation (9). Results of representative experiments out of three (or more) are shown. Contents of lanes: 1 (GA1+ Fab^{H}(Her2)+ Fab^{LRT}(OKT3); 2 (GA1+ Fab^{H}(Her2)+ Fab^{LRT}(UTCH1), 3 (Fab^{H}(Her2)+GA1+ Fab^{LRT}(OKT3); 4 (Fab^{H}(Her2)+GA1+mutFab^{LRT}(OKT3)); 5 (Fab^{H}(Her2)+GA1+ Fab^{LRT}(UTCH1); 6 (Fab^{H}(Her2)+GA1+ mutFab^{LRT}(UTCH1); 7 (Fab^{H}(OKT3)+GA1+ Fab^{LRT}(Her2); 8 Fab^{H}(UTCH1)+GA1+Fab^{LRT}(Her2); 9 “BiTE control”: Fab^{H}(OKT3) fused to Her2 scFV.

FIG. 11A-B. A shows that at 1 hour at room temperature, PAB, there was no visible change in pGF or pGD Kappa-Fab binding capacity. B shows that at 20 hours at room temperature, PAB, there was no more than 50% loss in Fab binding capacity.

FIG. 12A-B. Results of LC scaffold GA1-affinity maturation. A). The weblogo and the list of 6 selected motifs for Lc aa 123-127 (note the deletions in position 123-124) (SEQ ID NOS 272-274, 26, 275, and 56, respectively, in order of appearance). B). Phage ELISA results for the selected Fab clones (SEQ ID NOS 26, 275, 273, 56, and 274, respectively, in order of appearance). Blue and red—10 nM pGA1 well coating, Lilac—1 mM SNAP well coating, red—100 nM pGA1 soluble GA1 competitor.

FIG. 13. Antigen-dependent BL activity of different combinations of GA1-BLF fusions and FabLRTs. The chart represents BL activity measured by the fluorescent signal in reaction mixtures 1 to 12 after 20 min at RT. Bars for reactions detecting EBOV NPCT or ZIKV MT are shown in solid black or in black stripes, respectively. The presence of the antigen is indicated on the top. The components of each reaction mixtures are shown in the table below; the numbers for the active combinations are in red. Each component was present in the reaction at 250 nM.

FIG. 14. Tumor-cell killing by bi-Fab BiTES: The effect of Her2_GA1+hUCHT1 concentration on LDH release in PMBC-SKBR3 co-cultures. 50 nM concentration corresponding to 70% killing was chosen for the further experimentation.

FIG. 15. Strategy schematics for Kunkel-based library generation for Fab LC scaffold affinity maturation. Phagemid containing Fab MJ20 with the stop codon introduced into Lc aa position 125 was subjected to Kunkel mutagenesis using NNK NNK NNT NNK NNK randomization primer (SEQ ID NO: 263) for Lc aa positions 123-127. The library of 109 clones was produced, while the theoretical diversity for this library is approx. 1.7×10⁷ variants. Figure discloses “SQLKS” as SEQ ID NO: 268.

FIG. 16A-C. Design of GA1 CAR. A) Graphical representation showing binding of protein GA1 and FAB(LRT) scaffold. B) Schematic of GA1 CAR containing protein GA1, CD8 hinge, CD28 TM, 41BB, and CD3zeta domains. C) Surface expression of GA1CAR in jurkat cells after lentivirus transduction, determined by flow cytometry.

FIG. 17A-C. Characterization of GA1CAR in jurkat cells. A) Quantification of IL2 release from supernatants containing 7O(LRT) FAB and GA1CAR cells, cultured in MBP coated plates. B) Release of IL2 by GA1CAR cells incubated for 16 hr, with different concentrations of 7O(LRT) FAB or 7O(Kappa) FAB, in MBP coated plates. C. Affinity dependent release of IL2 by GA1CAR cells; for 7O(LRT)-MBP interaction, the affinity decreases steadily with increasing maltose concentration. Maltose titration of 7O(LRT) FAB suggest less binding affinity leads to less IL2 production.

FIG. 18A-D. Characterization of GA1CAR in human CD8⁺ T cells. A) Surface expression of GA1CAR in CD8⁺T cells, determined by flow cytometry. B) IFNγ release by GA1CAR cells, but not CD8⁺T cells, when incubated with 7O(LRT) FAB and HEKMBP target cells. C) Affinity dependent release of IFNγ by GA1 CAR cells; for 7O(LRT)-MBP interaction, the affinity decreases steadily with increasing maltose concentration. Maltose titration of 7O(LRT) FAB suggest less binding affinity leads to less IFNγ production. D) IFNγ release by GA1 CAR when exposed to different FAB-antigen pairs.

FIG. 19A-B. Cell-killing of breast cancer cell-line expressing HER2 by GA1 CAR. A) Cartoon depicting the recognition of SKBR3 cancer cell by an anti-HER2 FAB(LRT)-GA1CAR pair. B) in-vitro cytotoxicity of GA1CAR towards SKBR3 cells and MDAMB468 cells, in the presence of anti-HER2 (LRT) FAB at different concentrations. Cytotoxicity was measured by the release of lactate dehydrogenase (LDH) into the cultured media after 16 hr.

FIG. 20. Targeting of cancer cells by GA1CAR and a FAB(LRT) cocktail. A) Graphical representation of GA1CAR recognizing a cancer cell by simultaneously targeting several antigens. FABs can be delivered simultaneously or sequentially depending on the condition.

FIG. 21. Alignment of light chain regions.

### DESCRIPTION OF ILLUSTRATIVE EMBODIMENTS

Engineered recombinant antibody-based reagents are rapidly supplanting traditionally derived antibodies in many cell biological applications. A particularly powerful aspect of these engineered reagents is that other modules having myriad functions can be attached to them either chemically or through molecular fusions. However, these processes can be cumbersome and do not lend themselves to high throughput applications. Consequently, the inventors have endeavored to develop a platform that can introduce multiple functionalities into a class of Fab-based affinity reagents in a “plug and play” fashion. This platform exploits the ultra-tight binding interaction between affinity matured variants of a Fab scaffold (Fab^{S}) and a domain of an immunoglobulin binding protein, protein G (GA1). GA1 is easily genetically manipulatable facilitating the ability to link these modules together like beads on a string with adjustable spacing to produce multivalent and bi-specific entities. GA1 can also be fused to other proteins or be chemically modified to engage other types of functional components. To demonstrate the utility for the Fab-GA1 platform, the inventors applied it to a detection proximity assay based on the β-lactamase (BL) split enzyme system. The Examples of the application also show the bi-specific capabilities of the module by using it in context of a Bi-specific T-cell engager (BiTE), which is a therapeutic assemblage that induces cell killing by crosslinking T-cells to cancer cells. The inventors show that GA1-Fab modules are easily engineered into potent cell killing BiTE-like assemblages and have the advantage of interchanging Fabs directed against different cell surface cancer related targets in a plug and play fashion.

### I. MODIFIED PROTEIN G FAB BINDING DOMAIN

The protein G Fab-binding domain (C-domain) may be any C domain from a protein G. Protein G is an immunoglobulin-binding protein expressed in Streptococcal bacteria. An example of a protein G is shown in SEQ ID NO:20 below:

(SEQ ID NO: 20)
EFNKYGVSDYYKNLINNAKTVEGVKDLQAQVVESAKKARISEATDGLSD
FLKSQTPAEDTVKSIELAEAKVLANRELDKYGVSDYHKNLINNAKTVEGV
KDLQAQVVESAKKARISEATDGLSDFLKSQTPAEDTVKSIELAEAKVLAN
RELDKYGVSDYYKNLINNAKTVEGVKALIDEILAALPKTDTYKLILNGKT
LKGETTTEAVDAATAEKVFKQYANDNGVDGEWTYDDATKTFTVTEKPEVI
DASELTPAVTTYKLVINGKTLKGETTTEAVDAATAEKVFKQYANDNGVDG
EWTYDDATKTFTVTEKPEVIDASELTPAVTTYKLVINGKTLKGETTTKAV
DAETAEKAFKQYANDNGVDGVWTYDDATKTFTVTEMVTEVPGDAPTEPEK
PEASIPLVPLTPATPIAKDDAKKDDTKKEDAKKPEAKKEDAKKAETLPTT
GEGSNPFFTAAALAVMAGAGALAVASKRKED.

Fab binding domain In some embodiments, the protein G is from Streptococcus. In some embodiments, the protein G variant or polypeptide comprising the modified protein G Fab binding domain comprises at least 1, 2, 3, 4, 5, 6, 7, 8, 9, or 10 (or any derivable range therein) substitutions as described herein.

In some embodiments, the Fab binding domain is in the context of all or a portion of a protein G polypeptide. In some embodiments, the polypeptide is all or a portion of a protein G described herein (i.e. SEQ ID NO:20, SEQ ID NO:21, or SEQ ID NO:22).

In some aspects, the unmodified protein G is SEQ ID NO:21:

(SEQ ID NO: 21)
MEKEKKVKYFLRKSAFGLASVSAAFLVGSTVFAVDSPIEDTPIIRNGGEL
TNLLGNSETTLALRNEESATADLTAAAVADTVAAAAAENAGAAAWEAAAA
ADALAKAKADALKEFNKYGVSDYYKNLINNAKTVEGVKDLQAQVVESAKK
ARISEATDGLSDFLKSQTPAEDTVKSIELAEAKVLANRELDKYGVSDYHK
NLINNAKTVEGVKDLQAQVVESAKKARISEATDGLSDFLKSQTPAEDTVK
SIELAEAKVLANRELDKYGVSDYYKNLINNAKTVEGVKALIDEILAALPK
TDTYKLILNGKTLKGETTTEAVDAATAEKVFKQYANDNGVDGEWTYDDAT
KTFTVTEKPEVIDASELTPAVTTYKLVINGKTLKGETTTEAVDAATAEKV
FKQYANDNGVDGEWTYDDATKTFTVTEKPEVIDASELTPAVTTYKLVING
KTLKGETTTKAVDAETAEKAFKQYANDNGVDGVWTYDDATKTFTVTEMVT
EVPGDAPTEPEKPEASIPLVPLTPATPIAKDDAKKDDTKKEDAKKPEAKK
EDAKKAETLPTTGEGSNPFFTAAALAVMAGAGALAVASKRKED.

In further embodiments, the unmodified protein G is represented by SEQ ID NO:22:

(SEQ ID NO: 22)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVINGRTLSGETTTKA
VDAETAEKAFKQYANDNGVDGVWTYDDATKTFTVTE.

Fab binding domain In some embodiments, the unmodified Fab binding domain comprises the sequence: KTLKGETTTKAVDAATAEKVFKQYANDNG (SEQ ID NO:23), KTLKGETTTEAVDAATAEKVFKQYANDNG (SEQ ID NO:24), or KTLKGETTTKAVDAETAEKAFKQYANDNG (SEQ ID NO:25).

In some embodiments, the polypeptide comprises a modified Fab binding domain comprising an amino acid sequence with at least 90% homology or identity to one of SEQ ID NOS:3-5, 31-37, or 256.

In some embodiments, the modified Fab binding domain comprises SEQ ID NO:3 or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 99, or 100% (or any derivable range therein) sequence identity to SEQ ID NO:3. In some embodiments, the modified Fab binding domain comprises SEQ ID NO:4 or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 99, or 100% (or any derivable range therein) sequence identity to SEQ ID NO:4. In some embodiments, the modified Fab binding domain comprises SEQ ID NO:5 or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 99, or 100/o (or any derivable range therein) sequence identity to SEQ ID NO:5. In some embodiments, the modified Fab binding domain comprises SEQ ID NO:31 or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 99, or 100% (or any derivable range therein) sequence identity to SEQ ID NO:31. In some embodiments, the modified Fab binding domain comprises SEQ ID NO:32 or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 99, or 100% (or any derivable range therein) sequence identity to SEQ ID NO:32. In some embodiments, the modified Fab binding domain comprises SEQ ID NO:33 or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 99, or 100% (or any derivable range therein) sequence identity to SEQ ID NO:33. In some embodiments, the modified Fab binding domain comprises SEQ ID NO:34 or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 99, or 100% (or any derivable range therein) sequence identity to SEQ ID NO:34. In some embodiments, the modified Fab binding domain comprises SEQ ID NO:35 or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 99, or 100% (or any derivable range therein) sequence identity to SEQ ID NO:35. In some embodiments, the modified Fab binding domain comprises SEQ ID NO:36 or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 99, or 100% (or any derivable range therein) sequence identity to SEQ ID NO:36. In some embodiments, the modified Fab binding domain comprises SEQ ID NO:37 or a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 99, or 100% (or any derivable range therein) sequence identity to SEQ ID NO:37.

Further protein G Fab binding domains and embodiments are described in WO/2016/061427, which is herein incorporated by reference for all purposes.

### II. VARIATIONS FROM WILD-TYPE

In some embodiments, the polypeptides described herein comprise a protein G polypeptide or portion thereof. For example, SEQ ID NO:22 describes a wild-type non-modified protein G polypeptide, and SEQ ID NO:23 describes a wild-type non-modified protein G Fab binding domain. However, there are natural variations to this polypeptide. For example, protein G from Streptococcus sp. ‘group G’ (Accession No: CAA37410) is 98% identical to SEQ ID NO:22, and varies at amino acids 78, 139, and 142 with respect to SEQ ID NO:22. GenBank Accession No: P19909 has an additional N and C-terminal sequence, has 98% identity to SEQ ID NO:22, and varies at amino acids 78, 139, and 142 with respect to SEQ ID NO:22. The N-terminal portion of P19909 also shares 91% identity to amino acids 57-185 of SEQ ID NO:22 and varies at amino acids 58-60, 65, 66, 78, 139, 142, 148, 153, 158, and 171, (or any derivable range therein) with respect to SEQ ID NO:22. Protein G from Streptococcus dysgalactiae subsp. Equisimilis (Accession No: KKC16415) shares about 94% identity with amino acids 57-185 of SEQ ID NO:22 and varies at amino acids 58-60, 65, 66, 78, 139, and 142, with respect to SEQ ID NO:22. Protein G from Streptococcus dysgalactiae (Accession No: WP_042357947) shares about 91% identity with amino acids 57-185 of SEQ ID NO:22 and varies at amino acids 58-60, 65, 66, 74, 78, 123, 126, 139, and 142 (or any derivable range therein), with respect to SEQ ID NO:22. In some instances in the variants described above, the substitution is a conservative or non-conservative substitution. Based on the natural variants known in the art, one can easily envision polypeptides of the current disclosure that share a certain percent identity to the wild-type protein G and retain Fab binding activity.

It is contemplated that the polypeptides described herein may have a sequence that has a certain percent identity to a wild-type sequence and varies with conservative substitutions. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue's side chain with one that is chemically dissimilar, such as a polar or charged amino acid for a nonpolar or uncharged amino acid, and vice versa.

A. Correlation of Structure and Functional Fab-Binding Characteristics of Protein G Polypeptides and Polypeptides Comprising Modified Protein G Fab-Binding Domains.

Based on the X-ray crystal structure of the affinity-matured protein G-A1—(SEQ ID NO:27)—Fab-Asf1 ternary complex that was determined, it is clear that the binding footprint is virtually identical to the wild-type Protein-G (Protein Data Bank entry 1IGC). Protein-G interacts with the Fab fragment through an interaction dominated by an antiparallel beta-strand configuration providing the origin of the broad isotype and species specificity (see, for example, WO/2016/061427). The engineered interface of Protein G-A1 contains several mutations that bury significant surface area at the protein interface. Serine18 of the modified A1 domain buries ˜35 Å2 while also contributing hydrogen bonds through the backbone peptide bond. The position of the modification is relative to the wild-type Fab binding domain, which is shown in SEQ ID NO:23. Therefore, Serine 18 refers to a modification to a serine at position 18 of SEQ ID NO:23. This same reference is used in the following paragraphs when discussing the structure/function of the modifications in the protein G-A1 variant (SEQ ID NO:27). The most notable mutation at the beta strand interface is Tyr20 which provides ˜70 A2 of interface for complex formation. This is achieved through substantial van der Waals interactions with the alkyl chain Lys214 from Fab CH1. In total, protein G-A1 buries ˜500 Å2 at the Fab CH1 interface, comparable to the original parent domain (550 Å2).

The largest changes within Protein-G-A1 interface occur at the C-terminal cap of the α-helix. Here, residues 40-43 (40NDNG43 (SEQ ID NO: 266)) are mutated to 40YVHE43 (SEQ ID NO: 267) in the engineered variant. The engineered helical cap provides exquisite shape complementarity to interdigitate within the alpha helix connecting beta strands 1 and 2 of CK at the heavy and light chain interface (see, for example, WO/2016/061427). Tyr40 interacts with the Ck domain, burying ˜45 Å2 primarily through contacts of its aromatic ring with the alkyl side chain of Lys126 of CK. Val41 buries roughly 90 Å2, through interactions with Ser127 of CK. His42 of Protein-G-A1 is buried at the CH1 interface where its Nε2 forms a hydrogen bond to the main chain nitrogen of the CH1 Val129 peptide bond, a hydrogen bonding interaction analogous to the polar interactions formed by Asn42 of the parent domain. The hydrogen bonding potential at this position appears to be conserved as most variants isolated at this position are either His, Asn or Gln. Glu43 projects into a groove formed by Lys126 and Glu123 to bury ˜70 Å2. Many of the newly introduced residues make extensive contact with the light chain in a manner distinct from the parent domain. As a result ˜100 Å2 of additional surface area is buried in CK, providing a small protein scaffold additional surface area to recognize its molecular partner with high affinity. Notably, the region of CK (kappa) interacting with the light chain differs significantly from the Cλ (lambda) isotype. To probe the contribution of the light chain to the affinity of the wild type and Protein-G-A1 variants, Applicants performed ELISA experiments to determine the relative affinities. The wild type domain possessed a ˜7-fold lower EC50 for CK than Cλ while Protein-G-A1 preferred CK with an EC50 ratio greater than 5000. This can be rationalized structurally as several residues that help form the distinct grooves into which the C-terminal helical cap of Protein-G-A1 interdigitates vary as a function of light chain isotype. Notably, a Lys126→Gln substitution potentially diminishes the shape complementarity of the alkyl chain projecting into the groove formed by Glu43 and Tyr40 of Protein-G-A1 as well as abolishing potential electrostatic contacts between Lys126 and Glu43. Further conformational changes within the architecture of the light chain helix likely provide subtle differences to this epitope when presented to Protein-G. Ultimately, engineering of the Fab-Protein-G interface enabled molecular recognition of a quaternary epitope created through the CH/CL interface and provides a route to endow specificity that an interaction dominated by beta-strand interaction is unlikely to achieve.

B. Phage Panning

The Protein-G-A1 helical cap library was subjected to phage panning where Fabs with unique light chain sequences were immobilized through streptavidin-biotin linkage for standard selection methods. Notably, during the phage display selection, an excess of wild type Fab, which has a kappa light chain, was added as a competitor to favor the enrichment of isotype-specific Protein-G reagents. Any binders that bind to the wild type Fab are captured and washed away, leaving only those that bind specifically to the modified light chain.

Subsequent analysis of Protein-G variants yielded clones specific to FabHS (a human 4D5 scaffold with residues PEELRTNK (SEQ ID NO:28) replacing amino acids corresponding to amino acids 15-22 of SEQ ID NO:1). Here, protein ELISA indicated minimal cross-reactivity of Protein-GHS variants C6 and C7 (YSRPHV (SEQ ID NO:29) and YAYGAV (SEQ ID NO:30), respectively) while there was robust binding to FabHS (IC50 ˜8 nM and 100 nM for C6 and C7, respectively). These results validate the Protein-G library design and selection strategy for the generation of orthogonal Protein-G reagents.

C. Multi-Valent Polypeptides Comprising Protein G Fab Binding Domains or Substituted Light Chain Regions

Multi-valency is a common feature of many biological systems that harness the simultaneous engagement of tethered ligands to multiple receptors. Polypeptides and fusion proteins of the current disclosure include multi-valent proteins made by fusing multiple protein G Fab binding domains together and/or multiple substituted light chain regions, such as the substituted Fabs described herein. Biological processes use this as a means to increase the effective affinity of weak binding ligands as well as to qualitatively modify the activity of proteins through multi-valent engagement and molecular crosslinking and to combat antigen escape. A notable example of bi-valency is an antibody, which exploits its two identical Fab antigen-binding arms to improve the affinity of antigen recognition and induce receptor crosslinking. Traditionally, antibodies have been produced by animal immunization and propagated through hybridoma technology. However, this approach has significant limitations. Since the animal strongly influences the antibody repertoire that results, there is no control over which epitopes on the antigen are targeted and immune-dominant epitopes may not be those of interest. Further, while hybridomas produce antibodies, they do not directly provide the encoding DNA. Antibody sequences drift over time and the cells die so that the antibody source is not renewable. Over the last two decades, phage display derived antibodies have become a more versatile alternative to hybridoma-based technology. The completely in vitro process offers a number of technical advantages over traditional methods including exquisite control of the selection conditions and the ability to raise antibodies against highly conserved epitopes. Applicants have helped develop novel synthetic antibody libraries based on “restricted chemical diversity” where residues within the antibody antigen binding loops of the antibody Fab fragment are enriched in amino acids typically found at the antibody paratope. Such libraries, based on the 4D5 Fab scaffold, have successfully produced affinity reagents to a wide range of targets. Furthermore, Fab fragments derived from synthetic libraries allow for the potential to move beyond the IgG format, enabling facile prokaryotic expression and further functionalization through genetic manipulation. One of the major bottlenecks in the large-scale generation and characterization of affinity reagents is their expression and purification for subsequent biophysical, structural and cell-based studies. Here, researchers select reagents based on their expression levels, stability and their ability to bind the Fab rather than the Fc.

While phage display mutagenesis is probably the most widely used directed evolution approach to generate antibody-based affinity reagents, yeast display and ribosome display methods are also viable approaches. Antibody fragments can take different forms than Fabs, but ultimately to reformat them into IgG molecules if desired, they have to be converted into Fabs as part of the process. Thus, in performing the display selections it can be more efficient to use the Fab scaffold. A further advantage is that Fab domains are generally much more stable than other forms, for instance the single chain version of the variable heavy chains (scFv). Because most of the recombinant methods to generate antibody like molecules will likely involve engineering Fab domains at some level, there is a need to develop better methods to purify Fabs in ways that do not compromise their structural integrity and to eliminate unwanted degradation products that are inherent in their expression.

Linking together Protein-G binding domains and substituted Fab polypeptides containing specially engineered properties could produce molecules that bind multiple copies of an antibody Fab or a molecule that can interact with different antigens. These constructs could capitalize on the resulting multi-valency to perform myriad new binding functions beyond those available to natural antibodies. This is because the two Fab arms of the Y-shaped antibody scaffold have significant structure limitations in how they are able to jointly present their binding paratopes toward their molecular targets. The multi-valent Protein-G constructs presumably would not have similar constraints since the linker regions between the engineered binding domains can be adjusted for length, flexibility and composition. Thus, this type of construct allows for the facile generation of a range of multivalent scaffolds where oligomeric state, specificity, linker length and geometric arrangement can be predictably controlled. Such scaffolds will serve as powerful reagents for applications where simultaneous engagement of multiple binding sites can provide enhancements in affinity and activity.

An area of active research in the biopharmaceutical industry is the engineering of bi-specific antibodies where the two Fab arms recognize different antigens (Speiss et al., 2015). This engineering involves introducing multiple mutations into the antibody scaffold and is costly and not optimally efficient. In this regard, Protein-G can be co-engineered with Fab fragments to produce molecules with multiple specificities with much more versatility than can be achieved using an antibody scaffold since many copies of the Fabs with different specificities can be linked together to combine the attributes of multi-specificity and valency in the same molecule. A further functional advance could be to introduce these multi-valent/specificity Protein-G chains into Fc frameworks thereby producing an engineered IgG that has the ability to bind multiple copies of a desired Fab to enhance avidity over what is possible with just two Fab arms. This concept can be extended by matching the Protein-G specificity to Fabs that recognize different binding partners thereby producing an IgG variant with multi-valent and bi-specific characteristics. Previously, no strategy had been proposed to enable facile control over both valency and specificity of multivalent antibody constructs.

Antibodies exploit multi-valency through naturally occurring formats including the IgG (bivalent), IgA (tetravalent) and IgM (decavalent). Here, the ability to simultaneously engage multiple binding sites through a single molecule enables the potential for enhanced affinity and activity. Synthetic antibody constructs are typically in the IgG format and further engineering to alter the Fab valency is generally difficult due to the complicated architecture of the IgG. Engineered Protein-G variants provide an alternative avenue for controlling multi-valency where the IBP can readily be produced in various oligomeric formats in high yield. Here, Protein-G can create large, controlled multi-valent constructs where Fabs are tethered through either non-covalent or covalent crosslinking. Importantly, the Protein-G construct can be controlled in a highly facile manner through introduction of defined linker lengths and oligomeric formats. Such constructs will be useful for the generation of high-capacity purification resin and the exploitation of antibody affinity and activity through multivalent affinity enhancement.

Here Applicants generated multivalent constructs through Golden Gate cloning where fragments are assembled through small, DNA overhangs created by type IIs restriction enzymes (enzymes which cut distal to the sequence they recognize). (Engler and Marillonnet. 2013). Through the generation of specific overhangs, one can rapidly assemble repetitive fragments of DNA, controlling valency, specificity and order of Protein-G molecules. Using this strategy, Applicants were able to generate Protein-G-A1 constructs ranging from a dimer to decamer using repeats of the Gly4Ser linker. These variants were readily purified to homogeneity using standard IMAC purification procedures. Notably, all variants expressed well (>5 mg/ml) in standard shake-flask expression methods. Importantly, this cloning method enables the linker length to be readily altered though modifying the fragments used for assembly. Given the high solubility of Protein-G there would be extreme flexibility in our choice of both linker length and composition.

D. Development of Bi-Specific Antibody Reagents Comprising Modified Protein G Fab Binding Domains

Applicants hypothesized a bi-specific Protein-G construct comprised of modified protein G Fab binding domains with different isotype-specificities will enable the simultaneous engagement of two different protein antigens. To demonstrate the utility of such an approach, Applicants used ELISA where antigen 1 (yeast Anti-silencing factor 1) was immobilized on a Maxisorp plate coated with neutravidin. Subsequently, a mixture of Protein-G-A1-Protein-G-HS, FabHS (specific to yAsf1) and FabK (specific to RNA-binding protein U1A) were added in stoichiometric amounts. After a period of incubation (˜15 min) and washing, U1A was titrated at concentrations of 0-250 nM. Subsequent binding of U1A was detected by anti-FLAG-HRP which detected an epitope tag on U1A. The ELISA data demonstrate titratable, saturable binding of U1A only when all reagents are added to the ELISA well indicating the Protein-G-A1-Protein-G-HS fusion allows for the simultaneous engagement of multiple, specific binding partners. Such a reagent should enable the development of facile production of multivalent constructs for rapid assessment of multispecific affinity and activity enhancement.

E. Covalent Crosslinking of Polypeptides Comprising Modified Protein G Fab Binding Domains to Fabs:

While multivalent tethers of Protein-G enable the enhancement of affinity and activity, in some instances it may be desirable to create covalent constructs ensuring the stoichiometry of the complex. Inspection of the Protein-G-A1-Fab complex structure indicated several positions in these two molecules where introduction of cysteine residues might enable covalent crosslinking through the generation of disulfide bonds between Protein-G-A1 and the Fab. Here, several positions within the anti-parallel beta-strand interaction of the complex are within a feasible distance (Cβ-Cβ distances ˜5 Å) to enable covalent crosslinking. These pairs include: Protein-G16 and FabCK221, Protein-G-A118 and FabCK220, Protein-G20 and FabCK218 and Protein-G22 and FabCK216. The generation of covalent Fab-Protein-G constructs enables the exploitation of Protein-G multivalent scaffolds when Fab and Protein G are at concentrations below that typically required to form appreciable complex (sub-nanomolar).

### III. FAB POLYPEPTIDES

The Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature. Typically, a Fab polypeptide will be produced recombinantly and will be based on the known sequence of the variable regions of the light and heavy chains of an antibody. The isolation, production, and sequencing of antibodies is known in the art.

Proteins-A and G are multi-specific proteins that are unique among the IBPs in their ability to bind to the Fc domain of the IgG, as well as the fragment antibody-binding (Fab) domain. The Fab domain is a critical portion of the antibody since it confers the antibody's antigen specificity and its binding capacity. Fab fragments are used in myriad applications and have advantages over traditional antibodies derived from animal sources because they can be generated by directed evolution processes providing for the introduction of customized properties.

Protein-G binds to the constant domain of the Fab portion of the IgG through its interaction with the CH1 domain, a highly conserved domain across many isotypes and species. (Derrick and Wigley, 1992). Because Protein-G binds to a section of the Fab that is highly conserved across all antibodies, it has the potential to be a more effective affinity reagent than Protein-A. However, the low affinity of the natural domain (KD ˜low μM) has thus far limited the usage of Protein-G as an affinity reagent compared to Protein-A (10 nM).

While Protein-A is the industry standard today, it is generally recognized that Fab antibody purification using Protein-A resin suffers from several technical issues. Methods to release efficiently the antibody from the Protein-A resin require wash steps at low pH (˜pH 2). These conditions can have deleterious effects on the structural integrity of some antibodies, which can lead to loss of function. Also, at these pHs some a small fraction of the Protein-A can leech off the column and effectively contaminate the antibody sample being purified. Further, during expression in cell culture or bacteria, some antibodies can get proteolytically clipped making them less effective. These clips are mainly in the Fab CH1 domain and thus, Protein-A binding cannot discriminate between the desired full-length form of the antibody and the degradation products. Removing these products requires a further ion-exchange purification step. Conversely, since Protein-G binds the Fab CH1 domain, it can readily discriminate between the full-length unprocessed molecule from the degradation forms since it will only bind the unprocessed Fab. This results in a clean one-step purification process.

To exploit the potential advantages of Protein-G to make it practical for therapeutic purposes, purification the inventors have engineered a protein G Fab binding domain that binds to a substituted Fab with ultra-high affinity, but has minimal affinity to endogenously produced antibodies. Thus, the polypeptides comprising the protein G Fab binding domains of the disclosure can be administered therapeutically without the undesired effect of binding to endogenous antibodies that are circulating in the body.

Exemplary Fab embodiments are shown in the table below:

### Herceptin Fab Scaffold Examples

| | | SEQ |
|---|---|---|
| | | ID |
| Description | Sequence | NO |
| MJ6 EBOV LRT- | SDIQMTQSPSSLSASVGDRVTITCRASQSVSSAVAWYQQK | 184 |
| Fab Light chain | PGKAPKLLIYSASSLYSGVPSRFSGSRSGTDFTLTISSLQPE | |
| (variable region | DFATYYCQQYSYSLVTFGQGTKVEIKrtvaapsvfifppsdl | |
| is in upper case; | rtgtasvvcllnnfypreakvqwkvdnalqsgnsqesvteqd | |
| constant region | skdstyslsstltlskadyekhkvyacevthqglsspvtksf | |
| is in lower case) | nrgec | |
| MJ6 EBOV LRT- | EISEVQLVESGGGLVQPGGSLRLSCAASGFNVYYYYIHWV | 185 |
| Fab Heavy chain | RQAPGKGLEWVASISPYYGYTSYADSVKGRFTISADTSKN | |
| (variable region | TAYLQMNSLRAEDTAVYYCARWSYDQSMSYKSGMDYW | |
| is in upper case; | GQGTLVTVSSastkgpsvfplapsskstsggtaalgclvkdy | |
| constant region | fpepvtvswnsgaltsgvhtfpavlqssglyslssvvtvpss | |
| is in lower case) | slgtqtyicnvnhkpsntkvdkkvepkscdktht | |
| MJ6 EBOV | QSVSSAVA | 186 |
| LCDR1 | | |
| MJ6 EBOV | YSASSLYS | 187 |
| LCDR2 | | |
| MJ6 EBOV | YSYSLV | 188 |
| LCDR3 | | |
| MJ6 EBOV | VYYYYI | 189 |
| HCDR1 | | |
| MJ6 EBOV | SISPYYGY | 190 |
| HCDR2 | | |
| MJ6 EBOV | WSYDQSMSYKSGM | 191 |
| HCDR3 | | |
| MJ20 RESTV | SDIQMTQSPSSLSASVGDRVTITCRASQSVSSAVAWYQQK | 192 |
| LRT-Fab | PGKAPKLLIYSASSLYSGVPSRFSGSRSGTDFTLTISSLQPE | |
| Light chain | DFATYYCQQSSSSLITFGQGTKVEIKrtvaapsvfifppsdl | |
| (variable region | rtgtasvvcllnnfypreakvqwkvdnalqsgnsqesvteqd | |
| is in upper case; | skdstyslsstltlskadyekhkvyacevthqglsspvtksf | |
| constant region | nrgec | |
| is in lower case) | | |
| MJ20 RESTV | EISEVQLVESGGGLVQPGGSLRLSCAASGFNISYSSIHWVR | 193 |
| LRT-Fab | QAPGKGLEWVASIYSYSGYTSYADSVKGRFTISADTSKNT | |
| Heavy chain | AYLQMNSLRAEDTAVYYCARSYWYHVGSWHYTGMDY | |
| (variable region | WGQGTLVTVSSastkgpsvfplapsskstsggtaalgclvkd | |
| is in upper case; | yfpepvtvswnsgaltsgvhtfpavlqssglyslssvvtvps | |
| constant region | sslgtqtyicnvnhkpsntkvdkkvepkscdktht | |
| is in lower case) | | |
| MJ20 RESTV | QSVSSAVA | 194 |
| LCDR1 | | |
| MJ20 RESTV | YSASSLYS | 195 |
| LCDR2 | | |
| MJ20 RESTV | SSSSLI | 196 |
| LCDR3 | | |
| MJ20 RESTV | ISYSSI | 197 |
| HCDR1 | | |
| MJ20 RESTV | SIYSYSGY | 198 |
| HCDR2 | | |
| MJ20 RESTV | SYWYHVGSWHYTGM | 199 |
| HCDR3 | | |
| Z2C4 ZIKV LRT- | SDIQMTQSPSSLSASVGDRVTITCRASQSVSSAVAWYQQK | 200 |
| Fab | PGKAPKLLIYSASSLYSGVPSRFSGSRSGTDFTLTISSLQPE | |
| Light chain | DFATYYCQQWYDSLITFGQGTKVEIKrtvaapsvfifppsdl | |
| (variable region | rtgtasvvcllnnfypreakvqwkvdnalqsgnsqesvteqd | |
| is in upper case; | skdstyslsstltlskadyekhkvyacevthqglsspvtksf | |
| constant region | nrgec | |
| is in lower case) | | |
| Z2C4 ZIKV LRT- | EISEVQLVESGGGLVQPGGSLRLSCAASGFNVYYSSIHWV | 201 |
| Fab | RQAPGKGLEWVAYIYPSSGSTYYADSVKGRFTISADTSKN | |
| Heavy chain | TAYLQMNSLRAEDTAVYYCARSWSPYGMDYWGQGTLVT | |
| (variable region | VSSastkgpsvfplapsskstsggtaalgclvkdyfpepvtv | |
| is in upper case; | swnsgaltsgvhtfpavlqssglyslssvvtvpssslgtqty | |
| constant region | icnvnhkpsntkvdkkvepkscdktht | |
| is in lower case) | | |
| Z2C4 ZIKV | QSVSSAVA | 202 |
| LCDR1 | | |
| Z2C4 ZIKV | YSASSLYS | 203 |
| LCDR2 | | |
| Z2C4 ZIKV | WYDSLI | 204 |
| LCDR3 | | |
| Z2C4 ZIKV | VYYSSI | 205 |
| HCDR1 | | |
| Z2C4 ZIKV | YIYPSSGS | 206 |
| HCDR2 | | |
| Z2C4 ZIKV | SWSPYGM | 207 |
| HCDR3 | | |
| Z2G6 ZIKV LRT- | SDIQMTQSPSSLSASVGDRVTITCRASQSVSSAVAWYQQK | 208 |
| Fab; Light chain | PGKAPKLLIYSASSLYSGVPSRFSGSRSGTDFTLTISSLQPE | |
| (variable region | DFATYYCQQSWSSLFTFGQGTKVEIKrtvaapsvfifppsdl | |
| is in upper case; | rtgtasvvcllnnfypreakvqwkvdnalqsgnsqesvteqd | |
| constant region | skdstyslsstltlskadyekhkvyacevthqglsspvtksf | |
| is in lower case) | nrgec | |
| Z2G6 ZIKV LRT- | EISEVQLVESGGGLVQPGGSLRLSCAASGFNVYYYYIHWV | 209 |
| Fab; Heavy chain | RQAPGKGLEWVASIYSYSGYTYYADSVKGRFTISADTSKN | |
| (variable region | TAYLQMNSLRAEDTAVYYCAREGVWWEDEFYPGLDYW | |
| is in upper case; | GQGTLVTVSSastkgpsvfplapsskstsggtaalgclvkdy | |
| constant region | fpepvtvswnsgaltsgvhtfpavlqssglyslssvvtvpss | |
| is in lower case) | slgtqtyicnvnhkpsntkvdkkvepkscdktht | |
| Z2G6 ZIKV | QSVSSAVA | 210 |
| LCDR1 | | |
| Z2G6 ZIKV | YSASSLYS | 211 |
| LCDR2 | | |
| Z2G6 ZIKV | SWSSLF | 212 |
| LCDR3 | | |
| Z2G6 ZIKV | VYYYYI | 213 |
| HCDR1 | | |
| Z2G6 ZIKV | SIYSYSGY | 214 |
| HCDR2 | | |
| Z2G6 ZIKV | EGVWWEDEFYPGL | 215 |
| HCDR3 | | |
| E11 Asf1 LRT- | SDIQMTQSPSSLSASVGDRVTITCRASQSVSSAVAWYQQK | 216 |
| Fab; Light chain | PGKAPKLLIYSASSLYSGVPSRFSGSRSGTDFTLTISSLQPE | |
| (variable region | DFATYYCQQSSDDPITFGQGTKVEIKrtvaapsvfifppsdl | |
| is in upper case; | rtgtasvvcllnnfypreakvqwkvdnalqsgnsqesvteqd | |
| constant region | skdstyslsstltlskadyekhkvyacevthqglsspvtksf | |
| is in lower case) | nrgec | |
| E11 Asf1 LRT- | EISEVQLVESGGGLVQPGGSLRLSCAASGFNISYSSIHWVR | 217 |
| Fab; Heavy chain | QAPGKGLEWVASISSYYGSTYYADSVKGRFTISADTSKNT | |
| (variable region | AYLQMNSLRAEDTAVYYCARSRGQASWDYWGQGTLVT | |
| is in upper case; | VSSastkgpsvfplapsskstsggtaalgclvkdyfpepvtv | |
| constant region | swnsgaltsgvhtfpavlqssglyslssvvtvpssslgtqty | |
| is in lower case) | icnvnhkpsntkvdkkvepkscdktht | |
| E11 Asf1 LCDR1 | QSVSSAVA | 218 |
| E11 Asf1 LCDR2 | YSASSLYS | 219 |
| E11 Asf1 LCDR3 | SWSSLF | 220 |
| E11 Asf1 HCDR1 | ISYSSI | 221 |
| E11 Asf1 HCDR2 | SISSYYGS | 222 |
| E11 Asf1 HCDR3 | SRGQASW | 223 |
| hOKT3(S)_LRT; | SDIQMTQSPSSLSASVGDRVTITCSASSSVSYMNWYQQKP | 224 |
| Light chain | GKAPKRWIYDTSKLASGVPSRFSGSGSGTDYTLTISSLQPE | |
| (variable region | DFATYYCQQWSSNPFTFGQGTKVEIKrtvaapsvfifppsdl | |
| is in upper case; | rtgtasvvcllnnfypreakvqwkvdnalqsgnsqesvteqd | |
| constant region | skdstyslsstltlskadyekhkvyacevthqglsspvtks | |
| is in lower case) | fnrgec | |
| hOKT3(S)_LRT; | EISEVQLVESGGGLVQPGGSLRLSCAASGYTFTRYTMHW | 225 |
| Heavy chain | VRQAPGKGLEWIGYINPSRGYTNYNQKFKDKATISTDKS | |
| (variable region | KNTAYLQMNSLRAEDTAVYYCARYYDDHYSLDYWGQG | |
| is in upper case; | TLVTVSSastkgpsvfplapsskstsggtaalgclvkdyfpe | |
| constant region | pvtvswnsgaltsgvhtfpavlqssglyslssvvtvpssslg | |
| is in lower case) | tqtyicnvnhkpsntkvdkkvepkscdktht | |
| hOKT3 LCDR1 | SSVSYMN | 226 |
| hOKT3 LCDR2 | YDTSKLAS | 227 |
| hOKT3 LCDR3 | WSSNP | 228 |
| hOKT3 HCDR1 | TFTRYTMHW | 229 |
| hOKT3 HCDR2 | IGYINPSRGYTNYNQKFKDKA | 230 |
| hOKT3 HCDR3 | YYDDHYSL | 231 |
| MutHcR58D | SDIQMTQSPSSLSASVGDRVTITCSASSSVSYMNWYQQKP | 232 |
| hOKT3(S)_LRT; | GKAPKRWIYDTSKLASGVPSRFSGSGSGTDYTLTISSLQPE | |
| Light chain | DFATYYCQQWSSNPFTFGQGTKVEIKrtvaapsvfifppsdl | |
| (variable region | rtgtasvvcllnnfypreakvqwkvdnalqsgnsqesvteqd | |
| is in upper case; | skdstyslsstltlskadyekhkvyacevthqglsspvtksf | |
| constant region | nrgec | |
| is in lower case) | | |
| MutHcR58D | EISEVQLVESGGGLVQPGGSLRLSCAASGYTFTRYTMHW | 233 |
| hOKT3(S)_LRT; | VRQAPGKGLEWIGYINPSDGYTNYNQKFKDKATISTDKS | |
| Heavy chain | KNTAYLQMNSLRAEDTAVYYCARYYDDHYSLDYWGQG | |
| (variable region | TLVTVSSastkgpsvfplapsskstsggtaalgclvkdyfpe | |
| is in upper case; | pvtvswnsgaltsgvhtfpavlqssglyslssvvtvpsssl | |
| constant region | gtqtyicnvnhkpsntkvdkkvepkscdktht | |
| is in lower case) | | |
| MutHcR58D | SSVSYMN | 234 |
| hOKT3 LCDR1 | | |
| MutHcR58D | YDTSKLAS | 235 |
| hOKT3 LCDR2 | | |
| MutHcR58D | WSSNP | 236 |
| hOKT3 LCDR3 | | |
| MutHcR58D | TFTRYTMHW | 237 |
| hOKT3 HCDR1 | | |
| MutHcR58D | IGYINPSDGYTNYNQKFKDKA | 238 |
| hOKT3 HCDR2 | | |
| MutHcR58D | YYDDHYSL | 239 |
| hOKT3 HCDR3 | | |
| hOKT3(S)_E_13 | SDIQMTQSPSSLSASVGDRVTITCSASSSVSYMNWYQQKP | 240 |
| _GA1; Light | GKAPKRWIYDTSKLASGVPSRFSGSGSGTDYTLTISSLQPE | |
| chain (variable | DFATYYCQQWSSNPFTFGQGTKVEIKrtvaapsvfifppsde | |
| region is in upper | qlksgtasvvcllnnfypreakvqwkvdnalqsgnsqesvte | |
| case; constant | qdskdstyslsstltlskadyekhkvyacevthqglsspvtk | |
| region is in lower | sfnrgec | |
| case) | | |
| hOKT3(S)_E_13 | Heavy chain: | 241 |
| _GA1; Heavy | EISEVQLVESGGGLVQPGGSLRLSCAASGYTFTRYTMHW | |
| chain (variable | VRQAPGKGLEWIGYINPSRGYTNYNQKFKDKATISTDKS | |
| region is in upper | KNTAYLQMNSLRAEDTAVYYCARYYDDHYSLDYWGQG | |
| case; constant | TLVTVSSastkgpsvfplapsskstsggtaalgclvkdyfpe | |
| region is in lower | pvtvswnsgaltsgvhtfpavlqssglyslssvvtvpssslg | |
| case) | tqtyicnvnhkpsntkvdkkvepkscdkthtggsgsagsgga | |
| | gatpavttyklvingrtlsgyttttavdaataekvfkqyayv | |
| | hevdgewtyddatktftvtekpekl | |
| hOKT3 LCDR1 | SSVSYMN | 242 |
| hOKT3 LCDR2 | DTSKLAS | 243 |
| hOKT3 LCDR3 | WSSNPF | 244 |
| hOKT3 HCDR1 | TFTRYTMHW | 245 |
| hOKT3 HCDR2 | IGYINPSRGYTNYNQKFKDKA | 246 |
| hOKT3 HCDR3 | YYDDHYSL | 247 |
| hUCHT1_LRT | SDIQMTQSPSSLSASVGDRVTITCSASQDIRNYLNWYQQK | 248 |
| Light chain | PGKAPKRWIYYTSRLHSGVPSRFSGSGSGTDYTLTISSLQP | |
| (variable region | EDFATYYCQQGNTLPWTFGQGTKVEIKrtvaapsvfifppsd | |
| is in upper case; | lrtgtasvvcllnnfypreakvqwkvdnalqsgnsqesvteq | |
| constant region | dskdstyslsstltlskadyekhkvyacevthqglsspvtks | |
| is in lower case) | fnrgec | |
| hUCHT1_LRT | EISEVQLVESGGGLVQPGGSLRLSCAASGFNFTGYTIHWV | 249 |
| Heavy chain | RQAPGKGLEWMGLINPYKGVSTYNQKFKDKATISTDKSK | |
| (variable region | NTAYLQMNSLRAEDTAVYYCARSGYYGDSDWYFDYWG | |
| is in upper case; | QGTLVTVSSastkgpsvfplapsskstsggtaalgclvkdyf | |
| constant region | pepvtvswnsgaltsgvhtfpavlqssglyslssvvtvpsss | |
| is in lower case) | lgtqtyicnvnhkpsntkvdkkvepkscdktht | |
| hUCHT1 LCDR1 | QDIRNYLN | 250 |
| hUCHT1 LCDR2 | IYYTSRLHS | 251 |
| hUCHT1 LCDR3 | GNTLPW | 252 |
| hUCHT1 HCDR1 | NFTGYTIHW | 253 |
| hUCHT1 HCDR2 | MGLINPYKGVSTYNQKFKDKA | 254 |
| hUCHT1 HCDR3 | SGYYGDSDWYF | 255 |

H- human; M- murine

Embodiments of the disclosure relate to polypeptides comprising a variable region, wherein the variable region comprises a heavy chain variable region comprising HCDR1, HCDR2, and HCDR3 and a light chain variable region comprising LCDR1, LCDR2, and LCDR3. The CDR regions include those described above. Thus, the current disclosure relates to polypeptides, Fabs, and/or an antibody comprising: 1) a light chain variable region comprising LCDR1, LCDR2, and LCDR3 of SEQ ID NOS:186-188 and a heavy chain variable region comprising HCDR1, HCDR2, and HCDR3 of SEQ ID NOS:189-191; 2) a light chain variable region comprising LCDR1, LCDR2, and LCDR3 of SEQ ID NOS:194-196 and a heavy chain variable region comprising HCDR1, HCDR2, and HCDR3 of SEQ ID NOS:197-199; 3) a light chain variable region comprising LCDR1, LCDR2, and LCDR3 of SEQ ID NOS:202-204 and a heavy chain variable region comprising HCDR1, HCDR2, and HCDR3 of SEQ ID NOS:205-207; 4) a light chain variable region comprising LCDR1, LCDR2, and LCDR3 of SEQ ID NOS:210-212 and a heavy chain variable region comprising HCDR1, HCDR2, and HCDR3 of SEQ ID NOS:213-215; 5) a light chain variable region comprising LCDR1, LCDR2, and LCDR3 of SEQ ID NOS:218-220 and a heavy chain variable region comprising HCDR1, HCDR2, and HCDR3 of SEQ ID NOS:221-223; 6) a light chain variable region comprising LCDR1, LCDR2, and LCDR3 of SEQ ID NOS:226-228 and a heavy chain variable region comprising HCDR1, HCDR2, and HCDR3 of SEQ ID NOS:229-231; 7) a light chain variable region comprising LCDR1, LCDR2, and LCDR3 of SEQ ID NOS:234-236 and a heavy chain variable region comprising HCDR1, HCDR2, and HCDR3 of SEQ ID NOS:237-239; 8) a light chain variable region comprising LCDR1, LCDR2, and LCDR3 of SEQ ID NOS:242-244 and a heavy chain variable region comprising HCDR1, HCDR2, and HCDR3 of SEQ ID NOS:245-247; or 9) a light chain variable region comprising LCDR1, LCDR2, and LCDR3 of SEQ ID NOS:250-252 and a heavy chain variable region comprising HCDR1, HCDR2, and HCDR3 of SEQ ID NOS:253-255.

Further embodiments of the disclosure relate to polypeptides comprising a variable region, wherein the variable region comprises a heavy chain variable region comprising HCDR1, HCDR2, and HCDR3. The CDR regions include those described above. Thus, the current disclosure relates to polypeptides, Fabs, and/or an antibodies comprising: 1) a heavy chain variable region comprising HCDR1, HCDR2, and HCDR3 of SEQ ID NOS:189-191; 2) a heavy chain variable region comprising HCDR1, HCDR2, and HCDR3 of SEQ ID NOS:197-199; 3) a heavy chain variable region comprising HCDR1, HCDR2, and HCDR3 of SEQ ID NOS:205-207; 4) a heavy chain variable region comprising HCDR1, HCDR2, and HCDR3 of SEQ ID NOS:213-215; 5) a heavy chain variable region comprising HCDR1, HCDR2, and HCDR3 of SEQ ID NOS:221-223; 6) a heavy chain variable region comprising HCDR1, HCDR2, and HCDR3 of SEQ ID NOS:229-231; 7) a heavy chain variable region comprising HCDR1, HCDR2, and HCDR3 of SEQ ID NOS:237-239; 8) a heavy chain variable region comprising HCDR1, HCDR2, and HCDR3 of SEQ ID NOS:245-247; or 9) a heavy chain variable region comprising HCDR1, HCDR2, and HCDR3 of SEQ ID NOS:253-255.

Further embodiments of the disclosure relate to polypeptides comprising a variable region, wherein the variable region comprises a light chain variable region comprising LCDR1, LCDR2, and LCDR3. The CDR regions include those described above. Thus, the current disclosure relates to polypeptides, Fabs, and/or an antibodies comprising: 1) a light chain variable region comprising LCDR1, LCDR2, and LCDR3 of SEQ ID NOS:186-188; 2) a light chain variable region comprising LCDR1, LCDR2, and LCDR3 of SEQ ID NOS:194-196; 3) a light chain variable region comprising LCDR1, LCDR2, and LCDR3 of SEQ ID NOS:202-204; 4) a light chain variable region comprising LCDR1, LCDR2, and LCDR3 of SEQ ID NOS:210-212; 5) a light chain variable region comprising LCDR1, LCDR2, and LCDR3 of SEQ ID NOS:218-220; 6) a light chain variable region comprising LCDR1, LCDR2, and LCDR3 of SEQ ID NOS:226-228; 7) a light chain variable region comprising LCDR1, LCDR2, and LCDR3 of SEQ ID NOS:234-236; 8) a light chain variable region comprising LCDR1, LCDR2, and LCDR3 of SEQ ID NOS:242-244; or 9) a light chain variable region comprising LCDR1, LCDR2, and LCDR3 of SEQ ID NOS:250-252.

Further embodiments of the disclosure relate to polypeptides comprising a light and heavy chain region, wherein the light chain and heavy chain comprise an amino acid sequence of: 1) SEQ ID NO:184 and 185, respectively; 2) SEQ ID NO:192 and 193, respectively; 3) SEQ ID NO:200 and 201, respectively; 4) SEQ ID NO:208 and 209, respectively; 5) SEQ ID NO:216 and 217, respectively; 6) SEQ ID NO:224 and 225, respectively; 7) SEQ ID NO:232 and 233, respectively; 8) SEQ ID NO:240 and 241, respectively; or 9) SEQ ID NO:248 and 249, respectively.

### IV. FUNCTIONAL ANTIBODY FRAGMENTS AND ANTIGEN-BINDING FRAGMENTS

A. Antigen-Binding Fragments

Certain aspects relate to antibody fragments, such as antibody fragments that bind to antigen. The term antigen-binding fragments include fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, N Y (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two VH and VL domains, and can further include all or part of the two CL and CH1 domains. In some embodiments of the disclosure, the Fab is a Fab′ or a F(ab′)2 fragment.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

B. Fragment Crystallizable Region, Fc

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

### V. POLYPEPTIDES OF THE DISCLOSURE

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID Nos:1-275.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID Nos:1-275.

In some embodiments, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390,391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:1-275.

In some embodiments, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs:1-275 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOs:1-275.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOs:1-275 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOs:1-275.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

A. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The substitution in the variant polypeptide may be a substitution of a histidine, isoleucine, leucine, lysine, methionine, phenylalanine, threonine, tryptophan, valine, arginine, cysteine, glutamine, glycine, proline, tyrosine, alanine, aspartic acid, asparagine, glutamic acid, serine, selenocysteine, or pyrrolysine for a different amino acid, such as for a histidine, isoleucine, leucine, lysine, methionine, phenylalanine, threonine, tryptophan, valine, arginine, cysteine, glutamine, glycine, proline, tyrosine, alanine, aspartic acid, asparagine, glutamic acid, serine, selenocysteine, or pyrrolysine.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants, or combinations thereof. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more (or any derivable range therein) non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more (or any derivable range therein) substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

B. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within +1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within +2 are included, in other embodiments, those which are within +1 are included, and in still other embodiments, those within 0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### VI. DETECTABLE LABELS

In some aspects of this disclosure, it will be useful to detectably or therapeutically label the Fab polypeptide or protein G Fab binding domain. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### VII. DETECTION PAIRS

Detection pairs include two complementary proteins, nucleic acids, or molecules that upon interaction produces a readout such as an enzymatic activity or colorimetric or fluorescent signal. Protein detection pairs can be two halves of an enzyme that upon interaction become one active enzyme. Enzymes include beta-lactamase, dihydrofolate reductase, focal adhesion kinase, horseradish peroxidase, Gal4, beta-galactosidase, luciferase or tobacco etch virus protease. Protein detection pairs can also be two halves of a fluorescent protein that upon interaction produce a fluorescence signal. Fluorescent proteins include green fluorescent proteins. Detection pairs can also comprise fluorophores or chromophores which involve a donor and acceptor whose proximity generates a detectable signal of fluorescence of phosphorescence.

### VIII. NUCLEIC ACIDS

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% (or any derivable range therein) or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more (or any derivable range therein) nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

D. Vectors

Polypeptides described herein may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). In addition to encoding a variant SpA polypeptide the vector can encode other polypeptide sequences such as a one or more other bacterial peptide, a tag, or an immunogenicity enhancing peptide. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

E. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

Various elements/promoters may be employed in the context of the present disclosure to regulate the expression of a gene. Examples of such inducible elements, which are regions of a nucleic acid sequence that can be activated in response to a specific stimulus, include but are not limited to Immunoglobulin Heavy Chain, Immunoglobulin Light Chain, T Cell Receptor, HLA DQ and/or DQ, Interferon, Interleukin-2, Interleukin-2, MHC Class II, MHC Class II HLA-DR, Actin, Muscle Creatine Kinase (MCK), Prealbumin (Transthyretin), Elastase I, Metallothionein (MTII), Collagenase, Albumin, Fetoprotein, γ-Globin, Globin, c-fos, c-Ha-Ras, Insulin, Neural Cell Adhesion Molecule (NCAM), 1-Antitrypain, H2B (TH2B) Histone, Mouse and/or Type I Collagen, Glucose-Regulated Proteins (GRP94 and GRP78), Rat Growth Hormone, Human Serum Amyloid A (SAA), Troponin I (TN I), Platelet-Derived Growth Factor (PDGF), Duchenne Muscular Dystrophy, SV40, Retroviruses, Papilloma Virus, Hepatitis B Virus, Human Immunodeficiency Virus, Cytomegalovirus (CMV) IE, Gibbon Ape Leukemia Virus.

Inducible elements include, but are not limited to MT II—Phorbol Ester (TFA)/Heavy metals; MMTV (mouse mammary tumor virus)—Glucocorticoids; Interferon—poly(rI)x/poly(rc); Adenovirus 5 E2—E1A; Collagenase—Phorbol Ester (TPA); Stromelysin—Phorbol Ester (TPA); SV40—Phorbol Ester (TPA); Murine MX Gene—Interferon, Newcastle Disease Virus; GRP78 Gene—A23187; 2-Macroglobulin—IL-6; Vimentin—Serum; MHC Class I Gene H-2b—Interferon; HSP70—ElA/SV40 Large T Antigen; Proliferin—Phorbol Ester/TPA; Tumor Necrosis Factor—PMA.

The particular promoter that is employed to control the expression of peptide or protein encoding polynucleotide of the disclosure is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

In embodiments in which a vector is administered to a subject for expression of the protein, it is contemplated that a desirable promoter for use with the vector is one that is not down-regulated by cytokines or one that is strong enough that even if down-regulated, it produces an effective amount of a saeRS-regulated protein for eliciting an immune response. Non-limiting examples of these are CMV IE and RSV LTR. Tissue specific promoters can be used, particularly if expression is in cells in which expression of an antigen is desirable, such as dendritic cells or macrophages. The mammalian MHC I and MHC II promoters are examples of such tissue-specific promoters.

F. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments, the use of internal ribosome entry sites (IRES) elements are used to create multigene, or polycistronic, messages. IRES elements are able to bypass the ribosome scanning model of 5′methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

G. Selectable and Screenable Markers

In certain embodiments, cells containing a nucleic acid construct of the disclosure may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

H. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

I. Expression Systems

Numerous expression systems exist that comprise at least a part or all of the compositions discussed above. Prokaryote- and/or eukaryote-based systems can be employed for use to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems, other examples of expression systems include STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### IX. CHIMERIC ANTIGEN RECEPTORS (CARS)

Aspects of the disclosure relate to novel CAR molecules in which the scFv in the traditional CAR molecule is replaced or supplemented with a modified Fab or protein G Fab binding domain of the disclosure. The embodiments below relate to embodiments that may be included in the polypeptides of the disclosure.

A. Signal Peptide

Polypeptides of the present disclosure may comprise a signal peptide. A “signal peptide” refers to a peptide sequence that directs the transport and localization of the protein within a cell, e.g. to a certain cell organelle (such as the endoplasmic reticulum) and/or the cell surface. In some embodiments, a signal peptide directs the nascent protein into the endoplasmic reticulum. This is essential if a receptor is to be glycosylated and anchored in the cell membrane. Generally, the signal peptide natively attached to the amino-terminal most component is used (e.g. in an scFv with orientation light chain-linker-heavy chain, the native signal of the light-chain is used).

In some embodiments, the signal peptide is cleaved after passage of the endoplasmic reticulum (ER), i.e. is a cleavable signal peptide. In some embodiments, a restriction site is at the carboxy end of the signal peptide to facilitate cleavage.

B. Extracellular Spacer

An extracellular spacer may link an antigen-binding domain, a protein G Fab binding domain, or a Fab to a transmembrane domain. In some embodiments, a hinge is flexible enough to allow the antigen-binding domain to orient in different directions to facilitate antigen binding. In one embodiment, the spacer is the hinge region from IgG. Alternatives include the CH2CH3 region of immunoglobulin and portions of CD3. In some embodiments, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some embodiments, the spacer is from IgG4. An extracellular spacer may comprise a hinge region.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some embodiments, the extracellular spacer consists of or comprises a hinge region from an immunoglobulin (e.g. IgG). Immunoglobulin hinge region amino acid sequences are known in the art; see, e.g., Tan et al. (1990) Proc. Natl. Acad. Sci. USA 87: 162; and Huck et al. (1986) Nucl. Acids Res.

The length of an extracellular spacer may have effects on the CAR's signaling activity and/or the CAR-T cells' expansion properties in response to antigen-stimulated CAR signaling. In some embodiments, a shorter spacer such as less than 50, 45, 40, 30, 35, 30, 25, 20, 15, 14, 13, 12, 11, or 10 amino acids is used. In some embodiments, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids (or any derivable range therein) may have the advantage of increased expansion in vivo or in vitro.

As non-limiting examples, an immunoglobulin hinge region can include one of the following amino acid sequences: DKTHT (SEQ ID NO:125); CPPC (SEQ ID NO:126); CPEPKSCDTPPPCPR (SEQ ID NO:127); ELKTPLGDTTHT (SEQ ID NO:128); KSCDKTHTCP (SEQ ID NO:129); KCCVDCP (SEQ ID NO:130); KYGPPCP (SEQ ID NO:131); EPKSCDKTHTCPPCP (SEQ ID NO:132—human IgG1 hinge); ERKCCVECPPCP (SEQ ID NO:133—human IgG2 hinge); ELKTPLGDTTHTCPRCP (SEQ ID NO:134—human IgG3 hinge); SPNMVPHAHHAQ (SEQ ID NO:135); ESKYGPPCPPCP (SEQ ID NO:136) or ESKYGPPCPSCP (SEQ ID NO:137) (human IgG4 hinge-based) and the like.

The extracellular spacer can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO:138), or a variant thereof.

The extracellular spacer may comprise or further comprise a CH2 region. An exemplary CH2 region is APEFEGGPSVFLFPPKPKDTLMISRTPEVTCVVVDVSQEDPEVQFNWYVDGVEVHNA KTKPREEQFQSTYRVVSVLTVLHQDWLNGKEYKCKVSNKGLPSSIEKTISKAK (SEQ ID NO:139). The extracellular spacer may comprise or further comprise a CH3 region. An exemplary CH3 region is GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO:140).

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some embodiments, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

C. Transmembrane Domain

Polypeptides of the present disclosure may comprise a transmembrane domain. In some embodiments, a transmembrane domain is a hydrophobic alpha helix that spans the membrane. Different transmembrane domains may result in different receptor stability.

In some embodiments, the transmembrane domain is interposed between the extracellular spacer and the cytoplasmic region. In some embodiments, the transmembrane domain is interposed between the extracellular spacer and one or more costimulatory regions. In some embodiments, a linker is between the transmembrane domain and the one or more costimulatory regions.

Any transmembrane domain that provides for insertion of a polypeptide into the cell membrane of a eukaryotic (e.g., mammalian) cell may be suitable for use. As one non-limiting example, the transmembrane sequence FWVLVVVGGVLACYSLLVTVAFIIFWV (SEQ ID NO:141), which is CD28-derived, can be used. In some embodiments, the transmembrane domain is CD8 beta derived: LGLLVAGVLVLLVSLGVAIHLCC (SEQ ID NO:142); CD4 derived: ALIVLGGVAGLLLFIGLGIFFCVRC (SEQ ID NO:143); CD3 zeta derived: LCYLLDGILFIYGVILTALFLRV (SEQ ID NO:144); CD28 derived: WVLVVVGGVLACYSLLVTVAFIIFWV (SEQ ID NO:145); CD134 (OX40) derived: VAAILGLGLVLGLLGPLAILLALYLL (SEQ ID NO:146); or CD7 derived: ALPAALAVISFLLGLGLGVACVLA (SEQ ID NO:147). In some embodiments, the transmembrane domain is derived from CD28, CD8, CD4, CD3-zeta, CD134, or CD7.

D. Cytoplasmic Region

After antigen recognition, receptors of the present disclosure may cluster and a signal transmitted to the cell through the cytoplasmic region. In some embodiments, the costimulatory domains described herein are part of the cytoplasmic region. In some embodiments, the cytoplasmic region comprises an intracellular signaling domain. An intracellular signaling domain may comprise a primary signaling domain and one or more costimulatory domains.

Cytoplasmic regions and/or costimulatiory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the cytoplasmic region includes at least one (e.g., one, two, three, four, five, six, etc.) ITAM motif as described herein. In some embodiments, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

In some cases, the cytoplasmic region is derived from DAP12 (also known as TYROBP; TYRO protein tyrosine kinase binding protein; KARAP; PLOSL; DN AX-activation protein 12; KAR-associated protein; TYRO protein tyrosine kinase-binding protein; killer activating receptor associated protein; killer-activating receptor-associated protein; etc.). For example, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 148)
MGGLEPCSRLLLLPLLLAVSGLRPVQAQAQSDCSCSTVSPGVLAGIVMGD
LVLTVLIALAVYFLGRLVPRGRGAAEAATRKQRITETESPYQELQGQRSD
VYSDLNTQRPYYK;
(SEQ ID NO: 149)
MGGLEPCSRLLLLPLLLAVSGLRPVQAQAQSDCSCSTVSPGVLAGIVMGD
LVLTVLIALAVYFLGRLVPRGRGAAEATRKQRITETESPYQELQGQRSDV
YSDLNTQRPYYK;
(SEQ ID NO: 150)
MGGLEPCSRLLLLPLLLAVSDCSCSTVSPGVLAGIVMGDLVLTVLIALAV
YFLGRLVPRGRGAAEAATRKQRITETESPYQELQGQRSDVYSDLNTQRPY
YK;
or
(SEQ ID NO: 151)
MGGLEPCSRLLLLPLLLAVSDCSCSTVSPGVLAGIVMGDLVLTVLIALAV
YFLGRLVPRGRGAAEATRKQRITETESPYQELQGQRSDVYSDLNTQRPYY
K.

In some embodiments, a suitable cytoplasmic region can comprise an ITAM motif-containing portion of the full length DAP12 amino acid sequence. Thus, a suitable endodomain polypeptide can comprise an amino acid sequence having at least 75%, at least 80%, at least 85%, at least 90%, at least 95%, at least 98%, or 100%, amino acid sequence identity to ESPYQELQGQRSDVYSDLNTQ (SEQ ID NO:152).

In some embodiments, the cytoplasmic region is derived from FCER1G (also known as FCRG; Fc epsilon receptor I gamma chain; Fc receptor gamma-chain; fc-epsilon R1-gamma; fcRgamma; fceRI gamma; high affinity immunoglobulin epsilon receptor subunit gamma; immunoglobulin E receptor, high affinity, gamma chain; etc.). For example, a suitable endodomain polypeptide can comprise an amino acid sequence having at least 75%, at least 80%, at least 85%, at least 90%, at least 95%, at least 98%, or 100% amino acid sequence identity to

(SEQ ID NO: 153)
MIPAVVLLLLLLVEQAAALGEPQLCYILDAILFLYGIVLTLLYCRLKIQV
RKAAITSYEKSDGVYTGLSTRNQETYETLKHEKPPQ.

In some embodiments, a suitable cytoplasmic region can comprise an ITAM motif-containing portion of the full length FCER1G amino acid sequence. Thus, a suitable endodomain polypeptide can comprise an amino acid sequence having at least 75%, at least 80%, at least 85%, at least 90%, at least 95%, at least 98%, or 100%, amino acid sequence identity to DGVYTGLSTRNQETYETLKHE (SEQ ID NO:154).

In some embodiments, the cytoplasmic region is derived from T cell surface glycoprotein CD3 delta chain (also known as CD3D; CD3-DELTA; T3D; CD3 antigen, delta subunit; CD3 delta; CD36; CD3d antigen, delta polypeptide (TiT3 complex); OKT3, delta chain; T cell receptor T3 delta chain; T cell surface glycoprotein CD3 delta chain; etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 170 aa, of either of the following amino acid sequences (2 isoforms):

(SEQ ID NO: 155)
MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQVYQ
PLRDRDDAQYSHLGGNWARNK
or
(SEQ ID NO: 156)
MEHSTFLSGLVLATLLSQVSPFKIPIEELEDRVFVNCNTSITWVEGTVGT
LLSDITRLDLGKRILDPRGIYRCNGTDIYKDKESTVQVHYRTADTQALLR
NDQVYQPLRDRDDAQYSHLGGNWARNK.

In some embodiments, a suitable cytoplasmic region can comprise an ITAM motif-containing portion of the full length CD3 delta amino acid sequence. Thus, a suitable endodomain polypeptide can comprise an amino acid sequence having at least 75%, at least 80%, at least 85%, at least 90%, at least 95%, at least 98%, or 100%, amino acid sequence identity to DQVYQPLRDRDDAQYSHLGGN (SEQ ID NO:157).

In some embodiments, the cytoplasmic region is derived from T cell surface glycoprotein CD3 epsilon chain (also known as CD3e, CD3R; T cell surface antigen T3/Leu-4 epsilon chain, T cell surface glycoprotein CD3 epsilon chain, AI504783, CD3, CD3epsilon, T3e, etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 205 aa, of the following amino acid sequence:

(SEQ ID NO: 158)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI.

In some embodiments, a suitable cytoplasmic region can comprise an ITAM motif-containing portion of the full length CD3 epsilon amino acid sequence. Thus, a suitable endodomain polypeptide can comprise an amino acid sequence having at least 75%, at least 80%, at least 85%, at least 90%, at least 95%, at least 98%, or 100%, amino acid sequence identity to NPDYEPIRKGQRDLYSGLNQR (SEQ ID NO:159).

In some embodiments, the cytoplasmic region is derived from T cell surface glycoprotein CD3 gamma chain (also known as CD3G, CD3γ, T cell receptor T3 gamma chain, CD3-GAMMA, T3G, gamma polypeptide (TiT3 complex), etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 180 aa, of the following amino acid sequence:

(SEQ ID NO: 160)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN.

In some embodiments, a suitable cytoplasmic region can comprise an ITAM motif-containing portion of the full length CD3 gamma amino acid sequence. Thus, a suitable cytoplasmic region can comprise an amino acid sequence having at least 75%, at least 80%, at least 85%, at least 90%, at least 95%, at least 98%, or 100%, amino acid sequence identity to DQLYQPLKDREDDQYSHLQGN (SEQ ID NO:161).

In some embodiments, the cytoplasmic region is derived from T cell surface glycoprotein CD3 zeta chain (also known as CD3Z, CD3C, T cell receptor T3 zeta chain, CD247, CD3-ZETA, CD3H, CD3Q, T3Z, TCRZ, etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences (2 isoforms): MKWKALFTAAILQAQLPITEAQSFGLLDPKLCYLLDGILFIYGVILTALFLRVKFSRSA DAPAYQQGQNQLYNELNLGRREEYDVLDKRRGRDPEMGGKPRRKNPQEGLYNELQ KDKMAEAYSEIGMKGERRRGKGHDGLYQGLSTATKDTYDALHMQALPPR (SEQ ID NO:162) or MKWKALFTAAILQAQLPITEAQSFGLLDPKLCYLLDGILFIYGVILTALFLRVKFSRSA DAPAYQQGQNQLYNELNLGRREEYDVLDKRRGRDPEMGGKPQRRKNPQEGLYNEL QKDKMAEAYSEIGMKGERRRGKGHDGLYQGLSTATKDTYDALHMQALPPR (SEQ ID NO:163). In some embodiments, the cytoplasmic region comprises RVKFSRSADAPAYQQGQNQLYNELNLGRREEYDVLDKRRGRDPEMGGKPRRKNPQ EGLYNELQKDKMAEAYSEIGMKGERRRGKGHDGLYQGLSTATKDTYDALHMQALP PR (SEQ ID NO:164).

In some embodiments, a suitable cytoplasmic region can comprise an ITAM motif-containing portion of the full length CD3 zeta amino acid sequence. Thus, a suitable cytoplasmic region can comprise an amino acid sequence having at least 75%, at least 80%, at least 85%, at least 90%, at least 95%, at least 98%, or 100%, amino acid sequence identity to any of the following amino acid sequences:

(SEQ ID NO: 165)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR;
(SEQ ID NO: 166)
NQLYNELNLGRREEYDVLDKR;
(SEQ ID NO: 167)
EGLYNELQKDKMAEAYSEIGMK;
or
(SEQ ID NO: 168)
DGLYQGLSTATKDTYDALHMQ.

E. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

A costimulatory region may have a length of at least, at most, or exactly 20, 25, 30, 35, 40, 50, 60, 70, 80, 90, 100, 150, 200, or 300 amino acids or any range derivable therein. In some embodiments, the costimulatory region is derived from an intracellular portion of the transmembrane protein 4-1BB (also known as TNFRSF9; CD137; CDwl37; ILA; etc.). For example, a suitable costimulatory region can comprise an amino acid sequence having at least 75%, at least 80%, at least 85%, at least 90%, at least 95%, at least 98%, or 100% amino acid sequence identity to KRGRKKLLYIFKQPFMRPVQTTQEEDGCSCRFPEEEEGGCEL (SEQ ID NO:169).

In some embodiments, the costimulatory region is derived from an intracellular portion of the transmembrane protein CD28 (also known as Tp44). For example, a suitable costimulatory region can comprise an amino acid sequence having at least 75%, at least 80%, at least 85%, at least 90%, at least 95%, at least 98%, or 100% amino acid sequence identity to FWVRSKRSRLLHSDYMNMTPRRPGPTRKHYQPYAPPRDFAAYRS (SEQ ID NO:170).

In some embodiments, the costimulatory region is derived from an intracellular portion of the transmembrane protein ICOS (also known as AILIM, CD278, and CVID1). For example, a suitable costimulatory region can comprise an amino acid sequence having at least 75%, at least 80%, at least 85%, at least 90%, at least 95%, at least 98%, or 100% amino acid sequence identity to TKKKYSSSVHDPNGEYMFMRAVNTAKKSRLTDVTL (SEQ ID NO:171).

In some embodiments, the costimulatory region is derived from an intracellular portion of the transmembrane protein OX-40 (also known as TNFRSF4, RP5-902P8.3, ACT35, CD134, OX40, TXGP1L). For example, a suitable co-stimulatory region can comprise an amino acid sequence having at least 75%, at least 80%, at least 85%, at least 90%, at least 95%, at least 98%, or 100% amino acid sequence identity to RRDQRLPPDAHKPPGGGSFRTPIQEEQADAHSTLAKI (SEQ ID NO:172).

Other exemplary co-stimulatory regions may be derived from an intracellular portion of the transmembrane protein BTLA (also known as BTLA1 and CD272), an intracellular portion of the transmembrane protein CD27 (also known as S 152, T14, TNFRSF7, and Tp55), an intracellular portion of the transmembrane protein CD30 (also known as TNFRSF8, D1S166E, and Ki-1), an intracellular portion of the transmembrane protein GITR (also known as TNFRSF18, RP5-902P8.2, AITR, CD357, and GITR-D), and/or an intracellular portion of the transmembrane protein HVEM (also known as TNFRSF14, RP3-395M20.6, ATAR, CD270, HVEA, HVEM, LIGHTR, and TR2),

### X. DETECTION PEPTIDES

In some embodiments, the polypeptides described herein may further comprise a detection peptide. Suitable detection peptides include hemagglutinin (HA; e.g., YPYDVPDYA (SEQ ID NO:173); FLAG (e.g., DYKDDDDK (SEQ ID NO:174); c-myc (e.g., EQKLISEEDL; SEQ ID NO:175), and the like. Other suitable detection peptides are known in the art.

### XI. LINKERS

In some embodiments, the polypeptides of the disclosure include linkers. In some embodiments, polypeptides of the disclosure are conjugated to other molecules, such as other polypeptides, therapeutic agents, accessory proteins, etc. through a linker. The linker may be a chemical linker or a peptide linker. Thus, embodiments relate to polypeptides conjugated to other molecules through a peptide bond and polypeptides conjugated to other molecules through chemical conjugation.

A peptide linker may be used to separate any of the domain/regions described herein. As an example, a linker may be between the signal peptide and the antigen binding domain, the Fab heavy or light chain region, the protein G Fab binding domain, between the VH and VL of an antigen binding domain, between an antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids (or any derivable range therein).

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G)n, glycine-serine polymers (including, for example, (GS)n, (GSGGS)n (SEQ ID NO:176), (G4S)n (SEQ ID NO: 264) and (GGGS)n (SEQ ID NO:177), where n is an integer of at least one. In some embodiments, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:178), GGSGG (SEQ ID NO:179), GSGSG (SEQ ID NO:180), GSGGG (SEQ ID NO:181), GGGSG (SEQ ID NO:182), GSSSG (SEQ ID NO:183), and the like. Other near neutral amino acids, such as Thr and Ala, may also be used in the linker sequence. The length of the linker sequence may vary without significantly affecting the function or activity of the fusion protein (see, e.g., U.S. Pat. No. 6,087,329). In a particular aspect, the linker may be at least, at most, or exactly 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acid residues (or any range derivable therein).

Examples of linkers may also include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST). Examples of linkers further comprise a linear carbon chain, such as C_{N} (where N=1-100 carbon atoms). In some embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (vc) linker. In some embodiments, the linker is sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its sulfo-NHS ester is reactive toward primary amines (as found in lysine and the protein or peptide N-terminus). Further, the linker may be maleimidocaproyl (mc). In some embodiments, the covalent linkage may be achieved through the use of Traut's reagent.

### XII. CELLS

Certain embodiments relate to cells comprising polypeptides or nucleic acids of the disclosure. In some embodiments the cell is an immune cell or a T cell. “T cell” includes all types of immune cells expressing CD3 including T-helper cells, invariant natural killer T (iNKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### XIII. METHODS FOR MODIFYING GENOMIC DNA

In certain embodiments, the genomic DNA is modified either to include additional mutations, insertions, or deletions, or to integrate certain molecular constructs of the disclosure so that the constructs are expressed from the genomic DNA. In some embodiments, a nucleic acid encoding a polypeptide of the disclosure is integrated into the genomic DNA of a cell. In some embodiments, a nucleic acid is integrated into a cell via viral transduction, such as gene transfer by lentiviral or retroviral transduction. In some embodiments, genomic DNA is modified by integration of nucleic acid encoding a polypeptide of the present disclosure (e.g., a CAR) into the genome of a host cell via a retroviral vector, a lentiviral vector, or an adeno-associated viral vector.

In some embodiments, the integration is targeted integration. In some embodiments, targeted integration is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids. One specific target is the TRAC (T cell receptor alpha constant) locus. For instance, cells would first be electroporated with a ribonucleoprotein (RNP) complex consisting of Cas9 protein complexed with a single-guide RNA (sgRNA) targeting the TRAC (T cell receptor alpha constant) locus. Fifteen minutes post electroporation, the cells would be treated with AAV6 carrying the HDR template that encodes for the CAR. In another example, double stranded or single stranded DNA comprises the HDR template and is introduced into the cell via electroporation together with the RNP complex.

Therefore, one aspect, the current disclosure includes targeted integration. One way of achieving this is through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, (DC31 integrase, Bxb1-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRISPR/Cas-like endonucleases, I-Tevl nucleases or related monomeric hybrids, or artificial targeted DNA double strand break inducing agents. Exemplary targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named “LAGLIDADG” (SEQ ID NO: 265) has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31(11):2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7(1):171-192; Bogdanove A J, Voytas D F., 2011, Science, 333(6051):1843-6; Bradley P, Bogdanove A J, Stoddard B L., 2013, Curr Opin Struct Biol., 23(1):93-9.

Another exemplary targeting endonuclease is a site-specific nuclease. In particular, the site-specific nuclease may be a “rare-cutter” endonuclease whose recognition sequence occurs rarely in a genome. Preferably, the recognition sequence of the site-specific nuclease occurs only once in a genome. Alternatively, the targeting nuclease may be an artificial targeted DNA double strand break inducing agent.

In some embodiments, targeted integrated can be achieved through the use of an integrase. For example, The phiC31 integrase is a sequence-specific recombinase encoded within the genome of the bacteriophage phiC31. The phiC31 integrase mediates recombination between two 34 base pair sequences termed attachment sites (att), one found in the phage and the other in the bacterial host. This serine integrase has been show to function efficiently in many different cell types including mammalian cells. In the presence of phiC31 integrase, an attB-containing donor plasmid can be unidirectional integrated into a target genome through recombination at sites with sequence similarity to the native attP site (termed pseudo-attP sites). phiC31 integrase can integrate a plasmid of any size, as a single copy, and requires no cofactors. The integrated transgenes are stably expressed and heritable.

In one embodiment, genomic integration of polynucleotides of the disclosure is achieved through the use of a transposase. For example, a synthetic DNA transposon (e.g. “Sleeping Beauty” transposon system) designed to introduce precisely defined DNA sequences into the chromosome of vertebrate animals can be used. The Sleeping Beauty transposon system is composed of a Sleeping Beauty (SB) transposase and a transposon that was designed to insert specific sequences of DNA into genomes of vertebrate animals. DNA transposons translocate from one DNA site to another in a simple, cut-and-paste manner. Transposition is a precise process in which a defined DNA segment is excised from one DNA molecule and moved to another site in the same or different DNA molecule or genome.

As do all other Tc1/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, in which case the transposon becomes a non-autonomous element. Non-autonomous transposons are most useful as genetic tools because after insertion they cannot independently continue to excise and re-insert. All of the DNA transposons identified in the human genome and other mammalian genomes are non-autonomous because even though they contain transposase genes, the genes are non-functional and unable to generate a transposase that can mobilize the transposon.

### XIV. METHODS OF TREATING DISEASES

The compositions of the disclosure may be used for in vivo, in vitro, or ex vivo administration. The route of administration of the composition may be, for example, intracutaneous, subcutaneous, intravenous, local, topical, and intraperitoneal administrations. The compositions and methods of the disclosure may be used to treat an autoimmune disease, a bacterial infection, cancer, or a viral infection, for example.

The autoimmune condition or inflammatory condition amenable for treatment may include, but not be limited to conditions such as diabetes (e.g. type 1 diabetes), graft rejection, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and systemic juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), and adult onset diabetes mellitus (Type II diabetes) and autoimmune diabetes. Also contemplated are immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and gianT cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), Addison's disease, autoimmune neutropenia, pancytopenia, leukopenia, diseases involving leukocyte diapedesis, CNS inflammatory disorders, Alzheimer's disease, Parkinson's disease, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, thermal injury, preeclampsia, an immune complex disorder such as immune complex nephritis, antibody-mediated nephritis, polyneuropathies, chronic neuropathy such as IgM polyneuropathies or IgM-mediated neuropathy, autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), experimental autoimmune encephalomyelitis, myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, gianT cell hepatitis, chronic active hepatitis or autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia greata, alopecia totalis, CREST syndrome (calcinosis, Raynaud's phenomenon, esophageal dysmotility, sclerodactyl), and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, parasitic diseases such as leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis (acute or chronic), or Fuch's cyclitis, Henoch-Schonlein purpura, human immunodeficiency virus (HIV) infection, SCID, acquired immune deficiency syndrome (AIDS), echovirus infection, sepsis, endotoxemia, pancreatitis, thyroxicosis, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, gianT cell polymyalgia, chronic hypersensitivity pneumonitis, keratoconjunctivitis sicca, epidemic keratoconjunctivitis, idiopathic nephritic syndrome, minimal change nephropathy, benign familial and ischemia-reperfusion injury, transplant organ reperfusion, retinal autoimmunity, joint inflammation, bronchitis, chronic obstructive airway/pulmonary disease, silicosis, aphthae, aphthous stomatitis, arteriosclerotic disorders, asperniogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, non-malignant thymoma, vitiligo, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, autoimmune polyglandular syndrome type I, adult-onset idiopathic hypoparathyroidism (AOIH), cardiomyopathy such as dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia syndrome, angiectasis, autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization, allergic hypersensitivity disorders, glomerulonephritides, reperfusion injury, ischemic re-perfusion disorder, reperfusion injury of myocardial or other tissues, lymphomatous tracheobronchitis, inflammatory dermatoses, dermatoses with acute inflammatory components, multiple organ failure, bullous diseases, renal cortical necrosis, acute purulent meningitis or other central nervous system inflammatory disorders, ocular and orbital inflammatory disorders, granulocyte transfusion-associated syndromes, cytokine-induced toxicity, narcolepsy, acute serious inflammation, chronic intractable inflammation, pyelitis, endarterial hyperplasia, peptic ulcer, valvulitis, graft versus host disease, contact hypersensitivity, asthmatic airway hyperreaction, and endometriosis.

The cancers amenable for treatment include, but are not limited to, tumors of all types, locations, sizes, and characteristics. In some embodiments, the cancer comprises a solid tumor. In some embodiments, the methods relate to reducing tumor volume or treating cancers that are recurrent and/or metastatic. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### XV. ADDITIONAL THERAPIES

A. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

1. Checkpoint Inhibitors and Combination Treatment

Embodiments of the disclosure may include administration of immune checkpoint inhibitors (also referred to as checkpoint inhibitor therapy), which are further described below. The checkpoint inhibitor therapy may be a monotherapy, targeting only one cellular checkpoint proteins or may be combination therapy that targets at least two cellular checkpoint proteins. For example, the checkpoint inhibitor monotherapy may comprise one of: a PD-1, PD-L1, or PD-L2 inhibitor or may comprise one of a CTLA-4, B7-1, or B7-2 inhibitor. The checkpoint inhibitor combination therapy may comprise one of: a PD-1, PD-L1, or PD-L2 inhibitor and, in combination, may further comprise one of a CTLA-4, B7-1, or B7-2 inhibitor. The combination of inhibitors in combination therapy need not be in the same composition, but can be administered either at the same time, at substantially the same time, or in a dosing regimen that includes periodic administration of both of the inhibitors, wherein the period may be a time period described herein.

### a. PD-1, PD-L 1, and PD-L2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect, the PD-1 ligand binding partners are PD-L1 and/or PD-L2. In another embodiment, a PD-L1 inhibitor is a molecule that inhibits the binding of PD-L1 to its binding partners. In a specific aspect, PD-L1 binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-L1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDIO680, also known as AMP-514, and REGN2810.

In some embodiments, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PD-L1, or PD-L2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### b. CTLA-4, B7-1, and B7-2 Inhibitors

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment, they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

5. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Tumor targeted T cells can be generated through gene therapy. Tumor targeted T cells can be expanded by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

B. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

C. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

D. Neoantigens

In some embodiments, the additional therapy comprises targeting of neoantigen mutations. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

E. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

F. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### XVI. PHARMACEUTICAL COMPOSITIONS

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., compositions comprising cells expressing a therapeutic receptor).

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### XVII. SEQUENCES

SEQ ID NO:20 corresponds to the wild-type protein G from Streptococcus or a portion of the wild-type protein G from Streptococcus.

(SEQ ID NO: 20)
EFNKYGVSDYYKNLINNAKTVEGVKDLQAQVVESAKKARISEATDGLSDF
LKSQTPAEDTVKSIELAEAKVLANRELDKYGVSDYHKNLINNAKTVEGVK
DLQAQVVESAKKARISEATDGLSDFLKSQTPAEDTVKSIELAEAKVLANR
ELDKYGVSDYYKNLINNAKTVEGVKALIDEILAALPKTDTYKLILNGKTL
KGETTTEAVDAATAEKVFKQYANDNGVDGEWTYDDATKTFTVTEKPEVID
ASELTPAVTTYKLVINGKTLKGETTTEAVDAATAEKVFKQYANDNGVDGE
WTYDDATKTFTVTEKPEVIDASELTPAVTTYKLVINGKTLKGETTTKAVD
AETAEKAFKQYANDNGVDGVWTYDDATKTFTVTEMVTEVPGDAPTEPEKP
EASIPLVPLTPATPIAKDDAKKDDTKKEDAKKPEAKKEDAKKAETLPTTG
EGSNPFFTAAALAVMAGAGALAVASKRKED

SEQ ID NO:2 refers to a modified protein G Fab binding domain: X₁₅TX₁₇X₁₈X₁₉X₂₀X₂₁X₂₂TX₂₄X_{A}X₃₇Z; wherein X₁₅ is K, R, E, or I; X₁₇ is L, F, or A; X₁₈ is K, S, W, R, or T; X₁₉ is G or Y; X₂₀ is E, Y, A, or H; X₂₁ is T or R; X₂₂ is T, S, A, or G; X₂₄ is E, K, T, or Q; X₃₇ is Q or R; Z comprises an isotype recognition region; and X_{A} is an amino acid sequence that is 5 to 20 amino acids in length.

In some embodiments, the modified protein G Fab binding domain comprises one of SEQ ID NO:3-5, 31-37, or 256: KTLKGETTTKAVDAATAEKVFKQYANDNG (WT—SEQ ID NO:23) RTLSGYTTTTAVDAATAEKVFKQYAYVHE (A1—SEQ ID NO:3); RTLSGYTTTTAVDAATAEKVFKQYAFGNG (F—SEQ ID NO:4); RTLSGYTTTTAVDAATAEKVFKQIDMVSS (D—SEQ ID NO:5); KTFWGETTTKAVDAATAEKVFKQYAFDND (A3—SEQ ID NO:31); ETLRYETSTKAVDAATAEKVFKQIAHDQG (A12—SEQ ID NO:32); KTLKGETTTKAVDAATAEKVFKQYAYVHD (B9—SEQ ID NO:33); ETLRYETSTKAVDAATAEKVFKRIAHDQG (F5—SEQ ID NO:34); KTASGARATKAVDAATAEKVFKQYAKEYP (G11—SEQ ID NO:35); ETLTGETGTQAVDAATAEKVFKQYAWVND (H11—SEQ ID NO:36); ITLKGHTTTKAVDAATAEKVFKQYAWVND (H12—SEQ ID NO:37); and RTLSGYTTTTAVDAATAEEVFKQYAYVHE (A1 K32E—SEQ ID NO:256).

Exemplary linkers disclosed herein include: GGGS (SEQ ID NO:38); GGGSGGGSGGGS (SEQ ID NO:39); LAAA (SEQ ID NO:40); LGGGGSGGGGSGGGGSAAA (SEQ ID NO:41) or LSGGGGSGGGGSGGGGSGGGGSAAA (SEQ ID NO:42); a helical linker such as LAEAAAKEAAAKAAA SEQ ID NO:43), LAEAAAKEAAAKEAAAKAAA (SEQ ID NO:44), LAEAAAKEAAAKEAAAKEAAAKAAA (SEQ ID NO:45), or LAEAAAKEAAAKEAAAKEAAAKEAAAKAAA (SEQ ID NO:46).

In some embodiments, the polypeptides of the disclosure comprise or further comprise an immunogenicity region. In some embodiments, the immunogenicity region comprises KLVINGRTLSG (SEQ ID NO:47)

In some embodiments, the isotype recognition region comprises a region corresponding to a.a. 162-167 of SEQ ID NO:23: YANDNG (SEQ ID NO:48)

Substitute isotype recognition regions include: YAYVHE (Protein-G-HS A1, SEQ ID NO:49); YSRPHV (Protein-G-HS C6, SEQ ID NO:50); YAVGAV (Protein-G-HS C7, SEQ ID NO:51); YAAPHV (Protein-G-HS D2, SEQ ID NO: 52); YSHPHV (Protein-G-HS E3, SEQ ID NO:53); CTVWPV (Protein-G-HS F1, SEQ ID NO:54); YAFAHV (Protein-G-HS H10, SEQ ID NO:55); YAFGNG (SEQ ID NO:10), and IDMVSS (SEQ ID NO:11).

WT immunogenicity region includes: LVINGRTLSG (WT, SEQ ID NO:57); variant immunogenicity regions include: LVIRGLTLSL (B11, SEQ ID NO:58); LVIRGLTLSF (B12, SEQ ID NO:59); LVIGGLRLWF (B5, SEQ ID NO:60); LVIRGVTLLF (B6, SEQ ID NO:61); LVIRGITLGF (B7, SEQ ID NO:62); LVIMGSTLSL (B8, SEQ ID NO:63); LVIIGRTLSL (B9, SEQ ID NO:64); LVISGITLSF (B10, SEQ ID NO:65); LVIGGRTLSF (A11, SEQ ID NO:66); LVIGGRTLSF (A12, SEQ ID NO:67); LVISGSTLSL (B1, SEQ ID NO:68); LVILGRTLSV (B2, SEQ ID NO:69); FVIRGRTLSF (B3, SEQ ID NO:70); LVISGRTLSL (B4, SEQ ID NO:71); LVIGGRTLRF (A8, SEQ ID NO:72); LVIRGVTLGF (A9, SEQ ID NO:73); LVIRGRTLSL (A10, SEQ ID NO:74); LVIGGRTLRF (A1, SEQ ID NO:75); LVIGGRTLSF (A2, SEQ ID NO:76); LVISGLTLSF (A3, SEQ ID NO:77); LVIGGVTLSF (A4, SEQ ID NO:78); LVIRGVTLSL (A5, SEQ ID NO:79); and LVIGGITLSF (A6, SEQ ID NO:80).

In some embodiments, the variant immunogenicity region is at least 80% homologous or identical to X_{2′}VIX_{5′}GX_{7′}X_{8′}LX_{10′}X_{11′} (SEQ ID NO:81), wherein X_{2′} is L or F; X_{5′} is N, R, G, M, I, S, or L; X_{7′} is R, L, V, I, or S; X_{8′} is T or R; X_{10′} is S, W, L, G, or R; X_{11′} is L, F, or V; and wherein the variant immunogenicity region is not LVINGRTLSG (SEQ ID NO:57). In some embodiments, X_{A}=AVDAATAEKVFK (SEQ ID NO:82); X_{B}=LTPAVTTYKLVING (SEQ ID NO:83); X_{C}=VDGEWTYDDATKTFTVTEKPEVI (SEQ ID NO:84). The following sequences comprise exemplary polypeptide embodiments of the disclosure, such as exemplary polypeptide comprising protein G Fab binding domains:

Protein-G-A1:
(SEQ ID NO: 27)
MKGETTTEAVDAATAEKVFKQYANDNGVDGEWTYDDATKTFTVTEKPEVI
DASELTPAVTTYKLVINGRTLSGYTTTTAVDAATAEKVFKQYAYVHEVDG
EWTYDDATKTFTVTEKPEVIDASELTPAVTTYKLVINGRTLSGETTTKAV
DAETAEKAFKQYANDNGVDGVWTYDDATKTFTVTE.
Protein G-F:
(SEQ ID NO: 85)
MKGETTTEAVDAATAEKVFKQYANDNGVDGEWTYDDATKTFTVTEKPEVI
DASELTPAVTTYKLVINGRTLSGYTTTTAVDAATAEKVFKQYAFGNGVDG
EWTYDDATKTFTVTEKPEVIDASELTPAVTTYKLVINGRTLSGETTTKAV
DAETAEKAFKQYANDNGVDGVWTYDDATKTFTVTE.
Protein G-F:
(SEQ ID NO: 86)
MKGETTTEAVDAATAEKVFKQYANDNGVDGEWTYDDATKTFTVTEKPEVI
DASELTPAVTTYKLVINGRTLSGYTTTTAVDAATAEKVFKQIDMVSSVDG
EWTYDDATKTFTVTEKPEVIDASELTPAVTTYKLVINGRTLSGETTTKAV
DAETAEKAFKQYANDNGVDGVWTYDDATKTFTVTE.
Protein-G-A3:
(SEQ ID NO: 87)
MKGETTTEAVDAATAEKVFKQYANDNGVDGEWTYDDATKTFTVTEKPEVI
DASELTPAVTTYKLVINGKTFWGETTTKAVDAATAEKVFKQYAFDNDVDG
EWTYDDATKTFTVTEKPEVIDASELTPAVTTYKLVINGRTLSGETTTKAV
DAETAEKAFKQYANDNGVDGVWTYDDATKTFTVTE
Protein-G-A12:
(SEQ ID NO: 88)
MKGETTTEAVDAATAEKVFKQYANDNGVDGEWTYDDATKTFTVTEKPEVI
DASELTPAVTTYKLVINGETLRYETSTKAVDAATAEKVFKQIAHDQGVDG
EWTYDDATKTFTVTEKPEVIDASELTPAVTTYKLVINGRTLSGETTTKAV
DAETAEKAFKQYANDNGDGVWTYDDATKTFTVTE
Protein-G-B9:
(SEQ ID NO: 89)
MKGETTTEAVDAATAEKVFKQYANDNGVDGEWTYDDATKTFTVTEKPEVI
DASELTPAVTTYKLVINGKTLKGETTTKAVDAATAEKVFKQYAYVHDVDG
EWTYDDATKTFTVTEKPEVIDASELTPAVTTYKLVINGRTLSGETTTKAV
DAETAEKAFKQYANDNGVDGVWTYDDATKTFTVTE
Protein-G-F5:
(SEQ ID NO: 90)
MKGETTTEAVDAATAEKVFKQYANDNGVDGEWTYDDATKTFTVTEKPEVI
DASELTPAVTTYKLVINGETLRYETSTKAVDAATAEKVFKRIAHDQGVDG
EWTYDDATKTFTVTEKPEVIDASELTPAVTTYKLVINGRTLSGETTTKAV
DAETAEKAFKQYANDNGVDGVWTYDDATKTFTVTE
Protein-G-G11:
(SEQ ID NO: 91)
MKGETTTEAVDAATAEKVFKQYANDNGVDGEWTYDDATKTFTVTEKPEVI
DASELTPAVTTYKLVINGKTASGARATKAVDAATAEKVFKQYAKEYPVDG
EWTYDDATKTFTVTEKPEVIDASELTPAVTTYKLVINGRTLSGETTTKAV
DAETAEKAFKQYANDNGVDGVWTYDDATKTFTVTE
Protein-G-H11:
(SEQ ID NO: 92)
MKGETTTEAVDAATAEKVFKQYANDNGVDGEWTYDDATKTFTVTEKPEVI
DASELTPAVTTYKLVINGETLTGETGTQAVDAATAEKVFKQYAWVNDVDG
EWTYDDATKTFTVTEKPEVIDASELTPAVTTYKLVINGRTLSGETTTKAV
DAETAEKAFKQYANDNGVDGVWTYDDATKTFTVTE
Protein-G-H12:
(SEQ ID NO: 93)
MKGETTTEAVDAATAEKVFKQYANDNGVDGEWTYDDATKTFTVTEKPEVI
DASELTPAVTTYKLVINGITLKGHTTTKAVDAATAEKVFKQYAWVNDVDG
EWTYDDATKTFTVTEKPEVIDASELTPAVTTYKLVINGRTLSGETTTKAV
DAETAEKAFKQYANDNGVDGVWTYDDATKTFTVTE

Protein G Sequences that demonstrate isotype specificity.

Protein-G-HS A1:
(SEQ ID NO: 94)
MKGETTTEAVDAATAEKVFKQYANDNGVDGEWTYDDATKTFTVTEKPEVI
DASELTPAVTTYKLVINGKTLKGETTTEAVDAATAEKVFKQYANDNGVDG
EWTYDDATKTFTVTEKPEVIDASELTPAVTTYKLVINGRTLSGETTTKAV
DAETAEKAFKQYAYVHEVDGVWTYDDATKTFTVTE
Protein-G-HS C6:
(SEQ ID NO: 95)
MKGETTTEAVDAATAEKVFKQYANDNGVDGEWTYDDATKTFTVTEKPEVI
DASELTPAVTTYKLVINGKTLKGETTTEAVDAATAEKVFKQYANDNGVDG
EWTYDDATKTFTVTEKPEVIDASELTPAVTTYKLVINGRTLSGETTTKAV
DAETAEKAFKQYSRPHVVDGVWTYDDATKTFTVTE
Protein-G-HS C7:
(SEQ ID NO: 96)
MKGETTTEAVDAATAEKVFKQYANDNGVDGEWTYDDATKTFTVTEKPEVI
DASELTPAVTTYKLVINGKTLKGETTTEAVDAATAEKVFKQYANDNGVDG
EWTYDDATKTFTVTEKPEVIDASELTPAVTTYKLVINGRTLSGETTTKAV
DAETAEKAFKQYAYGAVVDGVWTYDDATKTFTVTE
Protein-G-HS D2:
(SEQ ID NO: 97)
MKGETTTEAVDAATAEKVFKQYANDNGVDGEWTYDDATKTFTVTEKPEVI
DASELTPAVTTYKLVINGKTLKGETTTEAVDAATAEKVFKQYANDNGVDG
EWTYDDATKTFTVTEKPEVIDASELTPAVTTYKLVINGRTLSGETTTKAV
DAETAEKAFKQYAAPHVVDGVWTYDDATKTFTVTE
Protein-G-HS E3:
(SEQ ID NO: 98)
MKGETTTEAVDAATAEKVFKQYANDNGVDGEWTYDDATKTFTVTEKPEVI
DASELTPAVTTYKLVINGKTLKGETTTEAVDAATAEKVFKQYANDNGVDG
EWTYDDATKTFTVTEKPEVIDASELTPAVTTYKLVINGRTLSGETTTKAV
DAETAEKAFKQYSHPHVVDGVWTYDDATKTFTVTE
Protein-G-HS F1:
(SEQ ID NO: 99)
MKGETTTEAVDAATAEKVFKQYANDNGVDGEWTYDDATKTFTVTEKPEVI
DASELTPAVTTYKLVINGKTLKGETTTEAVDAATAEKVFKQYANDNGVDG
EWTYDDATKTFTVTEKPEVIDASELTPAVTTYKLVINGRTLSGETTTKAV
DAETAEKAFKQCTVWPVVDGVWTYDDATKTFTVTE
Protein-G-HS H10:
(SEQ ID NO: 100)
MKGETTTEAVDAATAEKVFKQYANDNGVDGEWTYDDATKTFTVTEKPEVI
DASELTPAVTTYKLVINGKTLKGETTTEAVDAATAEKVFKQYANDNGVDG
EWTYDDATKTFTVTEKPEVIDASELTPAVTTYKLVINGRTLSGETTTKAV
DAETAEKAFKQYAFAHVVDGVWTYDDATKTFTVTE
Immunogenicity
B11:
(SEQ ID NO: 101)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIRGLTLSLETTTKAV
DAETAEKAFKQYANDNGVDGVWTYDDATKTFTVTE
B12:
(SEQ ID NO: 102)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIRGLTLSFETTTKAV
DAETAEKAFKQYANDNGVDGVWTYDDATKTFTVTE
B5:
(SEQ ID NO: 103)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIGGLRLWFETTTKAV
DAETAEKAFKQYANDNGVDGVWTYDDATKTFTVTE
B6:
(SEQ ID NO: 104)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIRGVTLLFETTTKAV
DAETAEKAFKQYANDNGVDGVWTYDDATKTFTVTE
B7:
(SEQ ID NO: 105)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
FEWTYDDATKTTVTEKPEVIDASELTPAVTTYKLVIRGITLGFETTTKAV
DAETAEKAFKQYANDNGVDGVWTYDDATKTFTVTE
B8:
(SEQ ID NO: 106)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIMGSTLSLETTTKAV
DAETAEKAFKQYANDNGVDGVWTYDDATKTFTVTE
B9:
(SEQ ID NO: 107)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIIGRTLSLETTTKAV
DAETAEKAFKQYANDNGVDGVWTYDDATKTFTVTE
B10:
(SEQ ID NO: 108)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVISGITLSFETTTKAV
DAETAEKAFKQYANDNGVDGVWTYDDATKTFTVTE
A11:
(SEQ ID NO: 109)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIGGRTLSFETTTKAV
DAETAEKAFKQYANDNGVDGVWTYDDATKTFTVTE
A12:
(SEQ ID NO: 110)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIGGRTLSFETTTKAV
DAETAEKAFKQYANDNGVDGVWTYDDATKTFTVTE
B1:
(SEQ ID NO: 111)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVISGSTLSLETTTKAV
DAETAEKAFKQYANDNGVDGVWTYDDATKTFTVTE
B2:
(SEQ ID NO: 112)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVILGRTLSVETTTKAV
DAETAEKAFKQYANDNGVDGVWTYDDATKTFTVTE
B3:
(SEQ ID NO: 113)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKFVIRGRTLSFETTTKAV
DAETAEKAFKQYANDNGVDGVWTYDDATKTFTVTE
B4
(SEQ ID NO: 114)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVISGRTLSLETTTKAV
DAETAEKAFKQYANDNGVDGVWTYDDATKTFTVTE
A8:
(SEQ ID NO: 115)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIGGRTLRFETTTKAV
DAETAEKAFKQYANDNGVDGVWTYDDATKTFTVTE
A9:
(SEQ ID NO: 116)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIRGVTLGFETTTKAV
DAETAEKAFKQYANDNGVDGVWTYDDATKTFTVTE
A10:
(SEQ ID NO: 117)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIRGRTLSLETTTKAV
DAETAEKAFKQYANDNGVDGVWTYDDATKTFTVTE
A1:
(SEQ ID NO: 118)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIGGRTLRFETTTKAV
DAETAEKAFKQYANDNGVDGVWTYDDATKTFTVTE
A2:
(SEQ ID NO: 119)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIGGRTLSFETTTKAV
DAETAEKAFKQYANDNGVDGVWTYDDATKTFTVTE
A3:
(SEQ ID NO: 120)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVISGLTLSFETTTKAV
DAETAEKAFKQYANDNGVDGVWTYDDATKTFTVTE
A4:
(SEQ ID NO: 121)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIGGVTLSFETTTKAV
DAETAEKAFKQYANDNGVDGVWTYDDATKTFTVTE
A5:
(SEQ ID NO: 122)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIRGVTLSLETTTKAV
DAETAEKAFKQYANDNGVDGVWTYDDATKTFTVTE
A6:
(SEQ ID NO: 123)
MKGETTTEAVDAATAEKVFKQYANDNGVDGEWTYDDATKTFTVTEKPEVI
DASELTPAVTTYKLVINGKTLKGETTTKAVDAATAEKVFKQYANDNGVDG
EWTYDDATKTFTVTEKPEVIDASELTPAVTTYKLVIGGITLSFETTTKAV
DAETAEKAFKQYANDNGVDGVWTYDDATKTFTVTE.
XVIII. Kits

Certain aspects of the disclosure also concern kits containing compositions of the invention or compositions to implement methods of the invention. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, binding agents, detection agents, or any value or range and combination derivable therein. In some embodiments, there are kits for evaluating antigens in a composition, in tissues, in cells, or in a composition suspected of comprising antigenic or peptidic components.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. In certain aspects, negative and/or positive control nucleic acids, probes, binding agents, and inhibitors are included in some kit embodiments.

Embodiments of the disclosure include kits for analysis of a pathological sample by assessing the presence or absence of one or more peptides, polypeptides, or antigens. The kit can further comprise reagents for detecting or binding labels, tags, and enzymatic reactions. The kit may also include labeling reagents, including at least one of amine-modified nucleotide, poly(A) polymerase, and poly(A) polymerase buffer. Labeling reagents can include an amine-reactive dye.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

### XIX. EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. In the examples and throughout the disclosure, Protein-G variants are polypeptides comprising a Fab binding domain.

### Example 1—an Engineered Ultra-High Affinity Fab-Protein G Pair Enables a Modular Antibody Platform with Multifunctional Capability

Engineered recombinant antibody-based reagents are rapidly supplanting traditionally derived antibodies in many cell biological applications. A particularly powerful aspect of these engineered reagents is that other modules having myriad functions can be attached to them either chemically or through molecular fusions. However, these processes can be cumbersome and do not lend themselves to high throughput applications. Consequently, the inventors have endeavored to develop a platform that can introduce multiple functionalities into a class of Fab-based affinity reagents in a “plug and play” fashion. This platform exploits the ultra-tight binding interaction between affinity matured variants of a Fab scaffold (Fab^{S}) and a domain of an immunoglobulin binding protein, protein G (GA1). GA1 is easily genetically manipulatable facilitating the ability to link these modules together like beads on a string with adjustable spacing to produce multivalent and bi-specific entities. GA1 can also be fused to other proteins or be chemically modified to engage other types of functional components. To demonstrate the utility for the Fab-GA1 platform, the inventors applied it to a detection proximity assay based on the β-lactamase (BL) split enzyme system. The inventors also show the bi-specific capabilities of the module by using it in context of a Bi-specific T-cell engager (BiTE), which is a therapeutic assemblage that induces cell killing by crosslinking T-cells to cancer cells. The inventors show that GA1-Fab modules are easily engineered into potent cell killing BiTE-like assemblages and have the advantage of interchanging Fabs directed against different cell surface cancer related targets in a plug and play fashion.

Affinity reagents are the cornerstone of cell biology. They come in many manifestations, but antibodies are by far the most widely used format. Traditionally, antibodies were produced using animal immunization methodologies (1). While this approach is still in broad use, recombinant display technologies have now assumed the leading role in producing antibody-based affinity reagents (2-4). Recombinant reagents have manifold advantages over traditionally produced monoclonal antibodies; for instance, economic and scalable production and permanent archiving are notable advantages (5, 6). While monoclonal antibodies can be reproduced, the maintenance and large-scale culture of hybridoma cells can be cumbersome and expensive. However, the most compelling advantage of the recombinant approach is that display methods can be used to customize the affinity binders in ways not accessible by monoclonal antibodies (7-9). For instance, selection conditions to produce affinity reagents can be tuned to direct binders to target particular conformation states or bind a specific epitope (10, 11). Thus, the user has much more control over the characteristics of the affinity reagent being produced.

Over the last decade, the inventors have developed a high throughput pipeline for the rapid production of antibody Fab-based affinity reagents using phage display mutagenesis (6, 12). The Fab CDR libraries used are based on the Herceptin Fab scaffold, which has been engineered for stability and expression. The phage display biopanning protocols employed by the inventors allow exquisite control of the properties of the Fabs being selected for and generally multiple high quality binders can be obtained for a given target (6, 8, 13). These binders have been utilized in a number of cell biological and biochemical applications (14-16). Further, they have been exploited as powerful crystallization chaperones and fiducial marks for single-particle cryo-EM structural studies (17-21).

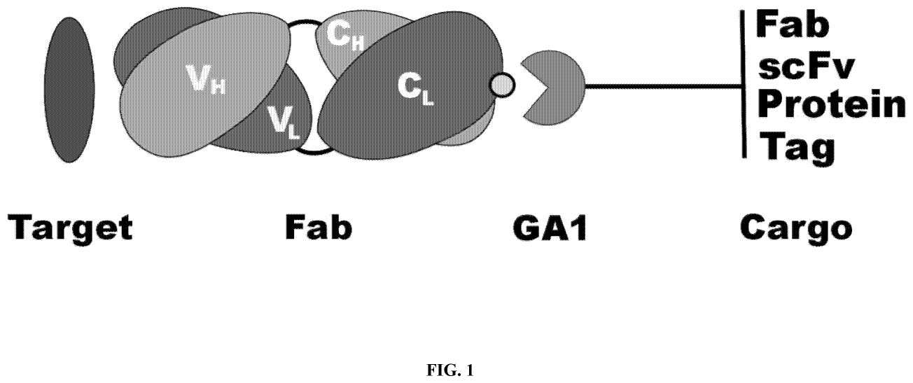

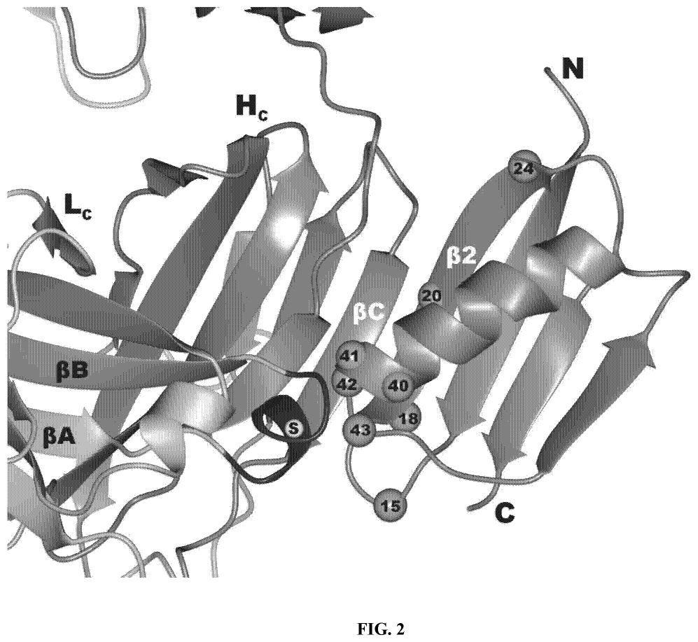

While a principal reason for utilization of Fabs in many standard applications is due to their ease of production, it is their engineerability that makes them candidates for more sophisticated types of applications. In particular, Fabs are stable modules that are easily adapted to fusing or chemically linking other molecular entities to them for imaging and numerous biochemical manipulations (22). One challenge faced by antibody engineers has been to develop user-friendly ways to endow these modules with multivalent or multi-specificity properties. The key is to design simplified systems that can be used by cell biologists or biochemists that do not require significant expertise in protein engineering. A goal would be to combine Fabs as modules like lego blocks, pieces of which could be pre-fabricated as a unit and then combined with other Fabs of other specificities to generate a variety of bi-specific or multivalent entities in a plug and play fashion. Towards this end, the inventors have developed a Fab binding module based on Protein G (PG) that can be fused onto many different molecular components. As such, they can be assembled in a variety of different formats in a straightforward way, allowing the researcher to design highly customized affinity reagents (FIG. 1).

Herein, this example describes a platform that uses engineered Fab-based modules to perform a series of complex tasks outside of the capabilities of traditional antibodies. A key component of the modules is an affinity matured variant (GA1) derived from the immunoglobulin binding domain, Protein G (PG). Importantly, GA1 binds to an epitope on the constant domain of the Fab far removed from its antigen binding loops. The inventors had previously shown that Fabs can bind to GA1 domains that have been linked together to form multivalent entities (23). However, the initial GA1-Fab affinity was ˜50 nM, which the inventors deemed insufficient for the types of functions the inventors envisioned for the modules described here. Therefore, using a stepwise phage display mutagenesis approach, the inventors produced variants of the Fab scaffold (Fab^{LRT}) that form a complex with GA1 endowed with an ultra-high affinity (100 pM). Exploiting the high affinity between Fabs and GA1, modules were designed and tested that facilitate high throughput sandwich assays, proximity complementary assays (PCA) and fabrication of potent bi-specific T-cell engagers (BiTES). The modules are designed to have interchangeable parts allowing a broad range of combinations that can be evaluated in a multiplexed fashion [FIG. 1]. Importantly, the work presents a blueprint to guide other protein engineers for how to expand the system for myriad applications.

A. Results

1. Initial Engineering of the GA1-Fab Interface—

Protein G (PG) is an immunoglobulin binding protein that has been used for antibody purification by virtue of its affinity to the Fc portion of the molecule. PG also has weak affinity to the Fab framework (˜3 μM). The inventors had previously engineered an affinity improved Protein G variant (GA1) that bound specifically to a Herceptin Fab scaffold variant (E212S mutation in Fab constant light chain (Lc)) that could be utilized for applications that involved linking multiple copies of GA1 together to make multi-valent and bi-specific assemblages (23). Thus, the inventors sought to further develop the platform to facilitate building higher level modules that could incorporate multiple interchangeable Fabs in a plug-and-play fashion.

An important element of initial design was that the affinity matured GA1 would only bind to the specific E123S Fab Lc variants (Fab^{S}) that were designated parts of the assemblages, not any natural wild-type Fabs (Table 1) that were contained in endogenous IgGs or other sources in the experimental milieu. However, although the GA1-Fab^{S} complex had a 60-fold improved K_{d} of ˜50 nM compared to the wild-type (wt) Protein G domain, the binding is still characterized by fast dissociation kinetics that are not optimal for the desired non-equilibrium applications. Therefore, the inventors sought to further engineer the Herceptin variant scaffold (Fab^{S}) to have a significantly elevated affinity to GA1 accompanied with slow off-rate kinetics.

TABLE 1
Protein GA1-binding affinity of different
Fab Lc variants.

| FAB LC scaffold | K_{D} |
|---|---|
| (aa 123-127) | (nM) |
| GSLRS (SEQ ID NO: 26; selected) | 2.5 |
| SMLRS (SEQ ID NO: 56; selected) | 7.5 |
| AALKS (mutated) | 12 |
| SQLRT (SEQ ID NO: 258; mutated) | 8 |
| EQLKS (SEQ ID NO: 259; Hkappa) Fab^{□} | NB |
| EELQA (SEQ ID NO: 260; Hlambda) | NB |
| EQLTS (SEQ ID NO: 261; MKappa) | NB |
| EELET (SEQ ID NO: 262; Mlambda) | NB |

The initial affinity maturation of Protein G (PG) to produce GA1 involved phage display selections focusing on two points of contact with the Fab^{S} scaffold. [FIG. 2]. The first region was through the formation of complementary β-strands from PG (β2, residues 16-22) and residues 209-216 of the last β-strand of the heavy chain (Hc) of the constant domain of the Fab. The engineered interface includes all of the main chain hydrogen bonds observed in the original structures (24). Although, β-2 of GA1 contains several mutations that bury significant surface area at the protein interface, the overall affinity gain of these interactions is limited. The more noteworthy changes within GA1 accounting for the major affinity improvement occur at the C-terminal cap of the α-helix [FIG. 2], where the original residues 40-43 (⁴⁰NDNG⁴³ (SEQ ID NO: 266)) were substituted for ⁴⁰YVBE⁴³ (SEQ ID NO: 267) in the engineered GA1 variant. The helical cap of the engineered variant provides improved shape complementarity to interdigitate with the α-helix residues SQLKS (SEQ ID NO: 268) (residues 123-127) connecting β-strands of the Lc domain of the Fab.

2. Affinity Maturation of the Binding Interface of Fab^{S} Lc to Protein GA1—

While compared to wt-PG, GA1 produced a significant affinity boost in binding Fab^{S}, it still was not optimal for the engineered modules the inventors envisioned. To further improve the affinity and the off-rate kinetics, it was hypothesized that a stepwise phage display approach was the best way to further increase the Fab^{S}-GA1 affinity. That is, as described above, GA1 was produced from phage display selections against the original Fab^{S} scaffold. In the stepwise selection scheme, the process is reversed; Fab^{S} is affinity matured against GA1. To perform phage display on the complementary surfaces on the Fab^{S} scaffold, the inventors designed a phage display library focusing on residues 123-127 of the Fab^{S} Lc, since it formed the most extensive contact with GA1, as described above [FIG. 2]. Different levels of diversity were introduced into each of these positions depending on the amino acid character of the parent residue (see Materials and Methods). This library had a theoretical diversity of roughly 10⁷ Lc-scaffold variants. A protocol for target immobilization through a cleavable SNAP-tag was applied to all Fab selections as has been described (25). Using this approach, the C-terminally SNAP-tagged GA1 was biotinylated through the SNAP self-modifying activity using commercially available SNAP-Biotin enabling capture by streptavidin-coated magnetic beads during selection.

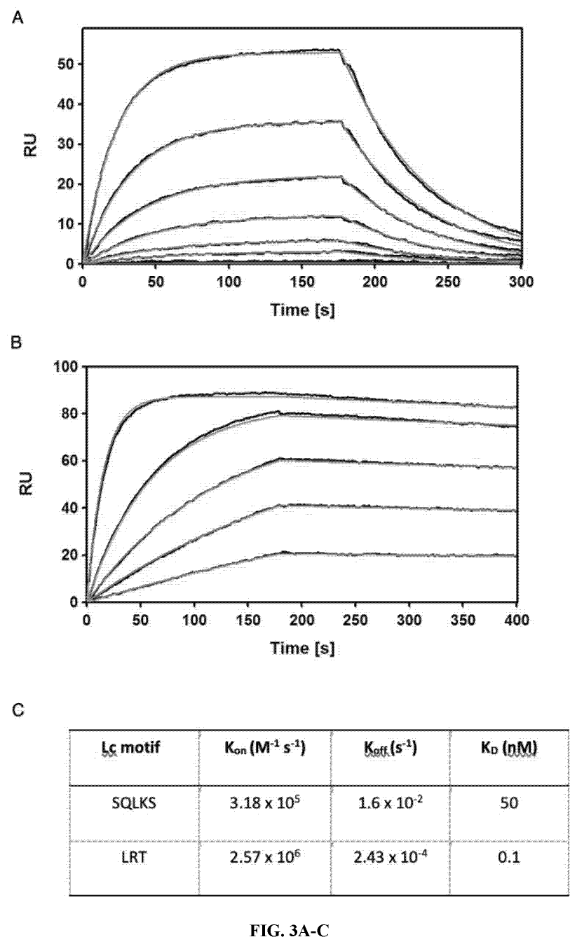

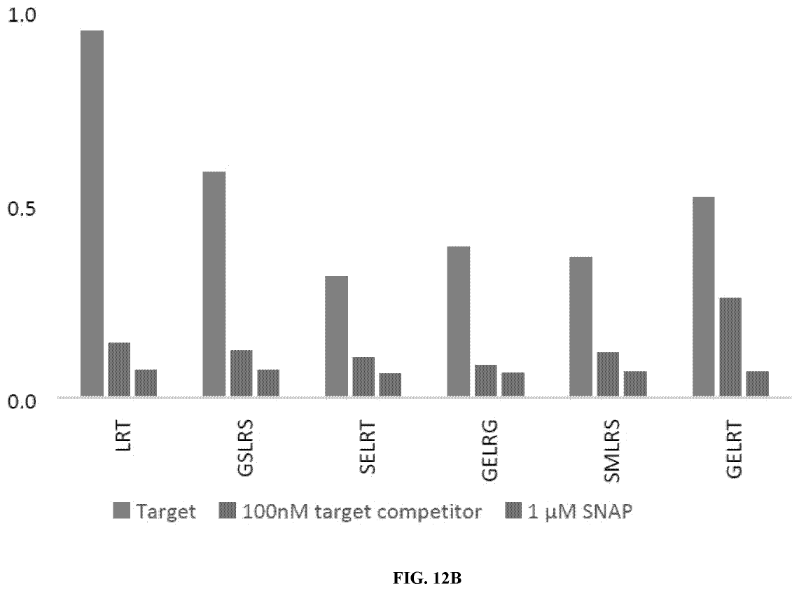

Five rounds of selection were performed using the Fab^{S} Lc library. To introduce additional binding stringency, the concentration of the antigen was systematically reduced with each round of selection starting at 200 nM during the first round and ending with 1 nM in round 5. Phage ELISA was performed on 96 clones resulting in identifying six unique Fab^{S} variants. Notably, sequencing revealed that all six of the variants contained a K126R substitution [FIG. 12]. Two of the variants resulted in a 5-10 fold improved GA1 binding affinity as determined by surface plasmon resonance (SPR) (GSLRS (SEQ ID NO: 26) and SMLRS (SEQ ID NO: 56), Table 1). Importantly, a single variant containing a serendipitous two amino acid deletion, ΔΔLRT (Fab^{LRT}), produced significantly superior binding characteristics. The replacement of the original SQLKS (SEQ ID NO: 268) sequence of Fab^{S} with ΔΔLRT produced a K_{d} of 100 pM and a slow dissociation rate. Overall, compared to the Fab^{S}, the Fab^{LRT} improved the affinity to GA1 by ˜500-fold [FIG. 3C]. This deletion mutation did not affect Fab stability or expression. The inventors speculate that the deletion may have occurred during the synthesis of randomizing DNA oligonucleotides.

To evaluate the relative importance of the conservative mutations K126R and S127T relative to the two amino acid deletion at positions 123-124, two variants were constructed. The first included the deletion, but replaced the Arg with the wild type Lys (ΔΔLKT). The second one contained the wild type residues at positions 123-124 followed by LRT (SQLRT (SEQ ID NO: 258)). SPR analysis determined that both these variants had intermediate affinities (12 nM and 8 nM, respectively) in the range between GA1 (50 nM) and ΔΔLRT (0.1 nM) [Table 1]. These data, together with the fact that all selected scaffolds contained affinity-improving K to R substitution, suggest direct and significant involvement of the Arg in the enhanced interactions with GA1.

3. The Structure of GA1-Fab^{LRT}—

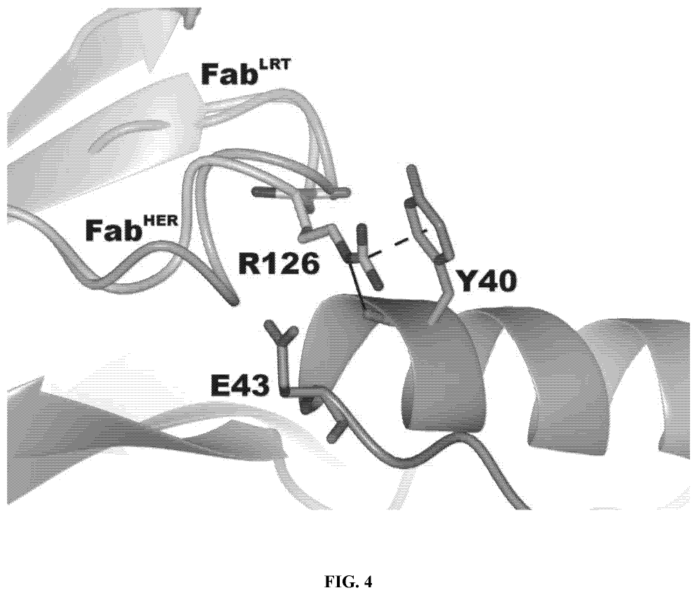

The crystal structure of the Fab^{LRT}-GA1 complex was determined to gain structural insights into how the ΔΔLRT mutation enhances the binding affinity between the Fab and GA1 to the extent that it does [Table S1]. The complex crystallized in space group P3221 with two Fab^{LRT}-GA1 complexes in the asymmetric unit. The average root-mean-square deviation (RMSD) between the two Fab-GA1 complexes is ˜0.2 Å (over 211, 220, and 56 Cα atoms of the Fab Lc, Fab He and GA1, respectively). The GA1-Fab^{LRT} interface is formed through two sets of contacts that bury ˜560 and 160 Å² of the Fab's He and Lc, respectively. The first contact is through the formation of an antiparallel β-strand configuration that includes main chain H-bonds between residues 16-22 of GA1 β2 and residues 221-227 of Fab He βC. A similar H-bonding arrangement was reported in the structure of a wild-type PG-Fab complex (24). A second and more extensive set of contacts involves the C-terminal α-helical cap of GA1 and Fab residues comprising 137-140 of the He and 123-127 in the Lc, which includes the ΔΔLRT motif [FIG. 2]. Notably, the ΔΔLRT motif mates with the residues of GA1 (⁴⁰YVHE⁴³ (SEQ ID NO: 267)) that were involved in GA1's affinity maturation from PG to GA1. The structure shows that the loop containing the deleted residues in the ΔΔLRT motif induces a conformational change that positions the guanidium group of R126 to pack against the aromatic ring of Y40 of GA1 resulting in the formation a cation-π interaction [FIG. 4]. Further, the guanidinium group forms a H-bond with the carbonyl of Y40. V41 of GA1 forms hydrophobic interactions with F139 of Fab He RA. Additionally, H42 of GA1 is buried at the He Fab interface, where its Nε2 nitrogen forms a H-bond to the main chain nitrogen of the V129. The H-bonding potential at this position appears to be conserved, as all phage display variants isolated have either His, Asn or Gln at this position. E43 is exposed to the solvent and protrudes into the cavity created by the two deletions at the ΔΔLRT motif.

4. GA1-Fab^{LRT} Protein Complementation Assays: Principle and Components—

As the model system for the proof of principal of the plug-and-play GA1-Fab^{LRT} concept, the inventors devised a protein complementation assay (PCA) based on the well-established proximity-driven refolding/reactivation of the TEM1 β-lactamase (BL) split enzyme system (26). In this assay, the two separate fragments of the BL enzyme are attached through a linker to the two different targets that are to be evaluated for proximity. If the targets are in close vicinity, then the fragments can associate to form an active enzyme state. This can be evaluated readily by introducing a fluorogenic BL substrate that provides a distinct readout. The format generally requires that the individual complementary fragments be genetically fused by means of a linker to one or the other of the potential interaction partners. The linker lengths can be adjusted to fine tune the complementary efficiency. However, this requires multiple genetic fusions that can be cumbersome and time consuming.

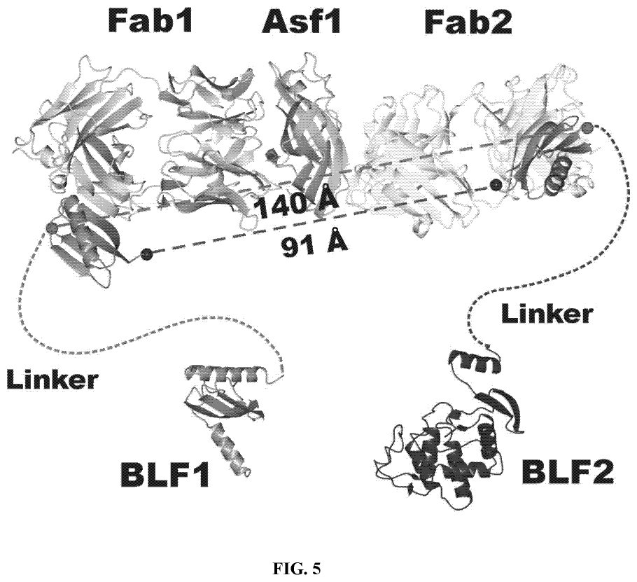

To circumvent the issues involving serial genetic fusions, the inventors developed a system that exploits the high affinity of a Fab^{LRT} to GA1. Our test case involves using complementation in the form of a canonical sandwich assay. The strategy is to express and purify two GA1 fusions with one or the other of the two complementary BL fragments (BLF): N-terminal fragment-residues 26-196 and C-terminal fragment-residues 198-220. In order to bring the BL fragments in proximity allowing for BL association and refolding at low concentrations in this type of antigen-detection assay, the GA1 modules of complementary BLF fusions are associated separately with two LRT scaffold Fabs that bind the antigen at different epitopes. Then, upon addition of the antigen, simultaneous antigen-binding of these Fabs results in BL refolding and activation [FIG. 5]. The induced BL activity is detected by the increase in fluorescence signal upon addition of Fluorocillin Green, a fluorogenic BL substrate. Notably, the Fab-binding GA1 module genetically fused to BLFs could serve as a potent non-covalent linker between the BLFs and any number of interchangeable Fab^{LRT} molecules, laying the basis for plug-and-play opportunities.

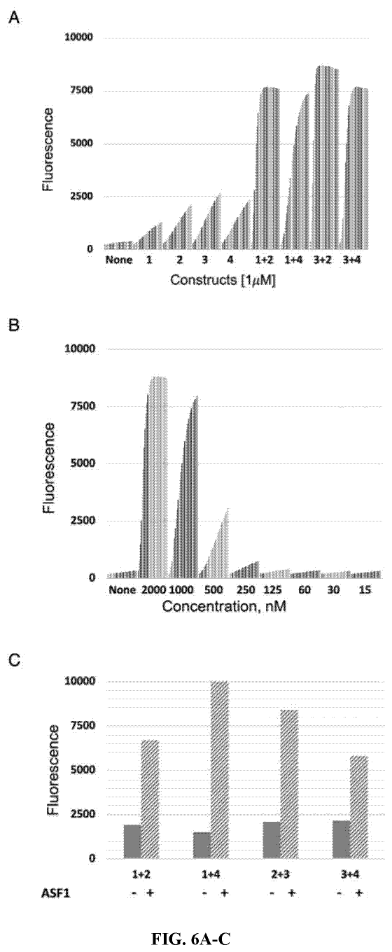

To optimize the system and explore the different options, the inventors constructed and produced 4 fusions of combinations of the N-terminal (BLF1) and C-terminal (BLF2) fragments of BL connected to GA1 by a Gly-Ser linker of about 30 residues. Next, the inventors demonstrated that BLF-GA1 fusion constructs in the absence of antigen were capable of BL reconstitution by testing them at 1 μM concentration in the β-lactamase assay with its complementation partner [FIG. 6A]. When mixed together at different concentrations, the pair: 1 and 4 (BLF1 fused to the C-term of GA1 and BLF2 fused to the N-term of GA1) showed the lowest spontaneous activity level at 1 μM [FIG. 6A]. This pair was then used to establish the background level at concentrations between 2 μM and 15 nM. This showed that BL activity in the absence of antigen was triggered at concentrations above 500 nM. Thus, the inventors chose a concentration of 250 nM [FIG. 6B] that was well below this threshold as the baseline for the antigen-detection conditions, since it was the highest concentration that displayed minimal background activity in the absence of antigen.

As an initial model for the sandwich assay development, the inventors chose as the antigen a small, 158 amino acid histone chaperone protein, Asf1. Two Fabs (11E and 12E), shown to be binding to orthogonal epitopes of the protein, had previously been generated (27). A crystal structure of the Asf1 with these two Fabs indicated that they bound on opposite faces of the protein (20). To establish possible linker lengths that might work in this system, further examination of the superimposed crystal structure model of the tripartite ASF1:12E:11E complex with GA1 bound to each Fab, indicated a ˜100-150 Å distance range between the termini of the two GA1 molecules [FIG. 5]. Two competing criteria were considered in selecting effective linker lengths. First, the length should not be too long as to diminish the local concentration effect. However, perhaps more importantly, effective linker lengths cannot be estimated by measuring directly between point A and B. There has to be built-in excess to take into consideration their inherent flexibility and the fact that the Ramachandran plot has to be adhered to in the process.

Taking these issues into account, the inventors surmised that a 30 amino acid Gly-Ser linker was a reasonable compromise between these requirements. Indeed, in pilot experiments where each of the pairs of complementary BLF-GA1 fusions were pre-mixed separately with 11E or 12E Fabs having LRT mutations, a significant increase (up to 10-fold) in the fluorescent signal was produced upon addition of an equimolar amount of Asf1 [FIG. 6C]. Although all combinations worked, the inventors found as before that the 1-4 pairs reproducibly produced the best signal-to-noise ratio.

5. Dual Epitope Fabs Against NP^{CT}EBOV and MT ZIKV

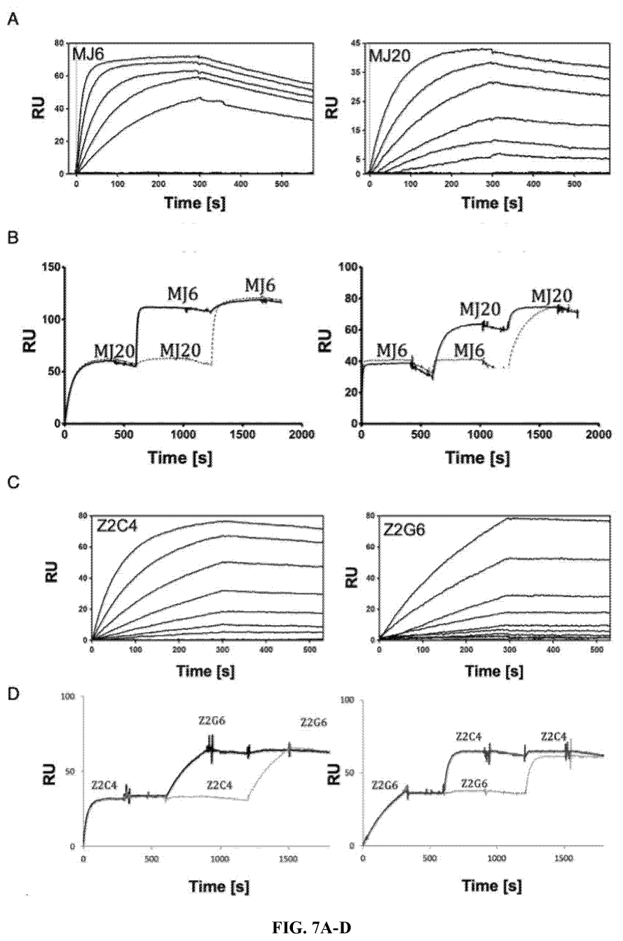

To further develop the GA1 fusion platform and apply it to antigen detection in the systems with unknown structural organization, the inventors chose two viral protein antigens where previously generated Fabs were available. The first was the 98 residue C-terminal domain of the Zaire strain of Ebola virus nucleoprotein (EBOV NT^{CT}). The second was the 261 residue, N-terminal methyltransferase domain of the Zika virus bifunctional NS5 enzyme (MT ZIKV). From the pool of Fabs selected against NT^{CT} from five known major Ebola virus strains [Table S2], epitope binning revealed two distinct epitopes. The major epitope was highly dominant, while only a single Fab (MJ6) was found that bound to a second independent epitope. From the group of major epitope Fabs, MJ20 was selected as a representative binder and was used in subsequent studies. Using a dot blot analysis, Fab pairs, MJ20 (major epitope) and MJ6 (minor epitope), were shown to bind simultaneously to EBOV NT^{CT}. The binding kinetics of the pair were subsequently determined by SPR analysis indicating affinities of 0.7 nM (MJ6) and 3.4 nM (MJ20), with dissociation rates of 1.0×10⁻³ sec⁻¹ and 6.1×10⁻⁴ sec⁻¹, respectively [FIG. 7A]. Further, it was shown by SPR that consecutive injections of Fabs MJ6 and MJ20 resulted in an approximately two-fold increase of the mass signal compared to single injections of either of them or two consecutive injections of the same Fab [FIG. 7B]. This confirmed that MJ6 and MJ20 are capable of binding simultaneous to non-overlapping epitopes of EBOV NT^{CT} without affecting the affinities of either Fab.

Among Fabs selected against MT ZIKV, using the procedures described above, a pair of Fabs, Z2C4 and Z2G6, was found to bind to non-overlapping epitopes [FIG. 7C-D]. These Fabs exhibited K_{DS} of 0.7 nM and 1.7 nM and dissociation rates of 2.8×10⁴ sec⁻¹ and 9.2×10⁻⁵ sec⁻¹, respectively [FIG. 6E]. Thus, the inventors confirmed that pairs of Fabs for both systems (EBOV NT^{CT}: MJ16 and MJ20; MTZIKV: Z2C4 and Z2G6) possessed the desirable antigen-binding characteristics for our GA1-BL detection system (high affinity, slow dissociation rate, independent binding to the antigen molecule) and could be introduced into formats to test their abilities in the plug and play proximity assays.

6. Detection of Viral Proteins: NP^{CT} EBOV Zaire and MT ZIKV.

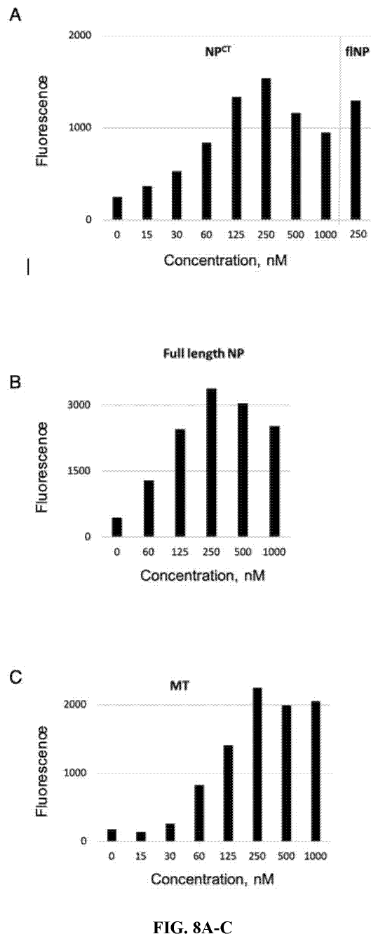

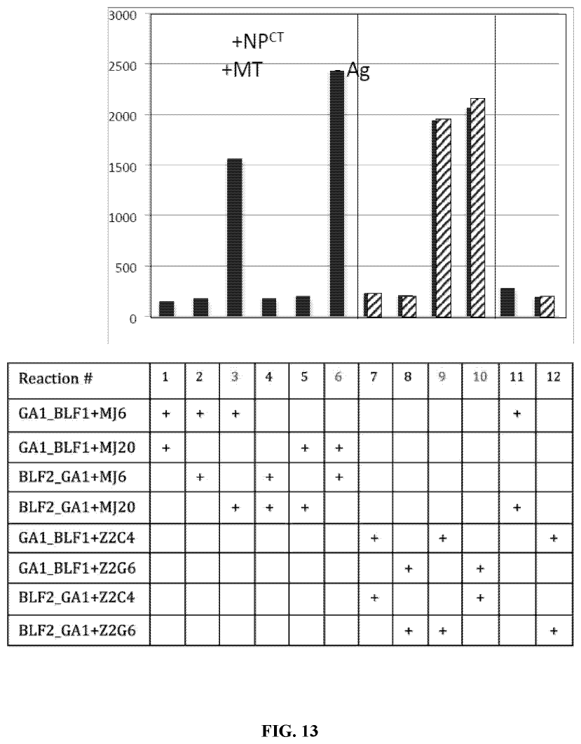

A challenge for the EBOV and ZIKV systems was the absence of information about the position of the epitopes of the Fabs that were being employed in the proximity assay. Only the crystal structure of EBOV NT^{CT} with one Fab, MJ20, had been solved (25). As with the Asf1 system, the inventors employed a 30 residue Gly-Ser linker to connect GA1 to the BLFs. To test this system in the context of the Fab^{LRT} components (MJ6 and MJ20) and the complementary fusions between protein GA1 and the BLFs, the inventors individually premixed the Fabs with each of the complementary fusions at a final concentration of 250 nM. The BL activity induced upon addition of equimolar 250 nM NP^{CT} revealed a preferential performance of the GA1_BLF1+MJ6 and BLF2_GA1+MJ20 premix out of the two possible active combinations [FIG. 13]. Notably, reversing the format, that is, matching BLF2 with MJ6 and BLF1 with MJ20, reduced the activity by about 40%, suggesting some sensitivity between the matched pairs. Negative-control mixtures, containing the same BLF-fusion or the same Fab component in the pre-mixtures, did not show any significant activation upon antigen addition [FIG. 13]. Titration of NP^{CT} into 250 nM of the combination of GA1_BLF1+MJ6 and BLF2_GA1+MJ20, resulted in a detectable fluorescent signal at 15 nM NP^{CT}, which increased linearly over the range from 15 to 125 nM with a distinct maximum at 250 nM [FIG. 8A]. A reduction of the signal observed at NP^{CT} excess, most likely was caused by a breakdown of the stoichiometry at high antigen concentration.

Next, the inventors asked whether NP^{CT} in context of the full-length EBOV NP Zaire could be detected by the above system with comparable efficiency, since the additional N-terminal NP domain might create a steric hindrance for Fab binding or BL refolding. However, the NP^{CT} domain contained in the full-length NP Zaire protein was readily detected, as measured by an increase in BL activity similar to the NP^{CT} antigen alone making this assay applicable to full-length NP and potentially to EBOV detection in biological samples containing the lysed virus [FIG. 8A, B].

To further demonstrate the plug-and-play capabilities of the platform, the inventors applied the same BLF-GA1-fusion constructs to detect MT ZIKV using the FAB^{LRT} format with Z2C4 and Z2G6. The results obtained for MT ZIKV were congruent with the findings of the EBOV detection system [FIG. 8C]. At a 250 nM concentration of the GA1_BLF1+Z2C4 and BLF2_GA1+Z2G6 combinations, the limit of antigen detection for both systems appeared to be roughly 30 nM, which falls within the range published for laboratory-performed Ebola-detection ELISA assay (28) and the maximum of the signal was achieved at the equimolar 250 nM concentration of the BLFs and the antigen.

7. A Novel Plug-and-Play Bi-Specific T-Cell Engager Immuno-Reagent

Bi-specific T-cell Engagers (BiTEs) have recently emerged as an important class of immuno-therapeutic assembly (29). BiTEs are molecules that are engineered to engage an activated T-cell through one binding arm and to attach it to a cell surface target on an antigen presenting cancer cell (APC) through its second arm (30). This engagement leads to T-cell dependent cell death of the cancer cell. BiTEs using several formats have been developed and successfully deployed (31-34). The most prevalent formats to induce engagement between the two cells are: i) bispecific antibody where one arm recognizes the T-cell and the other the APC, and ii) two cell-directed single-chain Fvs attached by a flexible linker. Each of these formats has its strengths and weaknesses, but neither has the versatility provided by GA1-Fab^{LRT} constructs described below.

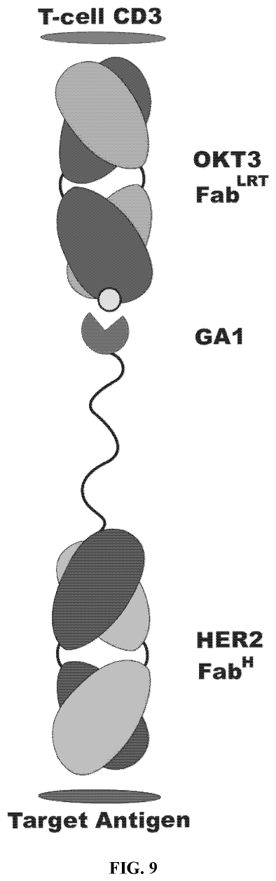

The designed bi-Fab constructions are based on a GA1-Fab^{LRT} concept and are bi-specific with adjustable linker lengths between the two antigen binding modules [FIG. 9]. A number of such fusion constructs were engineered with different linker lengths (from 3 to 73 aa long) between GA1 and the C-terminus of the He of the Herceptin Fab scaffold with a specificity directed at one of the target antigens. The Herceptin scaffold differs from Fab^{S} by a single amino acid in that it has the wt kappa Lc with Glu at position 123, rather than Ser, as is the case for Fab^{S}. This scaffold is referred to as Fab^{H}. A fusion construct with 13 residue linker (GGSGSAGSGGAGA—SEQ ID NO:124) was used for the proof of principal described below. The concept is that a Fab(1)^{H}-linker-GA1 fusion that binds to antigen target 1 can be combined with a Fab^{LRT} that binds antigen target 2 (Fab(2)^{LRT}) [FIG. 9]. This forms a noncovalent entity consisting of Fab(1)^{H}-linker-GA1-Fab(2)^{LRT}. These modules are referred to as “bi-Fab” BiTEs. Such constructs allow easy cloning of any desired Fab CDRs into the Fab scaffold (in this example, Fab^{H}), the resulting GA1-Fab(target 1) fusions can be efficiently produced through E. coli periplasmic expression. Importantly, GA1 binds preferentially to Fabs containing the LRT motif with the Fab^{S} scaffold and does not bind to Fabs with wt kappa Lcs (Fab^{H}) (Table 1), therefore, there is no “self” association within the Fab^{H}-linker-GA1 component of the module.

To test the bi-Fab module in a biological application, the inventors chose to construct a BiTE that would induce engagement between a cell that had an overexpressed cell surface cancer marker through one arm and a cytotoxic T-cell through the other. Thus, for the first arm (Fab^{H}), the inventors chose to target the specific APC marker, Her2, which is highly over-expressed on the surface of many breast cancer cell lines. For the Fab(target 2)^{LRT} arm the inventors chose a humanized Fab version of an antibody that binds the CD3 component of the T-cell receptor complex and activates it [37, 38]. The inventors hypothesized that the tight noncovalent link between T-cells and tumor cells created by these bi-Fab immuno-reagents would induce robust immunological-synapse formation, leading to T-cell activation and secretion of cytokines and cytotoxic granules resulting in lysis of the tumor cell.

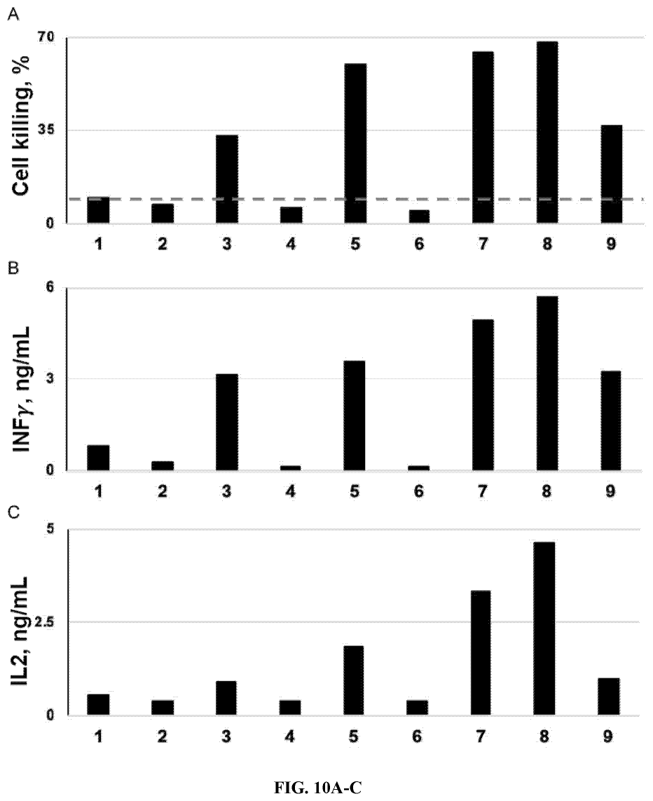

Fab^{S} of the bi-Fab was derived from the α-Her2 trastuzumab antibody. The Fab(2)^{LRT} component was based on introducing the CDRs of either of the widely used CD3 antibodies, OKT3 or UCHT1 into the LRT engineered Fab scaffold. Thus, either CD3 Fab can be interchangeably plugged into the GA1 unit. The full bi-Fab module was assembled and assessed for activity in a redirected tumor-cell killing assay. The assay has three readouts: i) the activity of a cytoplasmic enzyme, Lactate Dehydrogenase (LDH), released into the medium upon cell lysis, ii) interleukin IL2 and iii) interferon γ production by T helper cells. As the source of Effector T-cells, the inventors used isolated human PBMCs. The target cells were from Her2-positive SKBR3 human breast-cancer cells. Addition of bi-Fabs in several different active combinations to PMBC-SKBR3 co-cultures at the optimal 50 nM, corresponding to early saturation concentration point, resulted in robust cell killing (up to 70%). Furthermore, these conditions led to prominent IL2 and IFNγ release [FIG. 10]. Notably, these levels somewhat surpassed those of the positive-control bi-specific antibody, representing hOKT3 Fab-hHer2 scFv genetic fusion [FIG. 9]. Importantly, the functional readouts were similar when the format was switched; that is, when the anti-CD3 Fab is introduced into the construct as the fusion with GA1 and the Her2 is the Fab^{LRT} component [FIG. 9]. Thus, the activity of the bi-Fab is independent of organization of the Fab components. All activities were abolished upon introduction of CDR mutations eliminating CD3 binding within the CD3 Fab^{LRT} element. As expected, the assembly of the functional non-covalent bi-Fab was dependent upon the genetic fusion of GA1 to Fab^{H}, as no detectable activity was observed when the proteins (Fab^{H}, Fab^{LRT}GA1) were added as three separate unlinked entities. These results demonstrate the utility of the high affinity GA1-Fab^{LRT} binding pair for the facile construction of bi-specific immuno-reagents. Such a strategy should prove especially useful when large numbers of antibodies need to be screened in combination, streamlining the time and resource-intensive expression and purification of bispecific molecules.

B. Discussion

The inventors have described the development of a platform that facilitates the coupling of Fab-based affinity reagents in multi-valent and multi-specific formats. The core of the technology is a module of Protein-G (GA1) that had been affinity matured by phage display mutagenesis to bind tightly to variant Herceptin Fab (Fab^{S}) scaffold (23). The interaction between Fab^{S} and GA1 was further enhanced by a subsequent affinity maturation of the Fab^{S} scaffold against GA1. Interestingly, the highest affinity Fab^{S} variant (Fab^{LRT}) contained a serendipitous two amino acid deletion within the region of five amino acids that were diversified in the phage display library. Together, this tandem mutagenesis approach resulted in an affinity of 100 pM between Fab^{LRT} and GA1, which was over 500-fold tighter than that between the starting Fab^{S} and the wild-type Protein G. While this is not a covalent interaction, our results indicate it is clearly of sufficient affinity for the applications that the inventors investigated.

The impetus for developing this platform was to overcome myriad limitations of traditional antibody-based affinity reagents. Antibodies have evolved structures and specificities optimized for in vivo immune recognition, not as tools for cell biologists. Recombinant technology has allowed for engineering scaffolds and optimizing specificities through relatively straightforward processes (2). This has enabled generating versatile assemblages with bi-specific capabilities, allowing simultaneous recognition of multiple antigens. Nevertheless, these assemblages are generally constructed with particular pairs of targets in mind. If one or both of the targets change, then a new construct has to be designed, built and optimized. Thus, the inventors endeavored to simplify the process by developing a tool kit that allows facile exchange of affinity modules in a plug and play fashion utilizing the ultra-high affinity of the Fab^{LRT}-GA1 pair.

The first system the inventors investigated involved the use of an enzyme complementation format to evaluate the properties of the GA1 fusions in the context of a sandwich assay. This requires two non-overlapping epitopes on the antigen so that two independent Fabs can bind simultaneous. Several constructs were made; the first comprised the N-terminal fragment of beta lactamase (BLF1) fused to a 30 amino acid linker with GA1, the second was identical except that the C-terminal fragment (BLF2) was attached through a similar linker to a GA1 module [FIG. 5]. Further, the formats were expanded whereby linkers were fused to either the N- or C-terminal ends of GA1, introducing additional spatial variation. Each of these fusions was then mixed with one of the Fabs in a variety of combinations. A key concern of ours initially was that since the Fab^{LRT}-GA1 interaction is not covalent there might be exchange between components that might compromise the efficiency of the assay. However, the inventors determined that with the 100 pM affinity between the pair, no measurable interchange occurs within the timeframe of the experiment.

Further, it might be assumed that since this complementation assay was being performed using a small protein, Asf1, it does not offer a challenging system because the Fab binding sites are close together. However, the converse is actually true. The two epitopes on Asf1 are on directly opposing faces of the protein, effectively orienting the GA1 binding sites on the Fab scaffold such that the Gly-Ser linkers point in opposite directions [FIG. 5]. Anyone who has built a molecule model realizes that it is difficult to “turn a corner” in an efficient way while still adhering to reasonable conformational energies. That is why the inventors added the option of fusing the linker to either the N- or C-terminal end of GA1 to increase the potential spatial disposition of the BL fragments. Indeed, it was found that in the case of the Asf1 sandwich assay there was some bias with regard to how the fragments were hooked up, but this did not carry over to other systems where pairings did not appear to matter. While no attempt was made to optimize the system, the general overall success of the complementation-sandwich assay demonstrated that the system had enough inherent flexibility to suggest it could be broadly utilized for not only sandwich assays, but also other types of proximity assays. One can imagine a scenario where Fabs could be made to multiple cell surface targets. The Fabs could then be hooked up and profiled in high throughput to identify proximal cell surface neighbors.

A different format for the GA1 fusion was used in the development of the bi-Fab BiTE construct (30, 31, 34). The concept of BiTEs has been developed to connect and bring together two different cell types, one being a cytotoxic T-cell and the other a tumor cell (32, 33). A BiTE can take several different forms, but the basic construct is comprised of two linked antibody-based moieties, one targeting a component of the T-cell receptor on the T-cell and the other targeting an over-expressed surface antigen on the tumor cell (APC). Adding the BiTE initiates extensive crosslinking of the cells leading to T-cell activation and subsequent tumor cell death. The effectiveness of the construct depends on multiple factors ranging from target density and binding potency to their linker length (35, 36). Importantly, simply linking a T-cell to an APC does not induce cell death, the binding component of the T-cell has to target certain components of the T-cell receptor, most notably CD3 (37, 38). The inventors designed a bi-Fab BiTE that could function as a cassette that allows facile interchange of Fabs directed at different cell surface targets. The basic component was a Fab-GA1 fusion that was directed at the HER2 antigen that is overexpressed on SKBR3 cells, a breast cancer cell line. Connecting GA1 to the Fab (Fab^{H}) is accomplished by fusing it by means of a 13 residue linker to the C-term of the He of the Fab. Adjusting of the linker length is straightforward and it does not affect the expression or stability of the basic cassette. Adding a Fab^{LRT} to this modality results in formation of the full length BiTE (Fab^{H}-linker-GA1-Fab^{LRT}). In the test case, the Fab^{LRT} was a humanized Fab version of either OKT3 or UTCH1, which are highly validated antibodies that activate T-cells through their binding to CD3 (37, 38). Either of these CD3 binding LRT Fabs could be interchangeably introduced into the HER2 Fab^{H}-GA1 module. Adding the assembled BiTEs to a mixture of PBMCs (which contain cytotoxic CD8 and CD4 cells) and SKBR3 breast cancer cells elicited readouts that verified induction of cancer cell death.

Given the ease of interchanging Fab^{LRT} in the BiTE module, one might envision a high throughput campaign to profile BiTE efficiencies targeting many different cancer specific cell surface antigens. To do this most efficiently, the format described above should be reversed. The inventors showed that the “polarity” of the bi-Fab makes no difference to its effectiveness; that is, which target Fab is included in the fusion with GA1 or as the Fab^{LRT} component [FIG. 10]. The Fab-GA1 fusion would be constructed to contain the CD3 binding Fab, OKT3 or UTCH1; this is an easy cloning step. Then the Fab^{LRT} could be a Fab targeting any number of cell surface cancer markers. In this format there is no need to make constructions for each bispecific pair, each BiTE can be rapidly assembled in a plug and play fashion. Further, it is easy to change the linker lengths and even to put multiple GA1 modalities on the linker to exploit possible avidity effects.

To extend the system even further, one can imagine that by using the tandem phage display approach new sets of distinct high affinity Fab-GA1 interactions could be engineered. This could expand the distinct specificity of the fusion modules past being bi-specific to tri-specific or even tetra-specific. While this remains a future goal, one can imagine the unique types of experiments that would be in reach with this technology in hand.

C. Materials and Methods:

1. Protein Cloning, Expression and Purification—

The sequences of all the constructs used are provided in Table S3. The open-reading frames (ORFs) encoding the C-terminal domain of Nucleoprotein (NP^{CT}) from Zaire (EBOV), Reston and 3 other strains of Ebola virus, full-size EBOV NP and Zika virus Methyltransferase (MT ZIKV) optimized for bacterial expression, were gifted by Dr Z. Derewenda, University of Virginia. To serve as targets for phage selections, these viral ORFs as well as ORFs coding for yeast histone chaperone protein-Asf1 (27) and protein GA1, an engineered high-affinity Fab-binding variant of Protein G domain C3 (23), were cloned using Sma1 site into pEKD40 with the cleavable N-terminal SNAP-tag and the C-terminal 6×His tag (SEQ ID NO: 269). pEKD40 is a derivative of pSNAP-tag (T7)-2 vector (NEB) that was modified with the thrombin-cleavage site at the C-terminus of the SNAP-tag followed by Sma1 site and a C-terminal 6×His tag (SEQ ID NO: 269) added for enabling of protein purification. For the β-lactamase split enzyme proximity applications, the viral proteins and Asf1 were cloned without SNAP-tag using Xho1-BamH1 sites of the pHFT2 version containing TEV-cleavable N-terminal 10×His tag (SEQ ID NO: 270) (39). The same strategy using pHFT2 vector was applied for cloning of four of the BLF_GA1 fusion constructs comprised of one of two TEM-1 β-lactamase (BL) complementation fragments: BLF1, aa 26-196 bearing a M182T mutation (26) or BLF2, aa 198-290, connected to the N- or C-termini of GA1 by roughly 30 aa-long GS linkers. Selected Fabs and Fab-scaffold variants from phage clones were cloned into Sph1 sites of pSFV4 expression vector (found on the world wide web at thesgc.org/sites/default/files/toronto_vectors/pSFV4.pdf) using InFusion HD cloning kit (Clontech) as recommended.

To obtain Her2, OKT3 and UCHT1 Fabs (Table S3), their humanized CDR-containing regions (synthesized as gBlocks by IDT) were cloned into pSFV4 using Nco1 and SgrA1 sites. To improve bacterial expression of the OKT3 Fab, the Cys in CDR H3 of OKT3 was substituted with Ser. Genetic fusion of GA1 to the C-terminus of Fab^{H} (Lc S123E) variants was achieved by cloning of GA1 containing an N-terminal 13 aa long linker (GGSGSAGSGGAGA (SEQ ID NO: 124)) into SgrA1 of pSFV4. Fab^{LRT} (ΔΔLRT) and Fab^{H}(S123E) distinguishing mutations were grafted into Fab^{S} Lc at aa positions 123-127 (SQLKS (SEQ ID NO: 268)) using quick change site-directed mutagenesis.

Expression of 6× and 10×His-tagged proteins (SEQ ID NOS 269-270, respectively) was induced by addition of 1 mM IPTG to 1 L E. coli BL21 (DE3) cell cultures grown in 2×YT medium to a mid-log phase (0.4-0.6 OD₆₀₀). After the overnight incubation at 18° C. (250 rpm), harvested cells were sonicated in buffer A: 50 mM Tris-HCl, pH 8.0, 150 mM NaCl and centrifuged. Then, either native or denaturation purification protocols were applied for protein purification depending on their solubility. Soluble 6× and 10×His-tagged proteins (SEQ ID NOS 269-270, respectively) were purified from the cleared supernatants by TALON (Clontech) Immobilized metal affinity chromatography (IMAC) using a standard native-condition procedure and elution by 100 mM imidazole in buffer A. The insoluble 10×His-tagged (SEQ ID NO: 270) BLF_GA1 fusion proteins were extracted from the pellets by 6M Gua-HCl in buffer A with 0.3 mM TCEP and purified on TALON resin using a denaturation-condition protocol and on-column renaturation achieved by 6 washes of the column by the sequential 2-fold 6M Gua-HCl dilutions in buffer A and a final buffer A wash. The renaturated BLF_GA1 fusion proteins eluted with 100 mM imidazole in buffer A were immediately diluted with buffer A in order to lower their concentration to 0.5 mg/ml (or less) to prevent them from precipitation. These fusion proteins were never frozen and were stored on ice.

Fabs and Fab_GA1 fusion proteins were expressed in the periplasm of E. coli BL21 cells for 4-5 hour at 37° C. after induction by 1 mM IPTG at 0.8-1.2 OD₆₀₀. The cells were harvested by centrifugation and sonicated in 50 mM phosphate buffer, pH 7.4, 500 mM NaCl. The centrifugation-cleared sonicates were applied to one or the other affinity column depending on the LC scaffold of the Fab: Fab^{S} and Fab^{LRT} variants (possessing high affinity toward protein GA1) were purified on ProteinGA1 resin created in the lab as described (23) using SulfoLink Coupling Resin (Thermo Scientific), while Fab^{H}_GA1 fusions lacking GA1-binding affinity were purified using Protein A resin (Genscript). In both cases, Fab variants and Fab_GA1 fusions were eluted from the column by 0.1 M glycine, pH 2.6, and neutralized with aliquots of 1 M Tris-HCl pH 8.5. For short-term storage, Fabs and Fab fusions were kept at 1 mg/ml on ice.

2. Phage Display Selection Protocol—

A). SNAP-tagged target protein immobilization—The selection strategy for Fab generation was previously described in (23, 27, 40). Purified SNAP-tagged target proteins were SNAP-biotinylated at 20% excess of SNAP-Biotin (NEB) in the presence of 0.3 mM TCEP for 15 min at 37° C., followed by the binding onto streptavidin-coated paramagnetic beads (Dynabeads M-270, Invitrogen) using a standard protocol. 1 to 3 selections were performed with each of the SNAP-biotinylated Target proteins.

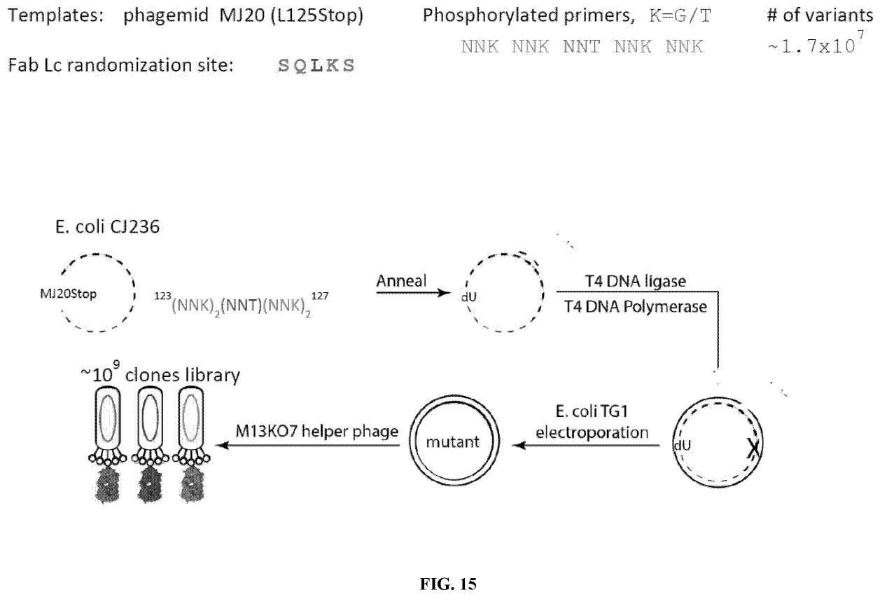

B). Phage display libraries—The Phage M13 Fab library, containing CDRs randomized at a diversity of >10¹⁰ in a variant of the human Fab^{H} scaffold, Fab^{S}, featuring a single aa substitution in Lc, E123S, Hc, C-terminally fused to the M13 minor coat protein pIII, was used for sAB selection against various antigens. Another phage library was created for selection of Fab Lc scaffold variants against SNAP_GA1 as a target protein, using the strategy previously published (41). To that end, five residues in Fab^{S} light-chain scaffold that interact with GA1 were chosen for hard randomization (FIG. 15): DNA encoding aa 123-127 (SQLKS (SEQ ID NO: 268)) in Lc MJ20 phagemid was replaced for NNK NNK NNT NNK NNK (SEQ ID NO: 263) (K standing for G or T: NNK covers 32 codons for all 20 aa and TAG Stop codon) using Kunkel mutagenesis protocol (40).

C) Library sorting procedure—Three to five rounds of phage sorting (depending on the selected phage specificity achieved) were performed at room temperature as described earlier (23, 27) with some modifications. For the first round, 200 μL (original volume) streptavidin-coated paramagnetic beads (Promega) with immobilized SNAP-biotinylated target proteins were incubated in 1 mL phage library (10¹¹ cfu) at 200 nM final target-protein concentration for one hour at RT. The beads were washed manually 2 times using a magnetic stand and added to log phase E. coli XL1-blue (Stratagene) for 20 min. Then M13K07 helper phage was added to final concentration of 10¹⁰ pfu/mL for the overnight phage amplification. For all subsequent rounds, the amplified phage was precipitated twice in 20% PEG/2.5M NaCl, and placed at 1-2 OD₂₈₆/well into an automated Magnetic Particle processor (KingFisher 700, Thermo Scientific). The phage was captured from 100 μL well solution containing target-coated beads (2 μL original bead volume/well) in the presence of 1 mM 06-Benzylguanine-blocked SNAP protein as a competitor. The final concentration of the antigen bound to the beads was dropped gradually from 200 nM to 1 nM from the first to the fifth round. After phage binding, the beads were subjected to five washing rounds and the phage particles bound to the target protein were eluted by 5 min incubation in 100 μL of 1 U/mL thrombin (1.3 U/μL, Novagen). Then, the phage eluate was used for E. coli infection and phage amplification as described above. After 10³ and higher specificity enrichment of phage was achieved, the infected cells (without the helper phage) were directly plated on ampicillin agar for the overnight growth at 37° C. and sets of 96 colonies were picked to produce phage clones for single-point phage ELISA assays (27). The promising clones demonstrating high specific and low non-specific binding were sequenced and reformatted into a pSFV4 vector as described above for Fab expression and purification.

3. Fab^{LRT}-GA1 Purification and Crystallization—

Recombinant Fab^{LRT}11M (42) and protein GA1 containing 10×His tag (SEQ ID NO: 270) and the TEV-cleavage site at the N-terminus were produced as described above. Prior to the complex formation, 10×His tag (SEQ ID NO: 270) on GA1 was removed using TEV protease. To obtain the Fab^{LRT}-GA1 complex, Fab^{LRT}11M was incubated with GA1 at 1:1 molar ratio on ice for 3 hours and the complex was purified by size-exclusion chromatography on a Superdex 200 Increase 10/300 GL (GE Healthcare Life Sciences) column equilibrated with 20 mM HEPES, 150 mM sodium chloride, pH 7.5. The purity of the complex was confirmed by SDS-PAGE.

Initial crystallization trials of the complex were set up at room temperature using the hanging-drop vapor-diffusion method utilizing the Mosquito Crystal robot (TTP Labtech). Fab^{LRT}-GA1 complex at 17 mg/ml was crystallized by mixing 100 nL of protein complex solution with 100 nL of a Protein Complex Suite (QIAGEN) screen solution. The most promising crystals of Fab^{LRT}-GA1 were observed in 0.1 M magnesium chloride, 0.1 M sodium acetate pH 5.0, and 15% (w/v) PEG 4000 at 19° C. To improve crystal quality the initial crystallization condition was optimized. Hanging-drop crystallization trials were set up at room temperature by mixing 1 μL of complex solution with 1 μL of reservoir solution. Good quality crystals were obtained by the seeding technique (43) in 0.1 M magnesium chloride, 0.1 M sodium acetate pH 5.0, and 20% (w/v) PEG 4000 at 19° C. The resulting crystals of Fab^{LRT}-GA1 were soaked in mother liquor containing 20% glycerol and flash-frozen in liquid nitrogen for data collection.

4. Data Collection, Structure Determination and Refinement—

X-ray diffraction experiments were carried out at 1000 K at beam line 23-ID-D at the General Medical Sciences and Cancer Institute Structural Biology Facility (GM/CA), Argonne National Laboratory (Argonne, IL). Data were indexed and integrated with XDS (44) and scaled using AIMLESS (45) integrated into the CCP4 program suite (46). Initially, the data set was processed in P6222 space group. However, molecular replacement failed to find a structure solution. Evaluation of images showed that there were split reflections at high resolution, which suggested twinning. To explore the possibility of twinning, data were reprocessed in P61, P321 and P312 point group symmetries. The best structure solution with R_{factor}=33.5% and R_{free}=37.8% was obtained in the P3221 space group by molecular replacement method using BALBES (47). A starting PDB model for the Fab^{LRT}-PGA1 complex structure was generated by BALBES based on proteins sequence similarity. The analysis by phenix.xtriage (48) indicated crystal twinning with one twin operator (-h,-k, l) and estimated a twin fraction of 0.49. The structure was refined in PHENIX (48) using obtained twin law to R_{work}=19.2% and R_{free}=25% compared to R_{work}=29.4% and R_{free}=37.1% with no twin refinement. Manual structure corrections were performed in Coot (49, 50). Atom contacts and structure validation were determined in MolProbity (51, 52). The data collection and refinement statistics are summarized in Table S4. The surface accessible solvent area between Fab^{LRT} and PGA1 was calculated in AREAIMOL (53). Structure alignment was performed using CCP4 support program LSKAB (46). Structural figures were created with CCP4 mg (54). Coordinates and structure factors have been deposited in the Protein Data Bank under entry 6U8C (55).

5. Surface Plasmon Resonance (SPR) Analysis—

For SPR analysis, all the protein components were dialyzed into EB buffer. The experiments were performed on a BIAcore-3000 (Biacore AB, Uppsala, Sweden). The target were immobilized via a 10× or 6×His tag (SEQ ID NOS 270 and 269, respectively) to a Ni-NTA chip (GE Healthcare), while Fab variants in 2-fold dilutions were run as analytes in EB buffer (10 mM HEPES, pH 7.4, 150 mM NaCl, 50 μM EDTA, 0.005% Tween20) at 30 μL/min flow rate, 20° C. Senogram traces were corrected through double referencing and fit using Scrubber (BioLogic Software, Campbell, Australia) to a 1:1 binding model.

6. PCA β-Lactamase Assay—

PCA reaction components: BLF-GA1 fusions and the antigen to be detected (viral proteins or Asf1) were combined on ice in 100 μL PBS containing 2 uM fluorogenic BL substrate, Fluorocillin Green 495/525 (Life Technologies), in a well of black FluoroNunc 96-well microplate (Nunc) and the fluorescent signal was monitored at room temperature using Safire2 Tecan Plate Reader (483 nm excitation, 525 nm emission).

7. T-Cell Redirection Cell-Culture Assays—

Human breast cancer cell line SKBR3 (ATCC), overexpressing Her2 gene product on the cell surface was cultured according to ATCC protocols. CD3+ PMBC cells were isolated from patient blood (56, 57) and stored frozen in liquid nitrogen.

The day before the experiment, SKBR3 cells were seeded into a 96-well plate (20K SKBR3 cells in 100 μL per well), while defrosted PBMC were placed into a suspension culture (2 mln cell/ml). After 16 to 24 hours incubation, PBMC cells were washed, transferred to the medium-aspired SKBR3 wells at 10:1 Effector cell to Target cell ratio and then the bi-specific components were added at 50 nM, unless otherwise stated, in the final volume of 100 μL/well. After 24 h of co-culturing, the medium in each plate was analyzed using commercially available kits: for LDH presence (CytoTox96, Promega #G1781, positive control-complete cancer-cell lysis), and cytokine release (INFg, Cisbio #62HIFNGPEG) and (IL2, Cisbio #62HIL02PEG)—the values were normalized using protocols and standards provided in the kits.

D. Supplemental:

1. Fab-Binding Properties

These new Protein Gs are different from pGA1 only in aa 38-43. Kappa, “Lambda” and LRT Fab scaffolds are different from 4D5 scaffold only in the shown Lc region.

Some preliminary SPR results:

a. Binding Affinity, nM

| | K DEQLKSGT | “Λ” SEELQANK | 4D5 DSQLKSGT | LRT D--LRTGT |
|---|---|---|---|---|
| Protein G | (SEQ ID NO: 18) | (SEQ ID NO: 19) | (SEQ ID NO: 271) | (SEQ ID NO: 17) |
| A1 ³⁸YAYVHE⁴³ | nb | nb | 100 | 0.5 |
| (SEQ ID NO: 9) | | | | |
| F ³⁸YAFGNG⁴ | 3 | 50 | 60 | 6 |
| (SEQ ID NO: 10) | | | | |
| D ³⁸IDMVSS⁴³ | 15 | 50 | 80 | nb |
| (SEQ ID NO: 11_{—} | | | | |

b. Kinetic Constants

| | K DEQLKSGT | | “Λ” SEELQANK | | 4D5 DSQLKSGT | | LRT D--LRTGT | |
|---|---|---|---|---|---|---|---|---|
| | (SEQ ID NO: 18) | | (SEQ ID NO: 19) | | (SEQ ID NO: 271) | | (SEQ ID NO: 17) | |
| Protein G | kon | koff | kon | koff | kon | koff | kon | koff |
| A1 ³⁸YAYVHE⁴³ | nb | | nb | | 2e5 | 0.02 | 1e6 | 3e−4 |
| (SEQ ID NO: 9) | | | | | | | | |
| F ³⁸YAFGNG⁴ | 5e5 | 1.5e−3 | 2e5 | 0.01 | 2e5 | 0.02 | 6e5 | 0.003 |
| (SEQ ID NO: 10) | | | | | | | | |

| | | | | | | | |
|---|---|---|---|---|---|---|---|
| D ³⁸IDMVSS⁴³ | 3.2e5 | 1e−2 | 9.3e5 | 7e−3 | 5.9e5 | 5.4e−2 | nb |
| (SEQ ID NO: 11) | | | | | | | |

GF Stability in Sanitization Solution—Needed if Used for Fab Purification

(PAB:120 mM phosphoric acid, 167 mM acetic acid, 2.2% benzyl alcohol, Millipore)

PAB Paper: Rogers M, Hiraoka-Sutow M, Mak P, Mann F, Lebreton B. Development of a rapid sanitization solution for silica-based protein A affinity adsorbents. J Chromatogr A. 2009 May 22; 1216(21):4589-96. doi: 10.1016/j.chroma.2009.03.065. Epub 2009 Mar. 28. PubMed PMID: 19371876.

The inventors were evaluating Protein G stability in PAB solution using the Dot-Blot Assay: Blocking buffer: PBS; 5% BSA; Washing buffer: PBS; 0.02% Tween-20; 0.1% BSA; 0.2 μL protein G dots (1 μM) were placed on a nitrocellulose membrane and allowed to dry. The membrane was blocked in Blocking Buffer for 10 min at RT with shaking and rinsed briefly with water twice. Next, the membranes were submerged in PBS or PAB solution and slowly agitated for 1 h or 20 hours at RT. After 2 washes with water and washing buffer, 20 minutes incubation with 1 ml 100 nM Biotinylated Kappa (“Lambda”) Fabs followed, the membranes were washed three times and incubated with 100 nM Alexa488 Streptavidin for 5 min. After 3 quick washes membranes were dried and imaged at Alexa 488 setting.

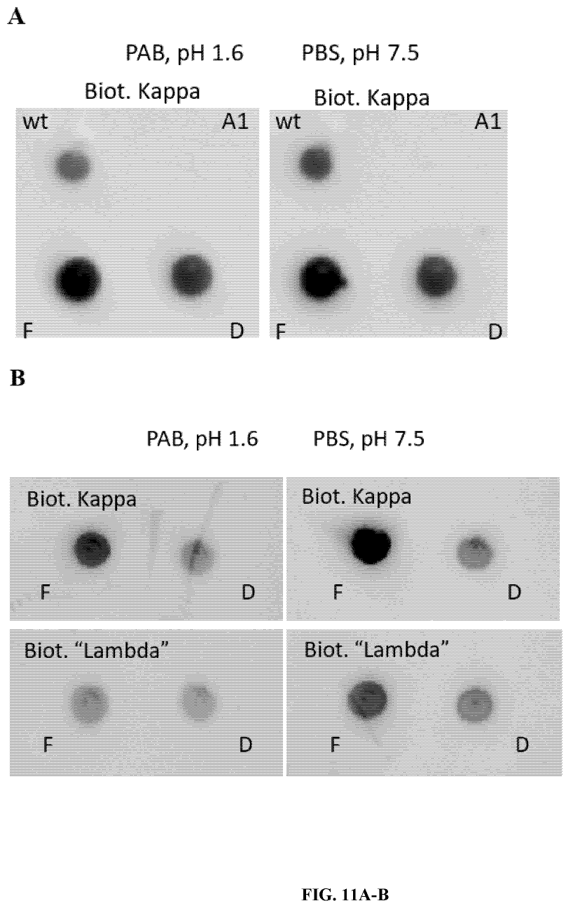

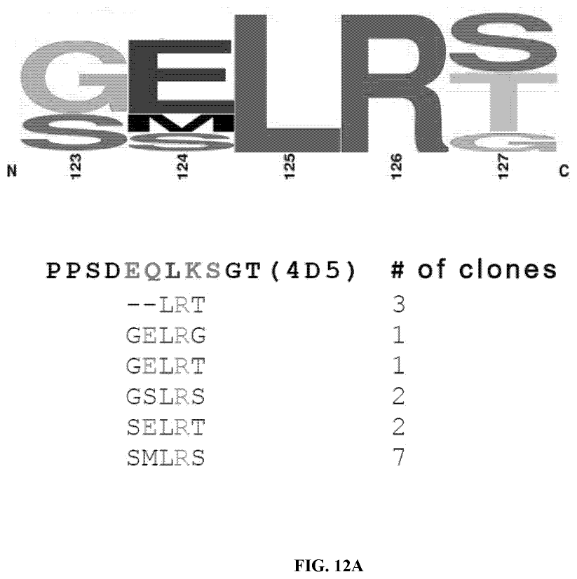

Results: Proteins tested: pGwt, pGA1, pGF and pGD. FIG. 11A shows that 1 h RT PAB—no visible change in pGF or pGD Kappa-Fab binding capacity. FIG. 11B shows 20 h RT PAB—no more than 50% loss in Fab binding capacity.

### Example 2: A New High Affinity Fragment Antibody Binder (FAB)-Chimeric-Antigen Receptor (CAR) Split System for Cancer Immunotherapy

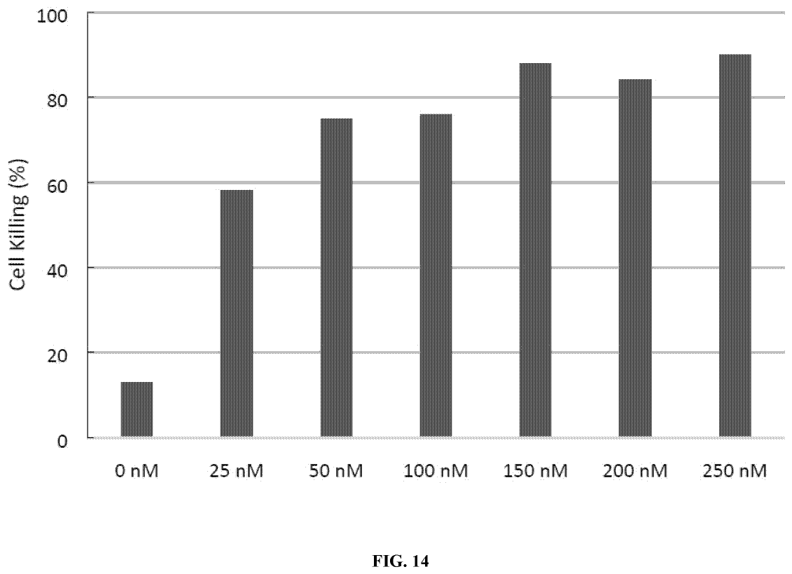

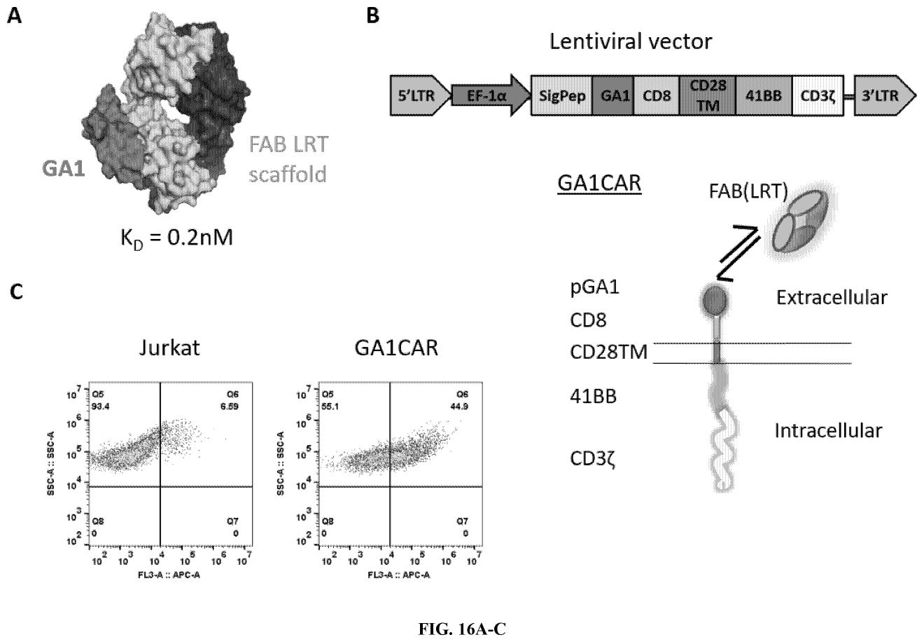

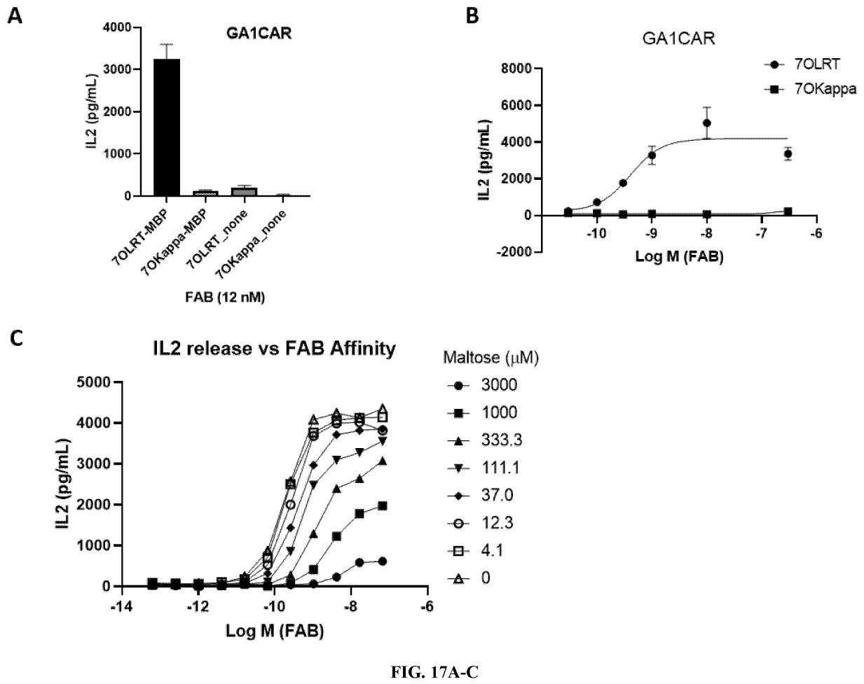

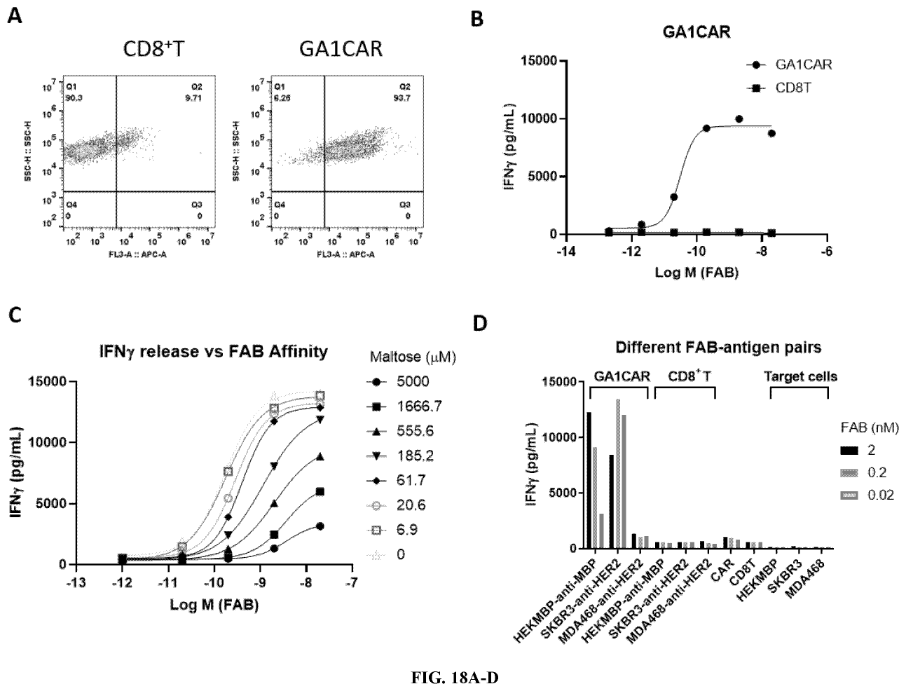

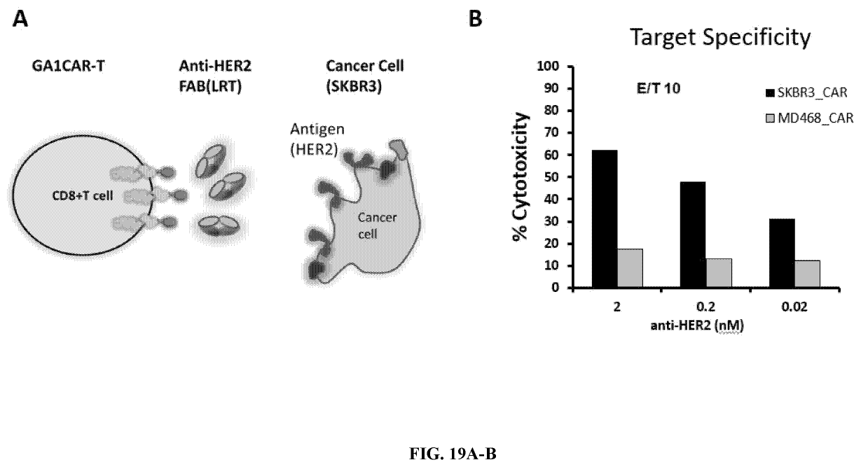

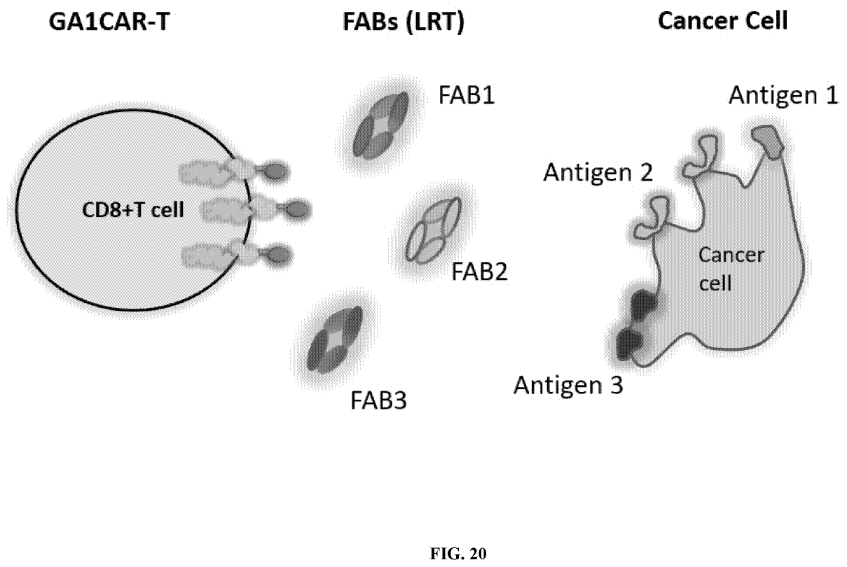

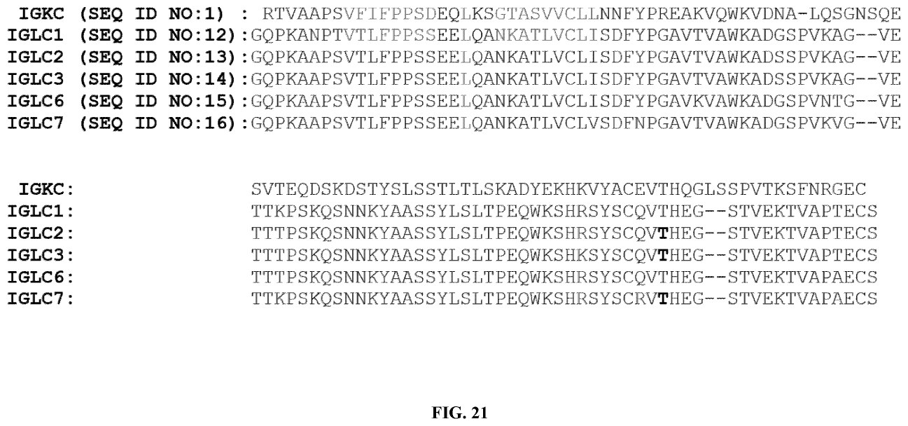

The use of chimeric antigen receptor T (CAR-T) cells for the treatment of hematological cancer has shown extraordinary success, however the use CAR-T for solid tumor therapy faces many challenges. Some of these challenges are on target-off tumor toxicity, target heterogeneity, and precise dose delivery. Here the inventors present a new fragment antibody binder (FAB)-chimeric-antigen receptor (CAR) pair for cancer immunotherapy applications, which overcome some of these challenges. The system is based on an engineered protein G variant (GA1) and a FAB scaffold (LRT) that present exquisite specificity. As a model system we used a FAB binding to maltose binding protein MBP whose affinity is titratable by maltose concentration. This allowed us to provide new insights into this novel pair system in immortalized T cells and peripheral blood mononuclear cells. We then applied our GA1CAR-T to different FAB-antigen pairs to show the versatility of the system. These experiments are further detailed in FIGS. 12-20.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims. All the references, publications, and sequences associated with the recited GenBank Accession numbers are specifically incorporated by reference for all purposes.

### REFERENCES

The following references and those cited throughout the disclosure (including patent documents and non-patent literature), to the extent that they provide exemplary procedural or other details supplementary to those set forth herein, are each specifically incorporated herein by reference each in its entirety.
1. Kohler G, Milstein C. 1975. Continuous cultures of fused cells secreting antibody of predefined specificity. Nature. 256(5517):495-497.
2. Bradbury A R, Sidhu S, Dubel S, McCafferty J. 2011. Beyond natural antibodies: The power of in vitro display technologies. Nat Biotechnol. 29(3):245-254.
3. Groff K, Brown J, Clippinger A J. 2015. Modern affinity reagents: Recombinant antibodies and aptamers. Biotechnol Adv. 33(8):1787-1798.
4. Boder E T, Wittrup K D. 1997. Yeast surface display for screening combinatorial polypeptide libraries. Nat Biotechnol. 15(6):553-557.
5. Bradbury A, Pluckthun A. 2015. Reproducibility: Standardize antibodies used in research. Nature. 518(7537):27-29.
6. Hornsby M, Paduch M, Miersch S, Saaf A, Matsuguchi T, Lee B, Wypisniak K, Doak A, King D, Usatyuk S et al. 2015. A high through-put platform for recombinant antibodies to folded proteins. Mol Cell Proteomics. 14(10):2833-2847.
7. Bass S, Greene R, Wells J A. 1990. Hormone phage: An enrichment method for variant proteins with altered binding properties. Proteins. 8(4):309-314.
8. Paduch M, Koide A, Uysal S, Rizk S S, Koide S, Kossiakoff A A. 2013. Generating conformation-specific synthetic antibodies to trap proteins in selected functional states. Methods. 60(1):3-14.
9. Ye J D, Tereshko V, Frederiksen J K, Koide A, Fellouse F A, Sidhu S S, Koide S, Kossiakoff A A, Piccirilli J A. 2008. Synthetic antibodies for specific recognition and crystallization of structured ma. Proc Natl Acad Sci USA. 105(1):82-87.
10. Paduch M, Kossiakoff A A. 2017. Generating conformation and complex-specific synthetic antibodies. Methods Mol Biol. 1575:93-119.
11. Gao J, Sidhu S S, Wells J A. 2009. Two-state selection of conformation-specific antibodies. Proc Natl Acad Sci USA. 106(9):3071-3076.
12. Miersch S, Li Z, Hanna R, McLaughlin M E, Hornsby M, Matsuguchi T, Paduch M, Saaf A, Wells J, Koide S et al. 2015. Scalable high throughput selection from phage-displayed synthetic antibody libraries. J Vis Exp. (95):51492.
13. Fellouse F A, Esaki K, Birtalan S, Raptis D, Cancasci V J, Koide A, Jhurani P, Vasser M, Wiesmann C, Kossiakoff A A et al. 2007. High-throughput generation of synthetic antibodies from highly functional minimalist phage-displayed libraries. J Mol Biol. 373(4):924-940.
14. Rizk S S, Kouadio J L, Szymborska A, Duguid E M, Mukherjee S, Zheng J, Clevenger C V, Kossiakoff A A. 2015. Engineering synthetic antibody binders for allosteric inhibition of prolactin receptor signaling. Cell Commun Signal. 13:1.
15. Rizk S S, Paduch M, Heithaus J H, Duguid E M, Sandstrom A, Kossiakoff A A. 2011. Allosteric control of ligand-binding affinity using engineered conformation-specific effector proteins. Nat Struct Mol Biol. 18(4):437-442.
16. Marcon E, Jain H, Bhattacharya A, Guo H, Phanse S, Pu S, Byram G, Collins B C, Dowdell E, Fenner M et al. 2015. Assessment of a method to characterize antibody selectivity and specificity for use in immunoprecipitation. Nat Methods. 12(8):725-731.
17. Zhang Z, Liang W G, Bailey L J, Tan Y Z, Wei H, Wang A, Farcasanu M, Woods V A, McCord L A, Lee D et al. 2018. Ensemble cryoem elucidates the mechanism of insulin capture and degradation by human insulin degrading enzyme. Elife. 7.
18. Kang Y, Kuybeda O, de Waal P W, Mukherjee S, Van Eps N, Dutka P, Zhou X E, Bartesaghi A, Erramilli S, Morizumi T et al. 2018. Cryo-em structure of human rhodopsin bound to an inhibitory g protein. Nature. 558(7711):553-558.
19. Dominik P K, Borowska M T, Dalmas O, Kim S S, Perozo E, Keenan R J, Kossiakoff A A. 2016. Conformational chaperones for structural studies of membrane proteins using antibody phage display with nanodiscs. Structure. 24(2):300-309.
20. Bailey L J, Sheehy K M, Dominik P K, Liang W G, Rui H, Clark M, Jaskolowski M, Kim Y, Deneka D, Tang W J et al. 2018. Locking the elbow: Improved antibody fab fragments as chaperones for structure determination. J Mol Biol. 430(3):337-347.
21. Bukowska M A, Grutter M G. 2013. New concepts and aids to facilitate crystallization. Curr Opin Struct Biol. 23(3):409-416.
22. Rizk S S, Luchniak A, Uysal S, Brawley C M, Rock R S, Kossiakoff A A. 2009. An engineered substance p variant for receptor-mediated delivery of synthetic antibodies into tumor cells. Proc Natl Acad Sci USA. 106(27):11011-11015.
23. Bailey L J, Sheehy K M, Hoey R J, Schaefer Z P, Ura M, Kossiakoff A A. 2014. Applications for an engineered protein-g variant with a ph controllable affinity to antibody fragments. J Immunol Methods. 415:24-30.
24. Derrick J P, Wigley D B. 1992. Crystal structure of a streptococcal protein g domain bound to an fab fragment. Nature. 359(6397):752-754.
25. Radwanska M J, Jaskolowski M, Davydova E, Derewenda U, Miyake T, Engel D A, Kossiakoff A A, Derewenda Z S. 2018. The structure of the c-terminal domain of the nucleoprotein from the bundibugyo strain of the ebola virus in complex with a pan-specific synthetic fab. Acta Crystallogr D Struct Biol. 74(Pt 7):681-689.
26. Galarneau A, Primeau M, Trudeau L E, Michnick S W. 2002. Beta-lactamase protein fragment complementation assays as in vivo and in vitro sensors of protein protein interactions. Nat Biotechnol. 20(6):619-622.
27. Schaefer Z P, Bailey L J, Kossiakoff A A. 2016. A polar ring endows improved specificity to an antibody fragment. Protein Sci. 25(7):1290-1298.
28. Yu J S, Liao H X, Gerdon A E, Huffman B, Scearce R M, McAdams M, Alam S M, Popernack P M, Sullivan N J, Wright D et al. 2006. Detection of ebola virus envelope using monoclonal and polyclonal antibodies in elisa, surface plasmon resonance and a quartz crystal microbalance immunosensor. J Virol Methods. 137(2):219-228.
29. Chames P, Baty D. 2009. Bispecific antibodies for cancer therapy: The light at the end of the tunnel? MAbs. 1(6):539-547.
30. Sadelain M, Brentjens R, Riviere I. 2013. The basic principles of chimeric antigen receptor design. Cancer Discov. 3(4):388-398.
31. Brinkmann U, Kontermann R E. 2017. The making of bispecific antibodies. MAbs. 9(2):182-212.
32. Chen L, Flies D B. 2013. Molecular mechanisms of t cell co-stimulation and co-inhibition. Nat Rev Immunol. 13(4):227-242.
33. Huehls A M, Coupet T A, Sentman C L. 2015. Bispecific t-cell engagers for cancer immunotherapy. Immunol Cell Biol. 93(3):290-296.
34. Mack M, Riethmuller G, Kufer P. 1995. A small bispecific antibody construct expressed as a functional single-chain molecule with high tumor cell cytotoxicity. Proc Natl Acad Sci USA. 92(15):7021-7025.
35. Bluemel C, Hausmann S, Fluhr P, Sriskandarajah M, Stallcup W B, Baeuerle P A, Kufer P. 2010. Epitope distance to the target cell membrane and antigen size determine the potency of t cell-mediated lysis by bite antibodies specific for a large melanoma surface antigen. Cancer Immunol Immunother. 59(8):1197-1209.
36. Jarantow S W, Bushey B S, Pardinas J R, Boakye K, Lacy E R, Sanders R, Sepulveda M A, Moores S L, Chiu M L. 2015. Impact of cell-surface antigen expression on target engagement and function of an epidermal growth factor receptor x c-met bispecific antibody. J Biol Chem. 290(41):24689-24704.
37. Gebel H M, Lebeck L K, Jensik S C, Webster K, Bray R A. 1989. T cells from patients successfully treated with okt3 do not react with the t-cell receptor antibody. Hum Immunol. 26(2):123-129.
38. Landegren U, Andersson J, Wigzell H. 1984. Mechanism of t lymphocyte activation by okt3 antibodies. A general model for t cell induction. Eur J Immunol. 14(4):325-328.
39. Huang J, Koide A, Makabe K, Koide S. 2008. Design of protein function leaps by directed domain interface evolution. Proc Natl Acad Sci USA. 105(18):6578-6583.
40. Kunkel T A. 1985. Rapid and efficient site-specific mutagenesis without phenotypic selection. Proc Natl Acad Sci USA. 82(2):488-492.
41. Fuh G, Sidhu S S. 2000. Efficient phage display of polypeptides fused to the carboxy-terminus of the m13 gene-3 minor coat protein. FEBS Lett. 480(2-3):231-234.
42. Mukherjee S, Griffin D H, Horn J R, Rizk S S, Nocula-Lugowska M, Malmqvist M, Kim S S, Kossiakoff A A. 2018. Engineered synthetic antibodies as probes to quantify the energetic contributions of ligand binding to conformational changes in proteins. J Biol Chem. 293(8):2815-2828.
43. Luft J R, DeTitta G T. 1999. A method to produce microseed stock for use in the crystallization of biological macromolecules. Acta Crystallogr D Biol Crystallogr. 55(Pt 5):988-993.
44. Kabsch W. 2010. Integration, scaling, space-group assignment and post-refinement. Acta Crystallogr D Biol Crystallogr. 66(Pt 2):133-144.
45. Evans P R, Murshudov G N. 2013. How good are my data and what is the resolution? Acta Crystallogr D Biol Crystallogr. 69(Pt 7):1204-1214.
46. Winn M D, Ballard C C, Cowtan K D, Dodson E J, Emsley P, Evans P R, Keegan R M, Krissinel E B, Leslie A G, McCoy A et al. 2011. Overview of the ccp4 suite and current developments. Acta Crystallogr D Biol Crystallogr. 67(Pt 4):235-242.
47. Long F, Vagin A A, Young P, Murshudov G N. 2008. Balbes: A molecular-replacement pipeline. Acta Crystallogr D Biol Crystallogr. 64(Pt 1):125-132.
48. Afonine P V, Grosse-Kunstleve R W, Echols N, Headd J J, Moriarty N W, Mustyakimov M, Terwilliger T C, Urzhumtsev A, Zwart P H, Adams P D. 2012. Towards automated crystallographic structure refinement with phenix. Refine. Acta Crystallogr D Biol Crystallogr. 68(Pt 4):352-367.
49. Emsley P, Cowtan K. 2004. Coot: Model-building tools for molecular graphics. Acta Crystallogr D Biol Crystallogr. 60(Pt 12 Pt 1):2126-2132.
50. Emsley P, Lohkamp B, Scott W G, Cowtan K. 2010. Features and development of coot. Acta Crystallogr D Biol Crystallogr. 66(Pt 4):486-501.
51. Chen V B, Arendall W B, 3rd, Headd J J, Keedy D A, Immormino R M, Kapral G J, Murray L W, Richardson J S, Richardson D C. 2010. Molprobity: All-atom structure validation for macromolecular crystallography. Acta Crystallogr D Biol Crystallogr. 66(Pt 1):12-21.
52. Davis I W, Leaver-Fay A, Chen V B, Block J N, Kapral G J, Wang X, Murray L W, Arendall W B, 3rd, Snoeyink J, Richardson J S et al. 2007. Molprobity: All-atom contacts and structure validation for proteins and nucleic acids. Nucleic Acids Res. 35(Web Server issue):W375-383.
53. Lee B, Richards F M. 1971. The interpretation of protein structures: Estimation of static accessibility. J Mol Biol. 55(3):379-400.
54. McNicholas S, Potterton E, Wilson K S, Noble M E. 2011. Presenting your structures: The ccp4 mg molecular-graphics software. Acta Crystallogr D Biol Crystallogr. 67(Pt 4):386-394.
55. Berman H M, Westbrook J, Feng Z, Gilliland G, Bhat T N, Weissig H, Shindyalov I N, Bourne P E. 2000. The protein data bank. Nucleic Acids Res. 28(1):235-242.
56. Ferrante A, Thong Y H. 1980. Optimal conditions for simultaneous purification of mononuclear and polymorphonuclear leucocytes from human blood by the hypaque-ficoll method. J Immunol Methods. 36(2):109-117.
57. Vissers M C, Jester S A, Fantone J C. 1988. Rapid purification of human peripheral blood monocytes by centrifugation through ficoll-hypaque and sepracell-mn. J Immunol Methods. 110(2):203-207.

1. A polypeptide comprising a constant region of an antibody light chain, wherein the constant region comprises a substitution/deletion of amino acids corresponding to positions 16-20 of SEQ ID NO:1 of the constant region with the amino acids LRT.

2. A polypeptide comprising a constant region of an antibody light chain, wherein the constant region comprises a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

3. The polypeptide of claim 1 or 2, wherein the constant region comprises the amino acid sequence of DLRTGT (SEQ ID NO:17) in substitution for the amino acids corresponding to positions 15-22 of SEQ ID NO: 1.

4. The polypeptide of any one of claims 1-3, wherein the polypeptide comprises at least 70% sequence identity to SEQ ID NO:2.

5. The polypeptide of any one of claims 1-4, wherein the antibody light chain comprises a kappa antibody light chain.

6. The polypeptide of claim 3, wherein the constant region comprises the amino acid sequence of DLRTGT (SEQ ID NO:17) in substitution for the amino acids at positions corresponding to 16-23 of SEQ ID NOS:12-16 of a lambda antibody light chain.

7. The polypeptide of any one of claims 1-6, wherein the polypeptide comprises an antibody light chain comprising a variable region and a constant region.

8. The polypeptide of any one of claims 1-7, wherein the polypeptide further comprises an antibody heavy chain, or a fragment thereof.

9. The polypeptide of claim 8, wherein the polypeptide further comprises a fragment of a heavy chain and wherein the fragment comprises a heavy chain variable region.

10. The polypeptide of claim 8 or 9, wherein the polypeptide further comprises a fragment of a heavy chain and wherein the fragment comprises a heavy chain region of a fragment antigen binding (Fab).

11. The polypeptide of any one of claims 8-10, wherein the antibody heavy chain or fragment thereof is carboxy-proximal to the light chain constant region.

12. The polypeptide of any one of claims 8-10, wherein the antibody heavy chain or fragment thereof is amino-proximal to the light chain constant region.

13. The polypeptide of any one of claims 8-12, wherein the polypeptide comprises an antigen binding fragment or a further antigen binding fragment.

14. The polypeptide of claim 13, wherein the antigen binding fragment comprises one or more of a single chain variable fragment (scFv), a single domain antibody, a single chain antibody, and the heavy and/or light chain of a Fab.

15. The polypeptide of any one of claims 7-14, wherein the heavy and or light chain of the polypeptide and/or the antigen binding fragment specifically binds to a tumor antigen, an inflammatory or anti-inflammatory cytokine, a T cell surface receptor, a microbial antigen, a bacterial antigen, or a cell-specific surface protein.

16. The polypeptide of claim 15, wherein the heavy and light chains of the polypeptide specifically bind to a T cell surface receptor, and wherein the T cell surface receptor comprises CD3.

17. The polypeptide of any one of claims 13-16, wherein the antigen binding fragment is carboxy-proximal to the light chain constant region.

18. The polypeptide of any one of claims 13-16, wherein the antigen binding fragment is amino-proximal to the light chain constant region.

19. The polypeptide of any one of claims 1-18, wherein the polypeptide further comprises a Fab binding domain.

20. The polypeptide of claim 19, wherein the Fab binding domain comprises a protein G Fab binding domain.

21. The polypeptide of claim 20, wherein the protein G Fab binding domain comprises a modified protein G Fab binding domain.

22. The polypeptide of claim 21, wherein the modified protein G Fab binding domain comprising a modified isotype recognition region, wherein the isotype recognition region is modified to YAYVHE (SEQ ID NO:9), YAFGNG (SEQ ID NO:10), or IDMVSS (SEQ ID NO:11).

23. The polypeptide of claim 21 or 22, wherein the protein G Fab binding domain comprises one of SEQ ID NO:3-5 or 256.

24. The polypeptide of any one of claims 1-23, wherein the polypeptide further comprises an accessory molecule.

25. The polypeptide of any one of claims 1-24, wherein the polypeptide further comprises one or more linkers.

26. The polypeptide of claim 25, wherein the linker is 100-150 Å.

27. The polypeptide of claim 25, wherein the linker comprises 20-30 amino acid residues.

28. A polypeptide comprising a Fab comprising a heavy chain region and a light chain region, wherein the light chain region comprises a constant region comprising a substitution/deletion of amino acids corresponding to positions 16-20 of SEQ ID NO:1 of the constant region with the amino acids LRT wherein the Fab is conjugated to a protein G Fab binding domain comprising a modified isotype recognition region, wherein the isotype recognition region is modified to YAYVHE (SEQ ID NO:9), YAFGNG (SEQ ID NO:10), or IDMVSS (SEQ ID NO:11).

29. A polypeptide comprising a Fab comprising a heavy chain region and a light chain region, wherein the light chain region comprises a constant region comprising a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T, wherein the Fab is conjugated to a protein G Fab binding domain comprising a modified isotype recognition region, wherein the isotype recognition region is modified to YAYVHE (SEQ ID NO:9), YAFGNG (SEQ ID NO:10), or IDMVSS (SEQ ID NO:11).

30. The polypeptide of claim 28 or 29, wherein the constant region comprises the amino acid sequence of DLRTGT (SEQ ID NO:17) in substitution for the amino acids corresponding to positions 15-22 of SEQ ID NO: 1.

31. The polypeptide of any one of claims 28-30, wherein the polypeptide comprises at least 70% sequence identity to SEQ ID NO:2.

32. The polypeptide of any one of claims 28-30, wherein the antibody light chain comprises a kappa antibody light chain.

33. The polypeptide of claim 30, wherein the constant region comprises the amino acid sequence of DLRTGT (SEQ ID NO:17) in substitution for the amino acids at positions corresponding to 16-23 of SEQ ID NOS:12-16 of a lambda antibody light chain.

34. A polypeptide comprising a Fab conjugated to a protein G Fab binding domain comprising a modified isotype recognition region, wherein the isotype recognition region is modified to YAYVHE (SEQ ID NO:9), YAFGNG (SEQ ID NO:10), or IDMVSS (SEQ ID NO:11).

35. The polypeptide of claim 34, wherein the Fab and the protein G Fab binding domain have no significant binding affinity.

36. The polypeptide of claim 35, wherein the Fab comprises the amino acid sequence of DEQLKSGT (SEQ ID NO:18) or SEELQANK (SEQ ID NO:19) at amino acid positions corresponding to positions 15-22 or SEQ ID NO:1.

37. The polypeptide of claim 35, wherein the Fab does not comprise the amino acid sequence of DLRTGT (SEQ ID NO:17) in substitution for the amino acid residues at positions corresponding to positions 15-22 or SEQ ID NO:1.

38. The polypeptide of any one of claims 28-37, wherein the isotype recognition region is modified to YAYVHE (SEQ ID NO:9).

39. The polypeptide of any one of claims 28-38, wherein the Fab specifically binds to a T cell surface receptor or a tumor antigen.

40. The polypeptide of claim 39, wherein the Fab specifically binds to a T cell surface receptor and wherein the T cell surface receptor comprises CD3.

41. The polypeptide of claim 39, wherein the Fab specifically binds to a tumor antigen and wherein the tumor antigen comprises CD19 or CD20.

42. The polypeptide of any one of claims 28-41, wherein the protein G Fab binding domain comprises an amino acid sequence with at least 70% sequence identity to one of SEQ ID NO:3-8 or 256-257.

43. The polypeptide of any one of claims 28-42, wherein the protein G Fab binding domain comprises an amino acid sequence with at least 70% sequence identity to SEQ ID NO:3.

44. The polypeptide of any one of claims 28-43, wherein the heavy and light chain regions of the Fab are conjugated through a linker.

45. The polypeptide of claim 44, wherein the light chain region is amino-proximal to the heavy chain region.

46. The polypeptide of claim 44, wherein the light chain region is carboxy-proximal to the heavy chain region.

47. The polypeptide of any one of claims 28-46, wherein the heavy and light chain regions of the Fab are conjugated to the protein G Fab binding domain through a linker.

48. The polypeptide of any one of claims 44-47, wherein the linker is less than 100 Å.

49. The polypeptide of any one of claims 44-47, wherein the linker comprises 10-20 amino acid residues.

50. The polypeptide of any one of claims 28-49, wherein the protein G Fab binding domain is amino-proximal to the Fab.

51. The polypeptide of any one of claims 28-49, wherein the protein G Fab binding domain is carboxy-proximal to the Fab.

52. The polypeptide of any one of claims 28-44, wherein the heavy and light chain regions of the Fab are linked through binding affinity and wherein the heavy and light chain are not conjugated through a peptide bond.

53. The polypeptide of claim 52, wherein the protein G Fab binding domain is conjugated to the light chain region of the Fab through a linker.

54. The polypeptide of claim 52, wherein the protein G Fab binding domain is conjugated to the heavy chain region of the Fab through a linker.

55. The polypeptide of claim 53 or 54, wherein the protein G Fab binding domain is carboxy-proximal to the heavy or light chain region of the Fab.

56. The polypeptide of claim 53 or 54, wherein the protein G Fab binding domain is amino-proximal to the heavy or light chain region of the Fab.

57. The polypeptide of any one of claims 28-56, wherein the polypeptide comprises a further antigen binding fragment.

58. The polypeptide of claim 57, wherein the antigen binding fragment comprises one or more of a single chain variable fragment (scFv), a single domain antibody, a single chain antibody, and the heavy and/or light chain of a Fab.

59. The polypeptide of claims 57 or 58, wherein the antigen binding fragment specifically binds to a tumor antigen, an inflammatory or anti-inflammatory cytokine, a T cell surface receptor, or a cell-specific surface protein.

60. A polypeptide comprising a Fab comprising a heavy chain region and a light chain region, wherein the light chain region comprises a constant region comprising a substitution/deletion of amino acids corresponding to positions 16-20 of SEQ ID NO:1 of the constant region with the amino acids LRT and wherein the heavy and/or light chain region of the Fab is conjugated through a linker to a polypeptide comprising a peptide spacer, a transmembrane domain, and an endodomain.

61. A polypeptide comprising a Fab comprising a heavy chain region and a light chain region, wherein the light chain region comprises a constant region comprising a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T, and wherein the heavy and/or light chain region of the Fab is conjugated through a linker to a polypeptide comprising a peptide spacer, a transmembrane domain, and an endodomain.

62. The polypeptide of claim 60 or 61, wherein the constant region comprises the amino acid sequence of DLRTGT (SEQ ID NO: 17) in substitution for the amino acids corresponding to positions 15-22 of SEQ ID NO:1.

63. The polypeptide of any one of claims 60-62, wherein the polypeptide comprises at least 70% sequence identity to SEQ ID NO:2.

64. The polypeptide of any one of claims 57-63, wherein the light chain region comprises a kappa light chain.

65. The polypeptide of claim 62, wherein the constant region comprises the amino acid sequence of DLRTGT (SEQ ID NO:17) in substitution for the amino acids at positions corresponding to 16-23 of SEQ ID NOS:12-16 of a lambda antibody light chain.

66. The polypeptide of any one of claims 60-65, wherein the heavy and light chain regions of the Fab are conjugated through a linker.

67. The polypeptide of claim 66, wherein the light chain region is amino-proximal to the heavy chain region.

68. The polypeptide of claim 66, wherein the light chain region is carboxy-proximal to the heavy chain region.

69. The polypeptide of any one of claims 60-68, wherein the polypeptide comprising the peptide spacer, transmembrane domain, and endodomain is amino-proximal to the heavy and/or light chain region of the Fab.

70. The polypeptide of any one of claims 60-68, wherein the polypeptide comprising the peptide spacer, transmembrane domain, and endodomain is carboxy-proximal to the heavy and/or light chain region of the Fab.

71. The polypeptide of any one of claims 60-61, wherein the heavy and light chain regions of the Fab are linked through binding affinity and wherein the heavy and light chain regions are not conjugated through a peptide bond.

72. The polypeptide of claim 71, wherein the polypeptide comprising the peptide spacer, transmembrane domain, and endodomain is conjugated to the light chain region of the Fab through a linker.

73. The polypeptide of claim 71, wherein the polypeptide comprising the peptide spacer, transmembrane domain, and endodomain is conjugated to the heavy chain region of the Fab through a linker.

74. The polypeptide of claim 72 or 73, wherein the linker is 100-150 Å.

75. The polypeptide of claim 72 or 73, wherein the linker comprises 20-30 amino acid residues.

76. The polypeptide of any one of claims 72-75, wherein the polypeptide comprising the peptide spacer, transmembrane domain, and endodomain is carboxy-proximal to the heavy or light chain region of the Fab.

77. The polypeptide of any one of claims 72-75, wherein the polypeptide comprising the peptide spacer, transmembrane domain, and endodomain is amino-proximal to the heavy or light chain region of the Fab.

78. A polypeptide comprising a protein G Fab binding domain comprising a modified isotype recognition region, wherein the isotype recognition region is modified to YAYVHE (SEQ ID NO:9), YAFGNG (SEQ ID NO: 10), or IDMVSS (SEQ ID NO:11), and wherein the polypeptide further comprises a peptide spacer, a transmembrane domain, and an endodomain.

79. The polypeptide of claim 78, wherein the protein G Fab binding domain further comprises a substitution of the amino acid corresponding to position 19 of SEQ ID NO:23

80. The polypeptide of claim 79, wherein the substitution of the amino acid corresponding to position 19 of SEQ ID NO:3 is with a glutamic acid.

81. The polypeptide of claim 80, wherein the isotype recognition region is modified to YAYVHE (SEQ ID NO:9).

82. The polypeptide of 78 any one of claims 78-81, wherein the protein G Fab binding domain comprises an amino acid sequence with at least 70% sequence identity to one of SEQ ID NO:3-8 or 256-257.

83. The polypeptide of claim 82, wherein the protein G Fab binding domain comprises an amino acid sequence with at least 70% sequence identity to SEQ ID NO:3.

84. The polypeptide of any one of claims 78-83, wherein the protein G Fab binding domain is amino-proximal to the peptide spacer, transmembrane domain, and/or endodomain.

85. The polypeptide of any one of claims 78-83, wherein the protein G Fab binding domain is carboxy-proximal to the to the peptide spacer, transmembrane domain, and/or endodomain.

86. The polypeptide of any one of claims 60-85, wherein the polypeptide comprises an antigen binding fragment or a further antigen binding fragment.

87. The polypeptide of claim 86, wherein the antigen binding fragment comprises one or more of a single chain variable fragment (scFv), a single domain antibody, a single chain antibody, and the heavy and/or light chain of a Fab.

88. The polypeptide of any one of claims 60-87, wherein the Fab and/or antigen binding fragment binds specifically to a tumor antigen, a tumor matrix protein, a cell-specific peptide, a tumor-associated protein, or a T cell surface receptor.

89. The polypeptide of claim 88, wherein the Fab or antigen binding fragment specifically binds to a tumor antigen and wherein the tumor antigen comprises CD19 or CD20.

90. The polypeptide of any one of claims 60-89, wherein the polypeptide has the structure: X-PS-T-E or wherein X comprises the Fab or protein G binding protein, PS is the peptide spacer, T is the transmembrane domain, and E is the endodomain.

91. The polypeptide of any one of claims 60-90, wherein the polypeptide further comprises a co-stimulatory region.

92. The polypeptide of claim 91, wherein the co-stimulatory region is between the transmembrane domain and endodomain.

93. A Fab comprising a constant region of an antibody light chain, wherein the constant region comprises a substitution/deletion of amino acids corresponding to positions 16-20 of SEQ ID NO:1 of the constant region with the amino acids LRT.

94. A Fab comprising a constant region of an antibody light chain, wherein the constant region comprises a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

95. The Fab of claim 93 or 94, wherein the constant region comprises the amino acid sequence of DLRTGT (SEQ ID NO:17) in substitution for the amino acids corresponding to positions 15-22 of SEQ ID NO: 1.

96. The Fab of any one of claims 93-95, wherein the polypeptide comprises at least 70% sequence identity to SEQ ID NO:2.

97. The Fab of any one of claims 93-96, wherein the light chain region comprises a kappa light chain.

98. The Fab of claim 95, wherein the constant region comprises the amino acid sequence of DLRTGT (SEQ ID NO:17) in substitution for the amino acids at positions corresponding to 16-23 of SEQ ID NOS:12-16 of a lambda antibody light chain.

99. The Fab of any one of claims 93-98, wherein the light chain and heavy chain are conjugated through a peptide bond.

100. The Fab of claim 99, wherein the light chain is carboxy-proximal to the heavy chain.

101. The Fab of claim 99, wherein the light chain is amino-proximal to the heavy chain.

102. The Fab of any one of claims 93-98, wherein the light chain and heavy chain are unconjugated.

103. The Fab of any one of claims 93-102, wherein the light chain or the heavy chain of the Fab is conjugated through a peptide bond to a further antigen binding fragment.

104. The Fab of claim 103, wherein the antigen binding fragment comprises a single chain variable fragment (scFv), a single domain antibody, or a single chain antibody.

105. The Fab of any one of claims 93-104, wherein the Fab and/or the antigen binding fragment specifically binds to a tumor antigen, an inflammatory or anti-inflammatory cytokine, a T cell surface receptor, a microbial antigen, a bacterial antigen, or a cell-specific surface protein.

106. The Fab of any one of claims 103-105, wherein the antigen binding fragment is carboxy-proximal to the light chain or heavy chain of the Fab.

107. The Fab of any one of claim 103-105, wherein the antigen binding fragment is amino-proximal to the light chain or heavy chain of the Fab.

108. The Fab of any one of claims 93-107, wherein the wherein the light chain or the heavy chain of the Fab is conjugated through a peptide bond to a Fab binding domain.

109. The Fab of claim 108, wherein the Fab binding domain comprises a protein G Fab binding domain.

110. The Fab of claim 109, wherein the protein G Fab binding domain comprises a modified protein G Fab binding domain.

111. The polypeptide of claim 110, wherein the protein G Fab binding domain further comprises a substitution of the amino acid corresponding to position 19 of SEQ ID NO:23

112. The polypeptide of claim 111, wherein the substitution of the amino acid corresponding to position 19 of SEQ ID NO:3 is with a glutamic acid.

113. The Fab of any one of claims 110-112, wherein the protein G Fab binding domain comprises one of SEQ ID NO:3-5 or 256.

114. The Fab of any one of claims 93-113, wherein the heavy or light chain of the Fab is conjugated to an accessory molecule.

115. The Fab of any one of claims 93-114, wherein the Fab is conjugated to a further polypeptide through one or more linkers.

116. The Fab of claim 115, wherein the linker is 100-150 Å. 139.3 The method of claim.

117. The Fab of claim 115, wherein the linker comprises 20-30 amino acid residues.

118. A polypeptide comprising a modified protein G Fab binding domain comprising an isotype recognition region having the following amino acid sequence: YAFGNG (SEQ ID NO:10).

119. The polypeptide of claim 118, wherein the polypeptide comprises an amino acid sequence with at least 70% sequence identity to SEQ ID NO:4 or 7.

120. A polypeptide comprising a modified protein G Fab binding domain comprising an isotype recognition region having the following amino acid sequence: IDMVSS (SEQ ID NO:11).

121. The polypeptide of claim 120, wherein the polypeptide comprises an amino acid sequence with at least 70% sequence identity to SEQ ID NO:5 or 8.

122. A polypeptide comprising a modified protein G Fab binding domain comprising an isotype recognition region having the following amino acid sequence: YAYVHE (SEQ ID NO:9) and wherein the protein G Fab binding domain further comprises a substitution of the amino acid corresponding to position 19 of SEQ ID NO:23.

123. The polypeptide of claim 122, wherein the polypeptide comprises an amino acid sequence with at least 70% sequence identity to SEQ ID NO:256 or 257.

124. The polypeptide of any one of claims 118-121, wherein the protein G Fab binding domain further comprises a substitution of the amino acid corresponding to position 19 of SEQ ID NO:23

125. The polypeptide of any one of claims 122-124, wherein the substitution of the amino acid corresponding to position 19 of SEQ ID NO:3 is with a glutamic acid.

126. The polypeptide of any one of claims 118-125, wherein the polypeptide further comprises one or more F_{c} regions.

127. The polypeptide of any one of claim 118-126, wherein the polypeptide further comprises a targeting moiety.

128. The polypeptide of any one of claim 118-126, wherein the polypeptide comprises at least two protein G Fab binding domains or at least two modified protein G Fab binding domains.

129. The polypeptide of claim 128, wherein at least one of the modified protein G Fab binding domains comprises an isotype recognition region having the following amino acid sequence: YAYVHE (SEQ ID NO:9).

130. The polypeptide of claim 129, wherein at least one of the modified protein G Fab binding domains comprises an amino acid sequence with at least 70% sequence identity to SEQ ID NO:3 or 8.

131. The polypeptide of any one of claims 118-130, wherein the polypeptide further comprises one or more antigen binding fragments.

132. The polypeptide of claim 131, wherein the one or more antigen binding fragments comprise one or more of a single chain variable fragment (scFv), a single domain antibody, a single chain antibody, and the heavy and/or light chain of a Fab.

133. The polypeptide of claim 131 or 132, wherein the antigen binding fragment(s) specifically bind to a tumor antigen, a T cell surface receptor, a microbial antigen, a bacterial antigen, or a cell-specific surface protein.

134. The polypeptide of any one of claims 131-133, wherein the antigen binding fragment is carboxy-proximal to the Fab binding domain.

135. The polypeptide of any one of claims 131-133, wherein the antigen binding fragment is amino-proximal to the Fab binding domain.

136. The polypeptide of any one of claims 128-135, wherein the Fab binding domain(s) and/or antigen binding fragment(s) are conjugated though one or more linkers.

137. The polypeptide of claim 136, wherein the linker is 100-150 k.

138. The polypeptide of claim 136, wherein the linker comprises 20-30 amino acid residues.

139. A polypeptide comprising a Fab comprising a heavy chain region and a kappa light chain region, wherein the light chain region comprises a constant region comprising a substitution/deletion of amino acids corresponding to positions 16-20 of SEQ ID NO:1 of the constant region with the amino acids LRT wherein the Fab is conjugated to a protein G Fab binding domain comprising a modified isotype recognition region and wherein the isotype recognition region is modified to YAYVHE (SEQ ID NO:9); and further wherein the Fab specifically binds to a T cell surface receptor.

140. A polypeptide comprising a Fab conjugated to a protein G Fab binding domain comprising a modified isotype recognition region, wherein the isotype recognition region is modified to YAYVHE (SEQ ID NO:9) and wherein the Fab specifically binds to a T cell surface receptor.

141. A polypeptide comprising a Fab comprising a heavy chain region and a light chain region, wherein the light chain region comprises a kappa constant region comprising a substitution/deletion of amino acids corresponding to positions 16-20 of SEQ ID NO:1 of the constant region with the amino acids LRT and wherein the heavy and/or light chain region of the Fab is conjugated through a linker to a polypeptide comprising a peptide spacer, a transmembrane domain, and an endodomain.

142. A polypeptide comprising a protein G Fab binding domain comprising a modified isotype recognition region, wherein the isotype recognition region is modified to YAYVHE (SEQ ID NO:9), and wherein the polypeptide further comprises a peptide spacer, a transmembrane domain, and an endodomain.

143. A nucleic acid encoding for the polypeptide of any one of claims 1-92 or 118-142 or the heavy or light chain of the Fab of any one of claims 93-117.

144. A host cell comprising the nucleic acid of claim 143.

145. A therapeutic cell comprising the polypeptide of any one of claims 60-92.

146. The therapeutic cell of claim 145, wherein the cell is an immune cell or an induced pluripotent stem cell.

147. The therapeutic cell of claim 146, wherein the cell is a T cell, a regulatory T cell, a natural killer T cell, or an invariant natural killer T cell.

148. The therapeutic cell of claim 147, wherein the cell is a CD4+ or CD8+ T cell.

149. The therapeutic cell of any one of claims 145-148, wherein the cell is ex vivo.

150. A pharmaceutical composition comprising the polypeptide of any one of claims 1-92 or 118-142, the heavy or light chain of the Fab of any one of claims 93-117, or the therapeutic cell of any one of claims 145-149.

151. A method comprising expressing the nucleic of claim 143 in a host cell and isolating the polypeptides expressed from the nucleic acid.

152. A method for treating a subject comprising administering the polypeptide of any one of claims 1-92 or 118-142, the heavy or light chain of the Fab of any one of claims 93-117, or the therapeutic cell of any one of claims 145-149.

153. The method of claim 152, wherein the method is for treating cancer, an autoimmune condition, reducing an inflammatory response, a viral infection, or a microbial infection.

154. The method of claim 152, wherein the method further comprises administering a polypeptide comprising a constant region of an antibody light chain, wherein the constant region comprises a substitution/deletion of amino acids corresponding to positions 16-20 of SEQ ID NO:1 of the constant region with the amino acids LRT.

155. The method of claim 152, wherein the method further comprises administering a polypeptide comprising a constant region of an antibody light chain, wherein the constant region comprises a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

156. A method for treating cancer in a subject comprising administering:
a) a polypeptide comprising a first Fab conjugated to a protein G Fab binding domain comprising a modified isotype recognition region, wherein the isotype recognition region is modified to YAYVHE (SEQ ID NO:9) and wherein the Fab specifically binds to a T cell surface receptor; and
b) a polypeptide comprising a second Fab that specifically binds to a tumor antigen; and wherein the second Fab comprises a kappa constant region of an antibody light chain, wherein the constant region comprises:
i) a substitution/deletion of amino acids corresponding to positions 16-20 of SEQ ID NO:1 of the constant region with the amino acids LRT; or
ii) a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

157. A method for treating cancer in a subject comprising administering a T cell comprising:
a) a polypeptide comprising a Fab comprising a heavy chain region and a light chain region, wherein the light chain region comprises a kappa constant region comprising a substitution/deletion of amino acids corresponding to positions 16-20 of SEQ ID NO:1 of the constant region with the amino acids LRT and wherein the heavy and/or light chain region of the Fab is conjugated through a linker to a polypeptide comprising a peptide spacer, a transmembrane domain, and an endodomain; and wherein the Fab specifically binds to a tumor antigen; or
b) a nucleic acid encoding a polypeptide comprising a Fab comprising a heavy chain region and a light chain region, wherein the light chain region comprises a kappa constant region comprising a substitution/deletion of amino acids corresponding to positions 16-20 of SEQ ID NO:1 of the constant region with the amino acids LRT and wherein the heavy and/or light chain region of the Fab is conjugated through a linker to a polypeptide comprising a peptide spacer, a transmembrane domain, and an endodomain; and wherein the Fab specifically binds to a tumor antigen.

158. A method for treating cancer in a subject comprising administering:
a) a T cell comprising:
i) a polypeptide comprising a protein G Fab binding domain comprising a modified isotype recognition region, wherein the isotype recognition region is modified to YAYVHE (SEQ ID NO:9), and wherein the polypeptide further comprises a peptide spacer, a transmembrane domain, and an endodomain; or
ii) a nucleic acid encoding a polypeptide comprising a protein G Fab binding domain comprising a modified isotype recognition region, wherein the isotype recognition region is modified to YAYVHE (SEQ ID NO:9), and wherein the polypeptide further comprises a peptide spacer, a transmembrane domain, and an endodomain; and
b) a polypeptide comprising a Fab that specifically binds to a tumor antigen; and wherein the Fab comprises a kappa constant region of an antibody light chain, wherein the constant region comprises:
i) a substitution/deletion of amino acids corresponding to positions 16-20 of SEQ ID NO:1 of the constant region with the amino acids LRT; or
ii) a deletion of amino acids corresponding to positions 16 and 17 of SEQ ID NO:1 and a substitution of amino acids corresponding to positions 19 and 20 of SEQ ID NO:1, wherein the amino acid at position corresponding to 19 of SEQ ID NO:1 is with an R and the amino acid at position corresponding to 20 of SEQ ID NO:1 is with a T.

159. A method for detecting an antigen in a sample comprising
a) incubating the sample with:
i) a first polypeptide comprising at least one protein G Fab binding domain operatively linked to a first component of a detection pair;
ii) a second polypeptide comprising at least one protein G Fab-binding domain operatively linked to a second component of a detection pair;
iii) a first Fab optionally linked or bound to the first modified protein G Fab-binding domain that specifically binds to a first epitope on the antigen; and
iv) a second Fab optionally linked or bound to the second modified protein G Fab-binding domain that specifically binds to a first epitope on the antigen; and
b) detecting the detection pair.

160. The method of claim 159, wherein the detection pair comprises an enzyme and detecting the detection pair comprises detecting enzymatic activity.

161. The method of claim 160, wherein the detection pair comprise a TEM-1 β-lactamase (BL).

162. The method of claim 161, wherein the first component of the detection pair comprises the BLF1 fragment of the TEM-1 BL.

163. The method of claim 161 or 162, wherein the second component of the detection pair comprises the BLF2 fragment of the TEM-1 BL.

164. The method of claim 159, wherein the first and second component of the detection pair comprise a complimentary donor and acceptor fluorophore.

165. The method of any one of claims 159-164, wherein the first Fab comprises a constant region of an antibody light chain, wherein the constant region comprises a substitution/deletion of amino acids corresponding to positions 16-20 of SEQ ID NO:1 of the constant region with the amino acids LRT.

166. The method of any one of claims 159-165, wherein the first protein G binding domain comprises an isotype recognition region having the following amino acid sequence: YAYVHE (SEQ ID NO:9).

167. The method of any one of claims 159-166, wherein the second Fab comprises a human or mouse kappa or lambda light chain.

168. The method of any one of claims 159-167, wherein the second protein G binding domain comprises an isotype recognition region having one of the following amino acid sequences: YAFGNG (SEQ ID NO:10) or IDMVSS (SEQ ID NO:11).

169. The method of any one of claim 159-168, wherein the first protein G Fab-binding domain has a higher affinity for the first Fab compared to the second Fab, and the second protein G Fab-binding domain has a higher affinity for the second Fab compared to the first Fab.

170. The method of any one of claims 159-169, wherein the protein G Fab-binding domain comprises an amino acid sequence with at least 70% homology to one of SEQ ID NOS:3-5 or 256.

171. The method of claim 170, wherein the protein G Fab-binding domain comprises an amino acid sequence of SEQ ID NO:3.

172. The method of any one of claims 159-171, wherein the first polypeptide is linked to the first detection pair through a linker and/or wherein the second polypeptide is linked to the second detection pair through a linker.

173. The method of claim 172, wherein the linker is 100-150 Å.

174. The method of claim 172, wherein the linker comprises 20-30 amino acid residues.

175. The method of any one of claims 159-174, wherein the first or second polypeptide further comprises one or more of F_{c} region(s), targeting moieties, accessory molecules, and combinations thereof.

176. A kit comprising
a) a first polypeptide comprising a protein G Fab-binding domain operatively linked to a first component of a detection pair; and
b) a second polypeptide comprising a protein G Fab-binding domain operatively linked to a second component of a detection pair.

177. The kit of claim 176, further comprising instructions for use.

178. The kit of claim 176 or 177 wherein the detection pair comprises an enzyme.

179. The kit of claim 178 further comprising a substrate.


SEQUENCE LISTING (275 sequences)

SEQ ID NO: 1 - protein, 107 aa, Artificial Sequence [sequence omitted]

SEQ ID NO: 2 - protein, 31 aa, Artificial Sequence
  Description of Artificial Sequence Synthetic polypeptide
  MOD_RES (1)..(1): K, R, E, or I
  MOD_RES (3)..(3): L, F, or A
  MOD_RES (4)..(4): K, S, W, R, or T
  MOD_RES (5)..(5): G or Y
  MOD_RES (6)..(6): E, Y, A, or H
  MOD_RES (7)..(7): T or R
  MOD_RES (8)..(8): T, S, A, or G
  MOD_RES (10)..(10): E, K, T, or Q
  MOD_RES (11)..(30): Any amino acid
  MISC_FEATURE (11)..(30): This region may encompass 5-20 residues
  MOD_RES (31)..(31): Q or R
  See specification as filed for detailed description of substitutions and preferred embodiments
XTXXXXXXTXXXXXXXXXXXXXXXXXXXXXX

SEQ ID NO: 3 - protein, 29 aa, Artificial Sequence
  Description of Artificial Sequence Synthetic peptide
RTLSGYTTTTAVDAATAEKVFKQYAYVHE

SEQ ID NO: 4 - protein, 29 aa, Artificial Sequence
  Description of Artificial Sequence Synthetic peptide
RTLSGYTTTTAVDAATAEKVFKQYAFGNG

SEQ ID NO: 5 - protein, 29 aa, Artificial Sequence
  Description of Artificial Sequence Synthetic peptide
RTLSGYTTTTAVDAATAEKVFKQIDMVSS

SEQ ID NO: 6 - protein, 65 aa, Artificial Sequence
  Description of Artificial Sequence Synthetic polypeptide
TPAVTTYKLVINGRTLSGYTTTTAVDAATAEKVFKQYAYVHEVDGEWTYDDATKTFTVTEKPEKL

SEQ ID NO: 7 - protein, 65 aa, Artificial Sequence
  Description of Artificial Sequence Synthetic polypeptide
TPAVTTYKLVINGRTLSGYTTTTAVDAATAEKVFKQYAFGNGVDGEWTYDDATKTFTVTEKPEKL

SEQ ID NO: 8 - protein, 65 aa, Artificial Sequence
  Description of Artificial Sequence Synthetic polypeptide
TPAVTTYKLVINGRTLSGYTTTTAVDAATAEKVFKQIDMVSSVDGEWTYDDATKTFTVTEKPEKL

SEQ ID NO: 9 - protein, 6 aa, Artificial Sequence
  Description of Artificial Sequence Synthetic peptide
YAYVHE

SEQ ID NO: 10 - protein, 6 aa, Artificial Sequence
  Description of Artificial Sequence Synthetic peptide
YAFGNG

SEQ ID NO: 11 - protein, 6 aa, Artificial Sequence
  Description of Artificial Sequence Synthetic peptide
IDMVSS

SEQ ID NO: 12 - protein, 106 aa, Artificial Sequence
  Description of Artificial Sequence Synthetic polypeptide
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 13 - protein, 106 aa, Artificial Sequence
  Description of Artificial Sequence Synthetic polypeptide
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 14 - protein, 106 aa, Artificial Sequence
  Description of Artificial Sequence Synthetic polypeptide
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 15 - protein, 106 aa, Artificial Sequence
  Description of Artificial Sequence Synthetic polypeptide
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 16 - protein, 106 aa, Artificial Sequence
  Description of Artificial Sequence Synthetic polypeptide
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 17 - protein, 6 aa, Artificial Sequence
  Description of Artificial Sequence Synthetic peptide
DLRTGT

SEQ ID NO: 18 - protein, 8 aa, Artificial Sequence
  Description of Artificial Sequence Synthetic peptide
DEQLKSGT

SEQ ID NO: 19 - protein, 8 aa, Artificial Sequence
  Description of Artificial Sequence Synthetic peptide
SEELQANK

SEQ ID NO: 20 - protein, 480 aa, Streptococcus sp.
EFNKYGVSDYYKNLINNAKTVEGVKDLQAQVVESAKKARISEATDGLSDFLKSQTPAEDTVKSIELAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTEVPGDAPTEPEKPEASIPLVPLTPATPIAKDDAKKDDTKKEDAKKPEAKKEDAKKAETLPTTGEGSNPFFTAAALAVMAGAGALAVASKRKED

SEQ ID NO: 21 - protein, 593 aa, Streptococcus sp.
MEKEKKVKYFLRKSAFGLASVSAAFLVGSTVFAVDSPIEDTPIIRNGGELTNLLGNSETTLALRNEESATADLTAAAVADTVAAAAAENAGAAAWEAAAAADALAKAKADALKEFNKYGVSDYYKNLINNAKTVEGVKDLQAQVVESAKKARISEATDGLSDFLKSQTPAEDTVKSIELAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTEVPGDAPTEPEKPEASIPLVPLTPATPIAKDDAKKDDTKKEDAKKPEAKKEDAKKAETLPTTGEGSNPFFTAAALAVMAGAGALAVASKRKED

SEQ ID NO: 22 - protein, 185 aa, Streptococcus sp.
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 23 - protein, 29 aa, Streptococcus sp.
KTLKGETTTKAVDAATAEKVFKQYANDNG

SEQ ID NO: 24 - protein, 29 aa, Streptococcus sp.
KTLKGETTTEAVDAATAEKVFKQYANDNG

SEQ ID NO: 25 - protein, 29 aa, Streptococcus sp.
KTLKGETTTKAVDAETAEKAFKQYANDNG

SEQ ID NO: 26 - protein, 5 aa, Artificial Sequence
  Description of Artificial Sequence Synthetic peptide
GSLRS

SEQ ID NO: 27 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGRTLSGYTTTTAVDAATAEKVFKQYAYVHEVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 28 - protein, 8 aa, Artificial Sequence
  Description of Artificial Sequence Synthetic peptide
PEELRTNK

SEQ ID NO: 29 - protein, 6 aa, Artificial Sequence
  Description of Artificial Sequence Synthetic peptide
YSRPHV

SEQ ID NO: 30 - protein, 6 aa, Artificial Sequence
  Description of Artificial Sequence Synthetic peptide
YAYGAV

SEQ ID NO: 31 - protein, 29 aa, Artificial Sequence
  Description of Artificial Sequence Synthetic peptide
KTFWGETTTKAVDAATAEKVFKQYAFDND

SEQ ID NO: 32 - protein, 29 aa, Artificial Sequence
  Description of Artificial Sequence Synthetic peptide
ETLRYETSTKAVDAATAEKVFKQIAHDQG

SEQ ID NO: 33 - protein, 29 aa, Artificial Sequence
  Description of Artificial Sequence Synthetic peptide
KTLKGETTTKAVDAATAEKVFKQYAYVHD

SEQ ID NO: 34 - protein, 29 aa, Artificial Sequence
  Description of Artificial Sequence Synthetic peptide
ETLRYETSTKAVDAATAEKVFKRIAHDQG

SEQ ID NO: 35 - protein, 29 aa, Artificial Sequence
  Description of Artificial Sequence Synthetic peptide
KTASGARATKAVDAATAEKVFKQYAKEYP

SEQ ID NO: 36 - protein, 29 aa, Artificial Sequence
  Description of Artificial Sequence Synthetic peptide
ETLTGETGTQAVDAATAEKVFKQYAWVND

SEQ ID NO: 37 - protein, 29 aa, Artificial Sequence
  Description of Artificial Sequence Synthetic peptide
ITLKGHTTTKAVDAATAEKVFKQYAWVND

SEQ ID NO: 38 - protein, 4 aa, Artificial Sequence
  Description of Artificial Sequence Synthetic peptide
GGGS

SEQ ID NO: 39 - protein, 12 aa, Artificial Sequence
  Description of Artificial Sequence Synthetic peptide
GGGSGGGSGGGS

SEQ ID NO: 40 - protein, 4 aa, Artificial Sequence
  Description of Artificial Sequence Synthetic peptide
LAAA

SEQ ID NO: 41 - protein, 19 aa, Artificial Sequence
  Description of Artificial Sequence Synthetic peptide
LGGGGSGGGGSGGGGSAAA

SEQ ID NO: 42 - protein, 25 aa, Artificial Sequence
  Description of Artificial Sequence Synthetic peptide
LSGGGGSGGGGSGGGGSGGGGSAAA

SEQ ID NO: 43 - protein, 15 aa, Artificial Sequence
  Description of Artificial Sequence Synthetic peptide
LAEAAAKEAAAKAAA

SEQ ID NO: 44 - protein, 20 aa, Artificial Sequence
  Description of Artificial Sequence Synthetic peptide
LAEAAAKEAAAKEAAAKAAA

SEQ ID NO: 45 - protein, 25 aa, Artificial Sequence
  Description of Artificial Sequence Synthetic peptide
LAEAAAKEAAAKEAAAKEAAAKAAA

SEQ ID NO: 46 - protein, 30 aa, Artificial Sequence
  Description of Artificial Sequence Synthetic polypeptide
LAEAAAKEAAAKEAAAKEAAAKEAAAKAAA

SEQ ID NO: 47 - protein, 11 aa, Streptococcus sp.
KLVINGRTLSG

SEQ ID NO: 48 - protein, 6 aa, Streptococcus sp.
YANDNG

SEQ ID NO: 49 - protein, 6 aa, Artificial Sequence
  Description of Artificial Sequence Synthetic peptide
YAYVHE

SEQ ID NO: 50 - protein, 6 aa, Artificial Sequence
  Description of Artificial Sequence Synthetic peptide
YSRPHV

SEQ ID NO: 51 - protein, 6 aa, Artificial Sequence
  Description of Artificial Sequence Synthetic peptide
YAVGAV

SEQ ID NO: 52 - protein, 6 aa, Artificial Sequence
  Description of Artificial Sequence Synthetic peptide
YAAPHV

SEQ ID NO: 53 - protein, 6 aa, Artificial Sequence
  Description of Artificial Sequence Synthetic peptide
YSHPHV

SEQ ID NO: 54 - protein, 6 aa, Artificial Sequence
  Description of Artificial Sequence Synthetic peptide
CTVWPV

SEQ ID NO: 55 - protein, 6 aa, Artificial Sequence
  Description of Artificial Sequence Synthetic peptide
YAFAHV

SEQ ID NO: 56 - protein, 5 aa, Artificial Sequence
  Description of Artificial Sequence Synthetic peptide
SMLRS

SEQ ID NO: 57 - protein, 10 aa, Streptococcus sp.
LVINGRTLSG

SEQ ID NO: 58 - protein, 10 aa, Artificial Sequence
  Description of Artificial Sequence Synthetic peptide
LVIRGLTLSL

SEQ ID NO: 59 - protein, 10 aa, Artificial Sequence
  Description of Artificial Sequence Synthetic peptide
LVIRGLTLSF

SEQ ID NO: 60 - protein, 10 aa, Artificial Sequence
  Description of Artificial Sequence Synthetic peptide
LVIGGLRLWF

SEQ ID NO: 61 - protein, 10 aa, Artificial Sequence
  Description of Artificial Sequence Synthetic peptide
LVIRGVTLLF

SEQ ID NO: 62 - protein, 10 aa, Artificial Sequence
  Description of Artificial Sequence Synthetic peptide
LVIRGITLGF

SEQ ID NO: 63 - protein, 10 aa, Artificial Sequence
  Description of Artificial Sequence Synthetic peptide
LVIMGSTLSL

SEQ ID NO: 64 - protein, 10 aa, Artificial Sequence
  Description of Artificial Sequence Synthetic peptide
LVIIGRTLSL

SEQ ID NO: 65 - protein, 10 aa, Artificial Sequence
  Description of Artificial Sequence Synthetic peptide
LVISGITLSF

SEQ ID NO: 66 - protein, 10 aa, Artificial Sequence
  Description of Artificial Sequence Synthetic peptide
LVIGGRTLSF

SEQ ID NO: 67 - protein, 10 aa, Artificial Sequence
  Description of Artificial Sequence Synthetic peptide
LVIGGRTLSF

SEQ ID NO: 68 - protein, 10 aa, Artificial Sequence
  Description of Artificial Sequence Synthetic peptide
LVISGSTLSL

SEQ ID NO: 69 - protein, 10 aa, Artificial Sequence
  Description of Artificial Sequence Synthetic peptide
LVILGRTLSV

SEQ ID NO: 70 - protein, 10 aa, Artificial Sequence
  Description of Artificial Sequence Synthetic peptide
FVIRGRTLSF

SEQ ID NO: 71 - protein, 10 aa, Artificial Sequence
  Description of Artificial Sequence Synthetic peptide
LVISGRTLSL

SEQ ID NO: 72 - protein, 10 aa, Artificial Sequence
  Description of Artificial Sequence Synthetic peptide
LVIGGRTLRF

SEQ ID NO: 73 - protein, 10 aa, Artificial Sequence
  Description of Artificial Sequence Synthetic peptide
LVIRGVTLGF

SEQ ID NO: 74 - protein, 10 aa, Artificial Sequence
  Description of Artificial Sequence Synthetic peptide
LVIRGRTLSL

SEQ ID NO: 75 - protein, 10 aa, Artificial Sequence
  Description of Artificial Sequence Synthetic peptide
LVIGGRTLRF

SEQ ID NO: 76 - protein, 10 aa, Artificial Sequence
  Description of Artificial Sequence Synthetic peptide
LVIGGRTLSF

SEQ ID NO: 77 - protein, 10 aa, Artificial Sequence
  Description of Artificial Sequence Synthetic peptide
LVISGLTLSF

SEQ ID NO: 78 - protein, 10 aa, Artificial Sequence
  Description of Artificial Sequence Synthetic peptide
LVIGGVTLSF

SEQ ID NO: 79 - protein, 10 aa, Artificial Sequence
  Description of Artificial Sequence Synthetic peptide
LVIRGVTLSL

SEQ ID NO: 80 - protein, 10 aa, Artificial Sequence
  Description of Artificial Sequence Synthetic peptide
LVIGGITLSF

SEQ ID NO: 81 - protein, 10 aa, Artificial Sequence
  Description of Artificial Sequence Synthetic peptide
  MOD_RES (1)..(1): L or F
  MOD_RES (4)..(4): N, R, G, M, I, S, or L
  MOD_RES (6)..(6): R, L, V, I, or S
  MOD_RES (7)..(7): T or R
  MOD_RES (9)..(9): S, W, L, G, or R
  MOD_RES (10)..(10): L, F, or V
  See specification as filed for detailed description of substitutions and preferred embodiments
XVIXGXXLXX

SEQ ID NO: 82 - protein, 12 aa, Artificial Sequence
  Description of Artificial Sequence Synthetic peptide
AVDAATAEKVFK

SEQ ID NO: 83 - protein, 14 aa, Artificial Sequence
  Description of Artificial Sequence Synthetic peptide
LTPAVTTYKLVING

SEQ ID NO: 84 - protein, 23 aa, Artificial Sequence
  Description of Artificial Sequence Synthetic peptide
VDGEWTYDDATKTFTVTEKPEVI

SEQ ID NO: 85 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGRTLSGYTTTTAVDAATAEKVFKQYAFGNGVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 86 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGRTLSGYTTTTAVDAATAEKVFKQIDMVSSVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 87 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTFWGETTTKAVDAATAEKVFKQYAFDNDVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 88 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGETLRYETSTKAVDAATAEKVFKQIAHDQGVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 89 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYAYVHDVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 90 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGETLRYETSTKAVDAATAEKVFKRIAHDQGVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 91 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTASGARATKAVDAATAEKVFKQYAKEYPVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 92 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGETLTGETGTQAVDAATAEKVFKQYAWVNDVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 93 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGITLKGHTTTKAVDAATAEKVFKQYAWVNDVDGEWTYDDATKTFTVTEKPEVIDASELTPAVTTYKLVINGRTLSGETTTKAVDAETAEKAFKQYANDNGVDGVWTYDDATKTFTVTE

SEQ ID NO: 94 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGRTLSGETTTKAVDAETAEKAFKQYAYVHEVDGVWTYDDATKTFTVTE

SEQ ID NO: 95 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGRTLSGETTTKAVDAETAEKAFKQYSRPHVVDGVWTYDDATKTFTVTE

SEQ ID NO: 96 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGRTLSGETTTKAVDAETAEKAFKQYAYGAVVDGVWTYDDATKTFTVTE

SEQ ID NO: 97 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGRTLSGETTTKAVDAETAEKAFKQYAAPHVVDGVWTYDDATKTFTVTE

SEQ ID NO: 98 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGRTLSGETTTKAVDAETAEKAFKQYSHPHVVDGVWTYDDATKTFTVTE

SEQ ID NO: 99 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGRTLSGETTTKAVDAETAEKAFKQCTVWPVVDGVWTYDDATKTFTVTE

SEQ ID NO: 100 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGRTLSGETTTKAVDAETAEKAFKQYAFAHVVDGVWTYDDATKTFTVTE

SEQ ID NO: 101 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIRGLTLSLETTTKAVDAETAEKAFKQYANDNGVDGVWTYDDATKTFTVTE

SEQ ID NO: 102 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIRGLTLSFETTTKAVDAETAEKAFKQYANDNGVDGVWTYDDATKTFTVTE

SEQ ID NO: 103 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIGGLRLWFETTTKAVDAETAEKAFKQYANDNGVDGVWTYDDATKTFTVTE

SEQ ID NO: 104 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIRGVTLLFETTTKAVDAETAEKAFKQYANDNGVDGVWTYDDATKTFTVTE

SEQ ID NO: 105 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIRGITLGFETTTKAVDAETAEKAFKQYANDNGVDGVWTYDDATKTFTVTE

SEQ ID NO: 106 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIMGSTLSLETTTKAVDAETAEKAFKQYANDNGVDGVWTYDDATKTFTVTE

SEQ ID NO: 107 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIIGRTLSLETTTKAVDAETAEKAFKQYANDNGVDGVWTYDDATKTFTVTE

SEQ ID NO: 108 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVISGITLSFETTTKAVDAETAEKAFKQYANDNGVDGVWTYDDATKTFTVTE

SEQ ID NO: 109 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIGGRTLSFETTTKAVDAETAEKAFKQYANDNGVDGVWTYDDATKTFTVTE

SEQ ID NO: 110 - protein, 185 aa, Artificial Sequence; identical to SEQ ID NO: 109
  Description of Artificial Sequence Synthetic polypeptide

SEQ ID NO: 111 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVISGSTLSLETTTKAVDAETAEKAFKQYANDNGVDGVWTYDDATKTFTVTE

SEQ ID NO: 112 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVILGRTLSVETTTKAVDAETAEKAFKQYANDNGVDGVWTYDDATKTFTVTE

SEQ ID NO: 113 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKFVIRGRTLSFETTTKAVDAETAEKAFKQYANDNGVDGVWTYDDATKTFTVTE

SEQ ID NO: 114 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVISGRTLSLETTTKAVDAETAEKAFKQYANDNGVDGVWTYDDATKTFTVTE

SEQ ID NO: 115 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIGGRTLRFETTTKAVDAETAEKAFKQYANDNGVDGVWTYDDATKTFTVTE

SEQ ID NO: 116 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIRGVTLGFETTTKAVDAETAEKAFKQYANDNGVDGVWTYDDATKTFTVTE

SEQ ID NO: 117 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIRGRTLSLETTTKAVDAETAEKAFKQYANDNGVDGVWTYDDATKTFTVTE

SEQ ID NO: 118 - protein, 185 aa, Artificial Sequence; identical to SEQ ID NO: 115
  Description of Artificial Sequence Synthetic polypeptide

SEQ ID NO: 119 - protein, 185 aa, Artificial Sequence; identical to SEQ ID NO: 109
  Description of Artificial Sequence Synthetic polypeptide

SEQ ID NO: 120 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVISGLTLSFETTTKAVDAETAEKAFKQYANDNGVDGVWTYDDATKTFTVTE

SEQ ID NO: 121 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIGGVTLSFETTTKAVDAETAEKAFKQYANDNGVDGVWTYDDATKTFTVTE

SEQ ID NO: 122 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIRGVTLSLETTTKAVDAETAEKAFKQYANDNGVDGVWTYDDATKTFTVTE

SEQ ID NO: 123 - protein, 185 aa, Artificial Sequence
  Description of Artificial Sequence Synthetic polypeptide
MKGETTTEAVDAATAEKVFKQYANDNGVDGEWTYDDATKTFTVTEKPEVIDASELTPAVTTYKLVINGKTLKGETTTKAVDAATAEKVFKQYANDNGVDGEWTYDDATKTFTVTEKPEVIDASELTPAVTTYKLVIGGITLSFETTTKAVDAETAEKAFKQYANDNGVDGVWTYDDATKTFTVTE

SEQ ID NO: 124 - protein, 13 aa, Artificial Sequence
  Description of Artificial Sequence Synthetic peptide
GGSGSAGSGGAGA

SEQ ID NO: 125 - protein, 5 aa, Unknown
  Description of Unknown immunoglobulin hinge region sequence
DKTHT

SEQ ID NO: 126 - protein, 4 aa, Unknown
  Description of Unknown immunoglobulin hinge region sequence
CPPC

SEQ ID NO: 127 - protein, 15 aa, Unknown
  Description of Unknown immunoglobulin hinge region sequence
CPEPKSCDTPPPCPR

SEQ ID NO: 128 - protein, 12 aa, Unknown
  Description of Unknown immunoglobulin hinge region sequence
ELKTPLGDTTHT

SEQ ID NO: 129 - protein, 10 aa, Unknown
  Description of Unknown immunoglobulin hinge region sequence
KSCDKTHTCP

SEQ ID NO: 130 - protein, 7 aa, Unknown
  Description of Unknown immunoglobulin hinge region sequence
KCCVDCP

SEQ ID NO: 131 - protein, 7 aa, Unknown
  Description of Unknown immunoglobulin hinge region sequence
KYGPPCP

SEQ ID NO: 132 - protein, 15 aa, Homo sapiens
EPKSCDKTHTCPPCP

SEQ ID NO: 133 - protein, 12 aa, Homo sapiens
ERKCCVECPPCP

SEQ ID NO: 134 - protein, 17 aa, Homo sapiens
ELKTPLGDTTHTCPRCP

SEQ ID NO: 135 - protein, 12 aa, Unknown
  Description of Unknown immunoglobulin hinge region sequence
SPNMVPHAHHAQ

SEQ ID NO: 136 - protein, 12 aa, Unknown
  Description of Unknown immunoglobulin hinge region sequence
ESKYGPPCPPCP

SEQ ID NO: 137 - protein, 12 aa, Unknown
  Description of Unknown immunoglobulin hinge region sequence
ESKYGPPCPSCP

SEQ ID NO: 138 - protein, 45 aa, Homo sapiens
TTTPAPRPPTPAPTIASQPLSLRPEACRPAAGGAVHTRGLDFACD

SEQ ID NO: 139 - protein, 110 aa, Artificial Sequence
  Description of Artificial Sequence Synthetic polypeptide
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 140 - protein, 107 aa, Artificial Sequence
  Description of Artificial Sequence Synthetic polypeptide
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 141 - protein, 27 aa, Artificial Sequence
  Description of Artificial Sequence Synthetic peptide
FWVLVVVGGVLACYSLLVTVAFIIFWV

SEQ ID NO: 142 - protein, 23 aa, Artificial Sequence
  Description of Artificial Sequence Synthetic peptide
LGLLVAGVLVLLVSLGVAIHLCC

SEQ ID NO: 143 - protein, 25 aa, Artificial Sequence
  Description of Artificial Sequence Synthetic peptide
ALIVLGGVAGLLLFIGLGIFFCVRC

SEQ ID NO: 144 - protein, 23 aa, Artificial Sequence
  Description of Artificial Sequence Synthetic peptide
LCYLLDGILFIYGVILTALFLRV

SEQ ID NO: 145 - protein, 26 aa, Artificial Sequence
  Description of Artificial Sequence Synthetic peptide
WVLVVVGGVLACYSLLVTVAFIIFWV

SEQ ID NO: 146 - protein, 26 aa, Artificial Sequence
  Description of Artificial Sequence Synthetic peptide
VAAILGLGLVLGLLGPLAILLALYLL

SEQ ID NO: 147 - protein, 24 aa, Artificial Sequence
  Description of Artificial Sequence Synthetic peptide
ALPAALAVISFLLGLGLGVACVLA

SEQ ID NO: 148 - protein, 113 aa, Artificial Sequence
  Description of Artificial Sequence Synthetic polypeptide
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 149 - protein, 112 aa, Artificial Sequence
  Description of Artificial Sequence Synthetic polypeptide
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 150 - protein, 102 aa, Artificial Sequence
  Description of Artificial Sequence Synthetic polypeptide
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 151 - protein, 101 aa, Artificial Sequence
  Description of Artificial Sequence Synthetic polypeptide
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 152 - protein, 21 aa, Artificial Sequence
  Description of Artificial Sequence Synthetic peptide
ESPYQELQGQRSDVYSDLNTQ

SEQ ID NO: 153 - protein, 86 aa, Artificial Sequence
  Description of Artificial Sequence Synthetic polypeptide
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 154 - protein, 21 aa, Artificial Sequence
  Description of Artificial Sequence Synthetic peptide
DGVYTGLSTRNQETYETLKHE

SEQ ID NO: 155 - protein, 171 aa, Artificial Sequence
  Description of Artificial Sequence Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 156 - protein, 127 aa, Artificial Sequence
  Description of Artificial Sequence Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 157 - protein, 21 aa, Artificial Sequence
  Description of Artificial Sequence Synthetic peptide
DQVYQPLRDRDDAQYSHLGGN

SEQ ID NO: 158 - protein, 207 aa, Artificial Sequence
  Description of Artificial Sequence Synthetic polypeptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 159 - protein, 21 aa, Artificial Sequence
  Description of Artificial Sequence Synthetic peptide
NPDYEPIRKGQRDLYSGLNQR

SEQ ID NO: 160 - protein, 182 aa, Artificial Sequence
  Description of Artificial Sequence Synthetic polypeptide
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 161 - protein, 21 aa, Artificial Sequence
  Description of Artificial Sequence Synthetic peptide
DQLYQPLKDREDDQYSHLQGN

SEQ ID NO: 162 - protein, 163 aa, Artificial Sequence
  Description of Artificial Sequence Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 164 aa, Artificial Sequence
  Description of Artificial Sequence Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 164 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 112 aa, Artificial Sequence; identical to SEQ ID NO: 164
  Description of Artificial Sequence Synthetic polypeptide

SEQ ID NO: 166 - protein, 21 aa, Artificial Sequence
  Description of Artificial Sequence Synthetic peptide
NQLYNELNLGRREEYDVLDKR

SEQ ID NO: 167 - protein, 22 aa, Artificial Sequence
  Description of Artificial Sequence Synthetic peptide
EGLYNELQKDKMAEAYSEIGMK

SEQ ID NO: 168 - protein, 21 aa, Artificial Sequence
  Description of Artificial Sequence Synthetic peptide
DGLYQGLSTATKDTYDALHMQ

SEQ ID NO: 169 - protein, 42 aa, Unknown
  Description of Unknown transmembrane protein 4-1BB sequence
KRGRKKLLYIFKQPFMRPVQTTQEEDGCSCRFPEEEEGGCEL

SEQ ID NO: 170 - protein, 44 aa, Unknown
  Description of Unknown transmembrane protein CD28 sequence
FWVRSKRSRLLHSDYMNMTPRRPGPTRKHYQPYAPPRDFAAYRS

SEQ ID NO: 171 - protein, 35 aa, Unknown
  Description of Unknown transmembrane protein ICOS sequence
TKKKYSSSVHDPNGEYMFMRAVNTAKKSRLTDVTL

SEQ ID NO: 172 - protein, 37 aa, Unknown
  Description of Unknown transmembrane protein OX-40 sequence
RRDQRLPPDAHKPPGGGSFRTPIQEEQADAHSTLAKI

SEQ ID NO: 173 - protein, 9 aa, Artificial Sequence
  Description of Artificial Sequence Synthetic peptide
YPYDVPDYA

SEQ ID NO: 174 - protein, 8 aa, Artificial Sequence
  Description of Artificial Sequence Synthetic peptide
DYKDDDDK

SEQ ID NO: 175 - protein, 10 aa, Artificial Sequence
  Description of Artificial Sequence Synthetic peptide
EQKLISEEDL

SEQ ID NO: 176 - protein, 5 aa, Artificial Sequence
  Description of Artificial Sequence Synthetic peptide
GSGGS

SEQ ID NO: 177 - protein, 4 aa, Artificial Sequence
  Description of Artificial Sequence Synthetic peptide
GGGS

SEQ ID NO: 178 - protein, 4 aa, Artificial Sequence
  Description of Artificial Sequence Synthetic peptide
GGSG

SEQ ID NO: 179 - protein, 5 aa, Artificial Sequence
  Description of Artificial Sequence Synthetic peptide
GGSGG

SEQ ID NO: 180 - protein, 5 aa, Artificial Sequence
  Description of Artificial Sequence Synthetic peptide
GSGSG

SEQ ID NO: 181 - protein, 5 aa, Artificial Sequence
  Description of Artificial Sequence Synthetic peptide
GSGGG

SEQ ID NO: 182 - protein, 5 aa, Artificial Sequence
  Description of Artificial Sequence Synthetic peptide
GGGSG

SEQ ID NO: 183 - protein, 5 aa, Artificial Sequence
  Description of Artificial Sequence Synthetic peptide
GSSSG

SEQ ID NO: 184 - protein, 213 aa, Artificial Sequence
  Description of Artificial Sequence Synthetic polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQYSYSLVTFGQGTKVEIKRTVAAPSVFIFPPSDLRTGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 235 aa, Artificial Sequence
  Description of Artificial Sequence Synthetic polypeptide
EISEVQLVESGGGLVQPGGSLRLSCAASGFNVYYYYIHWVRQAPGKGLEWVASISPYYGYTSYADSVKGRFTISADTSKNTAYLQMNSLRAEDTAVYYCARWSYDQSMSYKSGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 186 - protein, 8 aa, Artificial Sequence
  Description of Artificial Sequence Synthetic peptide
QSVSSAVA

SEQ ID NO: 187 - protein, 8 aa, Artificial Sequence
  Description of Artificial Sequence Synthetic peptide
YSASSLYS

SEQ ID NO: 188 - protein, 6 aa, Artificial Sequence
  Description of Artificial Sequence Synthetic peptide
YSYSLV

SEQ ID NO: 189 - protein, 6 aa, Artificial Sequence
  Description of Artificial Sequence Synthetic peptide
VYYYYI

SEQ ID NO: 190 - protein, 8 aa, Artificial Sequence
  Description of Artificial Sequence Synthetic peptide
SISPYYGY

SEQ ID NO: 191 - protein, 13 aa, Artificial Sequence
  Description of Artificial Sequence Synthetic peptide
WSYDQSMSYKSGM

SEQ ID NO: 192 - protein, 213 aa, Artificial Sequence
  Description of Artificial Sequence Synthetic polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQSSSSLITFGQGTKVEIKRTVAAPSVFIFPPSDLRTGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 236 aa, Artificial Sequence
  Description of Artificial Sequence Synthetic polypeptide
EISEVQLVESGGGLVQPGGSLRLSCAASGFNISYSSIHWVRQAPGKGLEWVASIYSYSGYTSYADSVKGRFTISADTSKNTAYLQMNSLRAEDTAVYYCARSYWYHVGSWHYTGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 194 - protein, 8 aa, Artificial Sequence
  Description of Artificial Sequence Synthetic peptide
QSVSSAVA

SEQ ID NO: 195 - protein, 8 aa, Artificial Sequence
  Description of Artificial Sequence Synthetic peptide
YSASSLYS

SEQ ID NO: 196 - protein, 6 aa, Artificial Sequence
  Description of Artificial Sequence Synthetic peptide
SSSSLI

SEQ ID NO: 197 - protein, 6 aa, Artificial Sequence
  Description of Artificial Sequence Synthetic peptide
ISYSSI

SEQ ID NO: 198 - protein, 8 aa, Artificial Sequence
  Description of Artificial Sequence Synthetic peptide
SIYSYSGY

SEQ ID NO: 199 - protein, 14 aa, Artificial Sequence
  Description of Artificial Sequence Synthetic peptide
SYWYHVGSWHYTGM

SEQ ID NO: 200 - protein, 213 aa, Artificial Sequence
  Description of Artificial Sequence Synthetic polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQWYDSLITFGQGTKVEIKRTVAAPSVFIFPPSDLRTGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 229 aa, Artificial Sequence
  Description of Artificial Sequence Synthetic polypeptide
EISEVQLVESGGGLVQPGGSLRLSCAASGFNVYYSSIHWVRQAPGKGLEWVAYIYPSSGSTYYADSVKGRFTISADTSKNTAYLQMNSLRAEDTAVYYCARSWSPYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 202 - protein, 8 aa, Artificial Sequence
  Description of Artificial Sequence Synthetic peptide
QSVSSAVA

SEQ ID NO: 203 - protein, 8 aa, Artificial Sequence
  Description of Artificial Sequence Synthetic peptide
YSASSLYS

SEQ ID NO: 204 - protein, 6 aa, Artificial Sequence
  Description of Artificial Sequence Synthetic peptide
WYDSLI

SEQ ID NO: 205 - protein, 6 aa, Artificial Sequence
  Description of Artificial Sequence Synthetic peptide
VYYSSI

SEQ ID NO: 206 - protein, 8 aa, Artificial Sequence
  Description of Artificial Sequence Synthetic peptide
YIYPSSGS

SEQ ID NO: 207 - protein, 7 aa, Artificial Sequence
  Description of Artificial Sequence Synthetic peptide
SWSPYGM

SEQ ID NO: 208 - protein, 213 aa, Artificial Sequence
  Description of Artificial Sequence Synthetic polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQSWSSLFTFGQGTKVEIKRTVAAPSVFIFPPSDLRTGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 235 aa, Artificial Sequence
  Description of Artificial Sequence Synthetic polypeptide
EISEVQLVESGGGLVQPGGSLRLSCAASGFNVYYYYIHWVRQAPGKGLEWVASIYSYSGYTYYADSVKGRFTISADTSKNTAYLQMNSLRAEDTAVYYCAREGVWWEDEFYPGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 210 - protein, 8 aa, Artificial Sequence
  Description of Artificial Sequence Synthetic peptide
QSVSSAVA

SEQ ID NO: 211 - protein, 8 aa, Artificial Sequence
  Description of Artificial Sequence Synthetic peptide
YSASSLYS

SEQ ID NO: 212 - protein, 6 aa, Artificial Sequence
  Description of Artificial Sequence Synthetic peptide
SWSSLF

SEQ ID NO: 213 - protein, 6 aa, Artificial Sequence
  Description of Artificial Sequence Synthetic peptide
VYYYYI

SEQ ID NO: 214 - protein, 8 aa, Artificial Sequence
  Description of Artificial Sequence Synthetic peptide
SIYSYSGY

SEQ ID NO: 215 - protein, 13 aa, Artificial Sequence
  Description of Artificial Sequence Synthetic peptide
EGVWWEDEFYPGL

SEQ ID NO: 216 - protein, 213 aa, Artificial Sequence
  Description of Artificial Sequence Synthetic polypeptide
SDIQMTQSPSSLSASVGDRVTITCRASQSVSSAVAWYQQKPGKAPKLLIYSASSLYSGVPSRFSGSRSGTDFTLTISSLQPEDFATYYCQQSSDDPITFGQGTKVEIKRTVAAPSVFIFPPSDLRTGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 229 aa, Artificial Sequence
  Description of Artificial Sequence Synthetic polypeptide
EISEVQLVESGGGLVQPGGSLRLSCAASGFNISYSSIHWVRQAPGKGLEWVASISSYYGSTYYADSVKGRFTISADTSKNTAYLQMNSLRAEDTAVYYCARSRGQASWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 218 - protein, 8 aa, Artificial Sequence
  Description of Artificial Sequence Synthetic peptide
QSVSSAVA

SEQ ID NO: 219 - protein, 8 aa, Artificial Sequence
  Description of Artificial Sequence Synthetic peptide
YSASSLYS

SEQ ID NO: 220 - protein, 6 aa, Artificial Sequence
  Description of Artificial Sequence Synthetic peptide
SWSSLF

SEQ ID NO: 221 - protein, 6 aa, Artificial Sequence
  Description of Artificial Sequence Synthetic peptide
ISYSSI

SEQ ID NO: 222 - protein, 8 aa, Artificial Sequence
  Description of Artificial Sequence Synthetic peptide
SISSYYGS

SEQ ID NO: 223 - protein, 7 aa, Artificial Sequence
  Description of Artificial Sequence Synthetic peptide
SRGQASW

SEQ ID NO: 224 - protein, 212 aa, Artificial Sequence
  Description of Artificial Sequence Synthetic polypeptide
SDIQMTQSPSSLSASVGDRVTITCSASSSVSYMNWYQQKPGKAPKRWIYDTSKLASGVPSRFSGSGSGTDYTLTISSLQPEDFATYYCQQWSSNPFTFGQGTKVEIKRTVAAPSVFIFPPSDLRTGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 230 aa, Artificial Sequence
  Description of Artificial Sequence Synthetic polypeptide
EISEVQLVESGGGLVQPGGSLRLSCAASGYTFTRYTMHWVRQAPGKGLEWIGYINPSRGYTNYNQKFKDKATISTDKSKNTAYLQMNSLRAEDTAVYYCARYYDDHYSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 226 - protein, 7 aa, Artificial Sequence
  Description of Artificial Sequence Synthetic peptide
SSVSYMN

SEQ ID NO: 227 - protein, 8 aa, Artificial Sequence
  Description of Artificial Sequence Synthetic peptide
YDTSKLAS

SEQ ID NO: 228 - protein, 5 aa, Artificial Sequence
  Description of Artificial Sequence Synthetic peptide
WSSNP

SEQ ID NO: 229 - protein, 9 aa, Artificial Sequence
  Description of Artificial Sequence Synthetic peptide
TFTRYTMHW

SEQ ID NO: 230 - protein, 21 aa, Artificial Sequence
  Description of Artificial Sequence Synthetic peptide
IGYINPSRGYTNYNQKFKDKA

SEQ ID NO: 231 - protein, 8 aa, Artificial Sequence
  Description of Artificial Sequence Synthetic peptide
YYDDHYSL

SEQ ID NO: 232 - protein, 212 aa, Artificial Sequence; identical to SEQ ID NO: 224
  Description of Artificial Sequence Synthetic polypeptide

SEQ ID NO: 233 - protein, 230 aa, Artificial Sequence
  Description of Artificial Sequence Synthetic polypeptide
EISEVQLVESGGGLVQPGGSLRLSCAASGYTFTRYTMHWVRQAPGKGLEWIGYINPSDGYTNYNQKFKDKATISTDKSKNTAYLQMNSLRAEDTAVYYCARYYDDHYSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 234 - protein, 7 aa, Artificial Sequence
  Description of Artificial Sequence Synthetic peptide
SSVSYMN

SEQ ID NO: 235 - protein, 8 aa, Artificial Sequence
  Description of Artificial Sequence Synthetic peptide
YDTSKLAS

SEQ ID NO: 236 - protein, 5 aa, Artificial Sequence
  Description of Artificial Sequence Synthetic peptide
WSSNP

SEQ ID NO: 237 - protein, 9 aa, Artificial Sequence
  Description of Artificial Sequence Synthetic peptide
TFTRYTMHW

SEQ ID NO: 238 - protein, 21 aa, Artificial Sequence
  Description of Artificial Sequence Synthetic peptide
IGYINPSDGYTNYNQKFKDKA

SEQ ID NO: 239 - protein, 8 aa, Artificial Sequence
  Description of Artificial Sequence Synthetic peptide
YYDDHYSL

SEQ ID NO: 240 - protein, 214 aa, Artificial Sequence
  Description of Artificial Sequence Synthetic polypeptide
SDIQMTQSPSSLSASVGDRVTITCSASSSVSYMNWYQQKPGKAPKRWIYDTSKLASGVPSRFSGSGSGTDYTLTISSLQPEDFATYYCQQWSSNPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 308 aa, Artificial Sequence
  Description of Artificial Sequence Synthetic polypeptide
EISEVQLVESGGGLVQPGGSLRLSCAASGYTFTRYTMHWVRQAPGKGLEWIGYINPSRGYTNYNQKFKDKATISTDKSKNTAYLQMNSLRAEDTAVYYCARYYDDHYSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGSAGSGGAGATPAVTTYKLVINGRTLSGYTTTTAVDAATAEKVFKQYAYVHEVDGEWTYDDATKTFTVTEKPEKL

SEQ ID NO: 242 - protein, 7 aa, Artificial Sequence
  Description of Artificial Sequence Synthetic peptide
SSVSYMN

SEQ ID NO: 243 - protein, 7 aa, Artificial Sequence
  Description of Artificial Sequence Synthetic peptide
DTSKLAS

SEQ ID NO: 244 - protein, 6 aa, Artificial Sequence
  Description of Artificial Sequence Synthetic peptide
WSSNPF

SEQ ID NO: 245 - protein, 9 aa, Artificial Sequence
  Description of Artificial Sequence Synthetic peptide
TFTRYTMHW

SEQ ID NO: 246 - protein, 21 aa, Artificial Sequence
  Description of Artificial Sequence Synthetic peptide
IGYINPSRGYTNYNQKFKDKA

SEQ ID NO: 247 - protein, 8 aa, Artificial Sequence
  Description of Artificial Sequence Synthetic peptide
YYDDHYSL

SEQ ID NO: 248 - protein, 213 aa, Artificial Sequence
  Description of Artificial Sequence Synthetic polypeptide
SDIQMTQSPSSLSASVGDRVTITCSASQDIRNYLNWYQQKPGKAPKRWIYYTSRLHSGVPSRFSGSGSGTDYTLTISSLQPEDFATYYCQQGNTLPWTFGQGTKVEIKRTVAAPSVFIFPPSDLRTGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - protein, 233 aa, Artificial Sequence
  Description of Artificial Sequence Synthetic polypeptide
EISEVQLVESGGGLVQPGGSLRLSCAASGFNFTGYTIHWVRQAPGKGLEWMGLINPYKGVSTYNQKFKDKATISTDKSKNTAYLQMNSLRAEDTAVYYCARSGYYGDSDWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 250 - protein, 8 aa, Artificial Sequence
  Description of Artificial Sequence Synthetic peptide
QDIRNYLN

SEQ ID NO: 251 - protein, 9 aa, Artificial Sequence
  Description of Artificial Sequence Synthetic peptide
IYYTSRLHS

SEQ ID NO: 252 - protein, 6 aa, Artificial Sequence
  Description of Artificial Sequence Synthetic peptide
GNTLPW

SEQ ID NO: 253 - protein, 9 aa, Artificial Sequence
  Description of Artificial Sequence Synthetic peptide
NFTGYTIHW

SEQ ID NO: 254 - protein, 21 aa, Artificial Sequence
  Description of Artificial Sequence Synthetic peptide
MGLINPYKGVSTYNQKFKDKA

SEQ ID NO: 255 - protein, 11 aa, Artificial Sequence
  Description of Artificial Sequence Synthetic peptide
SGYYGDSDWYF

SEQ ID NO: 256 - protein, 29 aa, Artificial Sequence
  Description of Artificial Sequence Synthetic peptide
RTLSGYTTTTAVDAATAEEVFKQYAYVHE

SEQ ID NO: 257 - protein, 65 aa, Artificial Sequence
  Description of Artificial Sequence Synthetic polypeptide
TPAVTTYKLVINGRTLSGYTTTTAVDAATAEEVFKQYAYVHEVDGEWTYDDATKTFTVTEKPEKL

SEQ ID NO: 258 - protein, 5 aa, Artificial Sequence
  Description of Artificial Sequence Synthetic peptide
SQLRT

SEQ ID NO: 259 - protein, 5 aa, Artificial Sequence
  Description of Artificial Sequence Synthetic peptide
EQLKS

SEQ ID NO: 260 - protein, 5 aa, Artificial Sequence
  Description of Artificial Sequence Synthetic peptide
EELQA

SEQ ID NO: 261 - protein, 5 aa, Artificial Sequence
  Description of Artificial Sequence Synthetic peptide
EQLTS

SEQ ID NO: 262 - protein, 5 aa, Artificial Sequence
  Description of Artificial Sequence Synthetic peptide
EELET

SEQ ID NO: 263 - DNA, 15 bp, Artificial Sequence
  Description of Artificial Sequence Synthetic oligonucleotide
  modified_base (1)..(2): a, c, t, or g
  modified_base (4)..(5): a, c, t, or g
  modified_base (7)..(8): a, c, t, or g
  modified_base (10)..(11): a, c, t, or g
  modified_base (13)..(14): a, c, t, or g
nnknnknntnnknnk

SEQ ID NO: 264 - protein, 5 aa, Artificial Sequence
  Description of Artificial Sequence Synthetic peptide
GGGGS

SEQ ID NO: 265 - protein, 9 aa, Unknown
  Description of Unknown “LAGLIDADG” Family motif peptide
LAGLIDADG

SEQ ID NO: 266 - protein, 4 aa, Streptococcus sp.
NDNG

SEQ ID NO: 267 - protein, 4 aa, Artificial Sequence
  Description of Artificial Sequence Synthetic peptide
YVHE

SEQ ID NO: 268 - protein, 5 aa, Artificial Sequence
  Description of Artificial Sequence Synthetic peptide
SQLKS

SEQ ID NO: 269 - protein, 6 aa, Artificial Sequence
  Description of Artificial Sequence Synthetic 6xHis tag
HHHHHH

SEQ ID NO: 270 - protein, 10 aa, Artificial Sequence
  Description of Artificial Sequence Synthetic 10xHis tag
HHHHHHHHHH

SEQ ID NO: 271 - protein, 8 aa, Artificial Sequence
  Description of Artificial Sequence Synthetic peptide
DSQLKSGT

SEQ ID NO: 272 - protein, 11 aa, Artificial Sequence
  Description of Artificial Sequence Synthetic peptide
PPSDEQLKSGT

SEQ ID NO: 273 - protein, 5 aa, Artificial Sequence
  Description of Artificial Sequence Synthetic peptide
GELRG

SEQ ID NO: 274 - protein, 5 aa, Artificial Sequence
  Description of Artificial Sequence Synthetic peptide
GELRT

SEQ ID NO: 275 - protein, 5 aa, Artificial Sequence
  Description of Artificial Sequence Synthetic peptide
SELRT